(19) United States
(12) Patent Application Publication
(10) Pub. No.: US 2016/0116490 A1
(43) Pub. Date: Apr. 28, 2016

(54) METHODS AND DEVICES FOR IMMUNODIAGNOSTIC APPLICATIONS

(71) Applicant: ARRYX, INC., Braintree, MA (US)

(72) Inventors: Christopher R. Knutson, Woodridge, IL (US); Sridevi Kurella, Aurora, IL (US); Derek D. Doorneweerd, Lockport, IL (US); Christopher F. Grant, Chicago, IL (US); Timothy R. Kline, State College, PA (US)

(73) Assignee: ARRYX, INC., Braintree, MA (US)

(21) Appl. No.: 14/420,183

(22) PCT Filed: Aug. 7, 2013
(86) PCT No.: PCT/US2013/053960

§ 371 Date: Feb. 6, 2015

Related U.S. Application Data
(60) Provisional application No. 61/681,049, filed on Aug. 8, 2012.

(51) Int. Cl. G01N 33/80

(52) U.S. Cl. CPC G01N 33/80

(57) ABSTRACT

Methods and devices for evaluating a sample, e.g., a plasma sample, from a subject, for detecting a target red blood cell protein or antibody are disclosed. In one embodiment, optimized antibody screening methods and devices significantly reduce the level of non-specific binding to a surface (e.g., a test surface bound with a red blood cell (rbcm) preparation), thus allowing for more efficient detection and reduced test time. In one embodiment, the optimized antibody screening method includes an immunoglobulin G (IgG) binding moiety that binds selectively and specifically to the plasma IgG present relative to the binding to the lysed rbcm preparation. In another embodiment, an optimized antibody screening method is disclosed whereby non-specific binding caused by lysed red blood cell membrane preparations can be reduced by an agent that specifically cleaves a human IgG in the hinge region. In other embodiments, the invention provides methods and devices for target capturing that include a substantially planar surface, optionally having an optimized angle, for capture. Alternative solid phase geometries for capture are disclosed. Optimized methods for cell deposition are also disclosed. Thus, optimized methods, devices, kits, assays for evaluating a sample are disclosed.

### RELATED APPLICATIONS

This application claims the benefit of U.S. Provisional Application No. 61/681,049, filed Aug. 8, 2012, the contents of which are incorporated herein by reference in their entirety.

### BACKGROUND

Approximately 30 human blood group systems are recognized by the International Society of Blood Transfusion (ISBT). These 30 systems are composed of over 600 blood group antigens. The most clinically significant system is the ABO blood group system that, amongst others, includes the A, B, AB, and O blood groups. The second most clinically significant blood-group system is the Rh system. The Rh system currently includes over 50 antigens—the most significant of which is the D antigen. Thus, red blood cells (RBCs) have many antigens on their surfaces, some of which may be associated with “blood group” (Groups A, B, AB, and O), and the most common antigens, known as the A, B, and D antigens, give rise to one's ABO Rh “blood type” commonly listed on the donor cards for people who donate blood (e.g., A Rh Pos, A Rh Neg, B Rh Pos, B Rh Neg, O Rh Pos, O Rh Neg, AB Rh Pos, AB Rh Neg).

Testing for those surface antigens is commonly called either “forward typing” (FT) or “ABO/Rh antigen typing”. This test is performed on every blood donor and every potentially transfused patient, typically at least twice for redundancy.

“Reverse grouping” (RG) or “reverse typing,” refers to determining whether an individual's plasma contains antibodies specific to the A antigen (Anti-A) and/or antibodies specific to the B antigen (Anti-B) in it. If a subject does not have the A antigen on their RBCs, they will have the Anti-A antibody in their plasma. Similarly, if a subject does not have the B antigen on their RBCs, they will have the Anti-B antibody in their blood plasma. In other words, if the subject has the A antigen alone on their RBCs (and thus, Anti-B antibodies are present), their blood type is Group A, and if they have the B antigen alone on their RBCs (and only Anti-A antibodies present), they have Group B blood. Group AB will have both A, B, and AB antigens on the RBCs, but neither Anti-A or Anti-B antibodies present, and Group O will have no A, B, or AB antigens on the RBCs and both Anti-A and Anti-B antibodies present. Effectively, this test provides redundant information to the forward group, and is thus, another check on the result of the forward group. There are many details, but for the most part, there is a simple correspondence.

Hence, to perform a full “ABO/Rh blood type,” forward typing is performed to determine the presence or absence of the A, B, and D antigens on RBCs, and reverse grouping is performed to determine the presence or absence of the Anti-A, Anti-B, or Anti-AB antibodies in the plasma.

The intrinsic presence of antibodies specific to foreign ABO blood groups gives the ABO system its clinical significance. If a transfusion of non-ABO matched red cells occurs, the transfusion recipient will likely experience a transfusion reaction—which may be fatal.

However, as stated above, numerous other antigens corresponding to other blood group systems are present on human red blood cells, generally referred to as “minor antigens.” Amongst these antigens, there are 18 that fall into a second tier of clinical significance after the A, B, and D antigens. These systems are not characterized by the intrinsic presence of antibodies specific to foreign blood groups. Hence, a mismatch between donor and recipient RBC's does not typically result in an immediate transfusion reaction. Instead, a person who is exposed to these foreign antigens through transfused RBCs may, over time, develop antibodies specific to these foreign antigens—i.e., immunization. This can occur through pregnancy (a mother may be exposed to a child's blood and thus antigens on the child's RBCs) or through a blood transfusion. If a subject is transfused with RBCs, they will likely be given RBCs that present one or more these 18 antigens that the subject's own RBCs do not present and the subject may have an immune response. This is typically not medically detrimental to the individual unless the subject is later exposed to additional RBCs (e.g., a second transfusion) that present an antigen to which they have established immunity. If such a second exposure occurs, the immune response will typically be much stronger as antibodies specific to these foreign antigens have been pre-formed and the immune system is primed for rapid production of these antibodies. As a result, the subject's immune system is primed to attack the transfused blood, destroying the donor RBCs and giving rise to various clinical problems. For these reasons, anyone who receives a blood transfusion is screened to determine whether they have antibodies specific to these 18 antigens. This is called “antibody screening” (AbS).

A subject is screened and characterized as either positive or negative for one or more these “unexpected antibodies” that possess specificity for one of the 18 antigens. If the subject has a positive antibody screen, it is then necessary to determine the specificity(ies) of the(se) antibody(ies). This process is called “antibody identification” (AbID). Thus, a patient sample that yields a positive AbS, will undergo an AbID to identify the specificity of the antibodies that are present. Then the hospital or lab must find “antigen negative blood” which does not present the antigens corresponding to the specificity (ies) of the unexpected antibody (ies). Red cell blood units can be tested for the presence or absence of particular minor antigens by performing “extended phenotyping” or “antigen characterization” tests, depending on whether one identifies the presence or absence of many or all 18 antigens (extended phenotyping), or targets one or few specific antigens of interest (minor antigen characterization). Collectively, these are often referred to as “antigen typing.” Generally, units are considered ideal for transfusion if they are compatible with the patient's blood type and are negative for the antigens corresponding to antibodies the patient presents. Minor antigen characterization and extended phenotyping can also performed on patient blood at times, depending on hospital practices. Generally, if a patient has one or more unexpected antibodies, the lab will confirm the patient does not present the corresponding antigen on the red cells. Further, if a patient falls into certain groups, typically groups expected to be multiply transfused, extended phenotypes may be performed. Some hospitals perform an extended phenotype on any patient that presents with antibodies.

Finally, before the transfusion occurs, the hospital performs a “crossmatch”, which, in the U.S., typically requires a “serological crossmatch”. patient plasma and donor RBCs are combined and examined for a reaction (agglutination of the RBCs). If the patient does not have any unexpected antibodies (ie. the AbS was negative), a simple “immediate spin crossmatch” (ISXM) is performed in which the patient plasma and donor RBCs are mixed at room temperature and then inspected for agglutination. If the patient had a positive AbS test the ISXM is performed as well as a “Coombs crossmatch” or “IAT crossmatch” (IATXM) which involves mixing the patients plasma with the donor RBCs, incubating, washing the RBCs, adding anti-human globulin (AHG), allowing for agglutination of the RBCs such as through centrifugation, and then inspection for agglutination. If the crossmatch does not produce a reaction, the blood is released for transfusion.

Common historical methods used for blood typing include combining RBCs of unknown type with antibodies specific to each antigen of interest, or combining RBCs of known type with plasma with unknown antibody content. For example, to perform a forward type, RBCs are combined with three separate solutions—each containing one of anti-A, anti-B, and anti-D IgM class antibodies. If the RBCs present the antigen corresponding to the specificity of the antibody (i.e., RBCs presenting the A antigen combined with anti-A), the antibody will bind to the antigen presented on the surface of the RBCs, produce a ‘bridge’ between the cells, and induce aggregation (hemagglutination). If the RBCs agglutinate when mixed with anti-A, the subject has group A antigens present on the RBCs. If the RBCs agglutinate when mixed with anti-B, the subject has group B RBCs. If the RBCs agglutinate when mixed with both Anti-A and Anti-B antibodies (or anti-AB), then the subject has Group AB RBCs. If the RBCs do not agglutinate with either Anti-A, Anti-B, or anti-AB antibodies, then the subject has Group O RBCs. If the RBCs agglutinate when mixed with anti-D, then the subject has RhD positive RBCs. Finally, if the RBC do not agglutinate when mixed with anti-D, the subject has RhD negative RBCs.

Reverse grouping tests are performed in a similar fashion. However, in this case, plasma or serum of unknown group is combined with separate solutions each containing RBCs of a singular known group (i.e., A, B, or O RBCs). If the plasma or serum contains antibodies specific to the antigens presented on the RBCs, the RBCs will agglutinate. As an example, if plasma is combined with A, B, and O cells and agglutination is observed only in the sample containing A cells, the plasma contains only anti-A and, therefore, the subject has Group B blood. If only the sample containing B cells agglutinates, the plasma only contains anti-B, and, therefore, the subject has Group A blood. If the samples containing A cells and B cells agglutinate, the sample contains both Anti-A and Anti-B, and, therefore, the subject has Group O blood. Finally, if none of the samples containing A cells, B cells, or O cells agglutinate, the sample contains neither Anti-A nor Anti-B, and, therefore, the subject has Group AB blood.

In the broadest sense, therefore, blood typing includes screening for RBC surface antigens, along with antibodies to RBC surface antigens. Technology that can identify the presence/absence of antigens (or receptors/binding sites) on cells and the presence/absence/concentration of antibodies (or possibly other molecules) in solution, is valuable in many fields of medical diagnostic screening and testing, pharmaceuticals, among others. Thus, the need exists for developing improved assays and methods for screening and blood typing, including forward typing, reverse grouping, antibody screening (e.g., IgG class antibodies), antibody identification, minor antigen typing, and extended phenotyping. Such assays and methods can also be generally applicable to other immunodiagnostics, such as infectious disease screening and allergy testing.

### SUMMARY

The present invention provides, at least in part, methods and devices for evaluating a sample (e.g., a plasma sample, a serum sample, or a whole blood sample), from a subject, for detecting a target molecule, e.g., an antibody (e.g., an antibody that binds to a red blood cell (RBC) antigen, a viral antigen, or a pathogenic antigen); or an antigen (e.g., an RBC antigen, a viral antigen, or a pathogenic antigen). In one embodiment, optimized antibody screening methods and devices are disclosed that significantly reduce the level of non-specific binding to a surface (e.g., a test surface bound with a red blood cell (rbcm) preparation), thus allowing for more efficient detection and reduced test time. In one embodiment, the optimized antibody screening method includes an immunoglobulin G (IgG) binding moiety that binds selectively and specifically to the plasma IgG present, relative to the binding to the lysed red blood cell membrane (rbcm) preparation. In another embodiment, an optimized antibody screening method is disclosed whereby non-specific binding caused by lysed red blood cell membrane preparations can be reduced by an agent that specifically cleaves a human IgG in the hinge region. In yet other embodiments, the invention provides methods and devices for target capturing that include a surface, e.g., a substantially planar surface, optionally having an optimized angle, for capture. Alternative solid phase geometries for capture are disclosed. Optimized methods for cell deposition are also disclosed. Thus, methods, devices, kits, and assays that include one or more of the aforesaid embodiments are disclosed.

The present invention can be applied to screening and blood typing, including, but not limited to, forward typing, reverse grouping, and antibody screening (e.g., IgG class antibody screening), antibody identification, minor antigen typing, and extended phenotyping. In other embodiments consistent with the present invention, the methods and devices disclosed herein are suitable for infectious disease screening (e.g., human immunodeficiency (HIV) virus, hepatitis B virus (HBV), syphilis, human T-lymphotropic virus (HTLV), hepatitis C virus (HCV), syphilis, among others), by testing for antibodies to these infectious agents, or, in some cases, testing for the agents themselves. In yet other embodiments, the invention can be applied to allergy testing (e.g., IgE antibody testing).

### Method of Detecting an Anti-RBC Antigen Antibody of G Isotype

Accordingly, in one aspect, the invention features a method of evaluating a sample (e.g., a plasma sample, a serum sample, or a whole blood sample), from a subject, for an anti-RBC-antigen antibody of G isotype. The method can be used, e.g., in antibody screening, antibody identification, or in pathogen analysis. The method includes:

(a) contacting a first red blood cell membrane preparation (an “rbcm preparation”) comprising a first RBC antigen, e.g., an Rh or Kell antigen, with the sample from the subject, under conditions sufficient for the formation of an immune complex between the first RBC antigen and the anti-first-RBC antigen antibody in the sample; and

(b) providing a detection reagent under conditions sufficient for the formation of a complex, e.g., an immune complex, between the detection reagent and an immunoglobulin G (IgG) antibody in the sample, said detection reagent comprising an IgG-specific binding moiety,

wherein the presence or absence of the anti-RBC antigen antibody in the sample is indicated by a value of a parameter, e.g., a measurable parameter, corresponding to the behavior of, or related to the positional distribution of, the detection reagent. E.g., a preselected value for a parameter related to the detection reagent, is indicative of the presence or absence of the anti-RBC antigen antibody. The parameter can be, by way of example, the amount of the detection reagent (e.g., an increased or decreased presence of the detection reagent); the pattern of coverage of the substrate by the detection reagent; the amount of coverage of the substrate by the detection reagent; the distribution of the detection reagent, e.g., on a substrate; the amount of aggregation of the detection reagent; the strength of adherence of the detection reagent, to the rbcm preparation (e.g., as detected by optical trapping), thereby evaluating a sample for an anti-RBC antigen antibody of G isotype.

In one embodiment, the IgG-specific binding moiety of the detection reagent used in the method is an antibody molecule (e.g., an antibody, e.g., a monoclonal antibody (mAb), or an antigen binding fragment thereof), having one or more of the following properties:

(i) it comprises mAb MS-278, or an antigen binding fragment thereof;

(ii) it competes with mAb MS-278 for binding to IgG;

(iii) it comprises at least one antigen binding region from mAb MS-278;

(iv) it comprises at least one, two or three complementarity determining regions (CDRs) from a heavy chain variable region of mAb MS-278;

(v) it comprises at least one, two or three CDRs from a light chain variable region of mAb MS-278;

(vi) it comprises a heavy chain variable region from mAb MS-278;

(vii) it comprises a light chain variable region from mAb MS-278;

(viii) it binds to an epitope bound by mAb MS-278;

(ix) it binds to rbcm preparations at a level, which is no more than 1.2, 1.5, 1.75, 2, 3, 4 or 5 times that of mAb MS-278, e.g., as determined by an assay described herein;

(x) it binds to IgG at a level which is at least 20, 30, 40, 50, 60, 70, 80, 90, or 100% of MS-278, e.g., as determined by an assay described herein;

(xi) when bound to rbcm preparation, e.g., as prepared as described herein, at least 20, 40, 60% of said binding is to IgG;

(xii) it binds to IgG with sufficient specificity that, under conditions described herein, it can distinguish between the presence and absence of a pre-selected anti-red blood cell antigen in less than 30, 25, 20, 15, 10, or 5 minutes;

(xiii) it is substantially free of binding to an rbcm preparation (e.g., an rbcm preparation described herein);

(xiv) its level of binding to a rbcm preparation is reduced by less than 10, 20, 30, 40, or 50% by pre-incubation of the rbcm preparation with an anti-IgG Fab or F(ab)₂ fragment;

(xv) its level of binding to a rbcm preparation is reduced by less than 10, 20, 30, 40, or 50% by pre-incubation of the rbcm preparation with an enzyme that alters or disrupts, e.g., cleaves, an IgG- or an IgG-like molecule (e.g., an IgG mimic), e.g., a cysteine proteinase with specificity for immunoglobulin G, such as an immunoglobulin-degrading enzyme of S. pyrogenes (IdeS), e.g., FabRICATOR®;

(xvi) its level of binding to a rbcm preparation is less than 1, 2, 5, 10, 25, or 50% of the binding of antibody chosen from 16H8 [Immucor], rabbit polyclonal [Alba #Z356], rabbit polyclonal [Biotest #804501], material from cell line CG-7 [Sigma-Aldrich I6260], or goat polyclonal [Sigma-Aldrich #I2136] to a rbcm preparation;

(xvii) it comprises an anti-IgG light chain antibody (mAb LCSIgG), e.g., an anti-light chain antibody chosen from Sigma-Aldrich #K4377 Cell Line KP-53, Sigma-Aldrich #L6522 cell line HP-6054, Sigma-Aldrich #K3502—polyclonal, or Sigma-Aldrich #L7646—polyclonal, or an antigen binding fragment thereof;

(xviii) it competes with the mAb LCSIgG for binding to IgG;

(xix) it binds to an epitope bound by the mAb LCSIgG; or

(xx) its level of binding to an rbcm preparation is less than 1, 2, 5, 10, 25, or 50% of the binding of mAb LCSIgG to a rbcm preparation, e.g., as described by an assay herein.

In one embodiment of the method, the IgG-specific binding moiety of the detection reagent comprises at least one antigen binding region, e.g., a variable region, from mAb MS-278. In one embodiment, the IgG-specific binding moiety comprises at least one or two variable region(s) from the heavy chain of mAb MS-278. In other embodiments, the IgG-specific binding moiety comprises at least one or two variable region(s) from the light chain of mAb MS-278. In one embodiment, the IgG-specific binding moiety comprises at least one or two variable region from the heavy chain, and at least one or two variable region(s) from the light chain, of mAb MS-278. In one embodiment, the IgG-specific binding moiety is a monomer of at least one or two variable region(s) from the heavy chain, and at least one or two variable region(s) from the light chain, of mAb MS-278. In other embodiments, the IgG-specific binding moiety is a dimeric, trimeric, tetrameric or pentameric form thereof. In one embodiment, the IgG-specific binding moiety is a pentamer of five monomers, each of which includes at least one or two variable region(s) from the heavy chain, and at least one or two variable region(s) from the light chain, of mAb MS-278. In one embodiment, the IgG-specific binding moiety is an IgM antibody that include at least one, two, three, four, five, six, seven, eight, nine, or ten heavy chain variable regions of mAb MS-278; and/or at least one, two, three, four, five, six, seven, eight, nine, or ten light chain variable regions of mAb MS-278. In one embodiment, the light chain variable region of mAb MS-278 comprises, consists essentially of, or consists of, the amino acid sequence of SEQ ID NO: 1, or an amino acid sequence substantially identical thereto (e.g., at least 80%, 85%, 90%, 95%, or 99% identical to the amino acid sequence of SEQ ID NO: 1, or which differs by at least 1 or 5 residues, but less than 40, 30, 20, or 10 residues from SEQ ID NO: 1). In one embodiment, the heavy chain variable region of mAb MS-278 comprises, consists essentially of, or consists of, the amino acid sequence of SEQ ID NO: 14, or an amino acid sequence substantially identical thereto (e.g., at least 80%, 85%, 90%, 95%, 99% identical to the amino acid sequence of SEQ ID NO: 14, or which differs by at least 1 or 5 residues, but less than 40, 30, 20, or 10 residues from SEQ ID NO: 14). In yet another embodiment, the heavy chain variable region of mAb MS-278 comprises, consists essentially of, or consists of, the amino acid sequence of SEQ ID NO: 39, or an amino acid sequence substantially identical thereto (e.g., at least 80%, 85%, 90%, 95%, 99% identical to the amino acid sequence of SEQ ID NO: 39, or which differs by at least 1 or 5 residues, but less than 40, 30, 20, or 10 residues from SEQ ID NO: 39).

In one embodiment of the method, the IgG-specific binding moiety comprises at least one, two or three CDRs from the light chain variable region of mAb MS-278. In one embodiment, the IgG-specific binding moiety comprises at least one, two or three CDRs from the heavy chain variable region of mAb MS-278. In one embodiment, the IgG-specific binding moiety comprises at least one, two or three CDRs from the light chain variable region, and at least one, two or three CDRs heavy chain variable regions of mAb MS-278. In one embodiment, the IgG-specific binding moiety is a monomer of at least one, two or three CDRs (e.g., CDRs 1-3) from the light chain variable region, and at least one, two or three CDRs (e.g., CDRs 1-3) from the heavy chain variable regions of mAb MS-278. In one embodiment, the IgG-specific binding moiety is a monomer comprising all six CDRs from MS-278. In other embodiments, the IgG-specific binding moiety is a dimeric, trimeric, tetrameric or pentameric form thereof. In one embodiment, the IgG-specific binding moiety is a pentamer of five monomers, each of which includes at least one, two or three CDRs (e.g., CDRs 1-3) from the light chain variable region, and at least one, two or three CDRs (e.g., CDRs 1-3) from the heavy chain variable regions of mAb MS-278.

In one embodiment, the light chain CDRs of mAb MS-278 comprise the amino acid sequence of SEQ ID NO: 2 (CDR1), SEQ ID NO: 3 (CDR2), or SEQ ID NOs: 4-12 (CDR3), or CDRs that have at least one amino acid alteration, but no more than two, three or four alterations (e.g., substitutions, deletions, or insertions (e.g., conservative substitutions)), compared to SEQ ID NOs:2-12. In one embodiment, the light chain CDR3 comprises the amino acid sequence, DPRT (SEQ ID NO:4) or SEQ ID NO:7. In other embodiments, the light chain CDR3 comprises the following consensus sequence:

X₁ X₂ X₃ X₄ X₅ X₆ D P R T (SEQ ID NO:5), wherein X₁=Q, A, G, or absent; X₂=A, G, F, Q, or absent; X₃=G, Q, P, Q, A or T; X₄=T, L or G; X₅=N, E or G; and X₆=E, N or V.

In other embodiments, the heavy chain CDRs of mAb MS-278 comprise the amino acid sequence of SEQ ID NOs: 15-21 (CDR1), SEQ ID NOs: 22-34 (CDR2), or SEQ ID NOs: 35-38 (CDR3), or CDRs that have at least one amino acid alteration, but no more than two, three or four alterations (e.g., substitutions, deletions, or insertions (e.g., conservative substitutions)), compared to compared to SEQ ID NOs:15-38. In one embodiment, the CDR3 comprises, consists essentially of, or consists of, the amino acid sequence of SEQ ID NO:38. In one embodiment, the heavy chain CDR1 comprises the following consensus sequence:

X₁ X₂ X₃S LS TSGMGVS (SEQ ID NO:15), wherein X₁ is G or Y; X₂ is F, G or Y; and X₃ is A or absent.

In one embodiment, the IgG-specific binding moiety comprises, consists essentially of, or consists of, a framework region (FR) (e.g., a region including at least FR1, FR2, FR3 and/or FR4) of mAb MS-278. In one embodiment, the framework region is a heavy chain variable framework region of mAb MS-278. In one embodiment, the heavy chain variable framework region comprises, consists essentially of, or consists of, at least FR1, FR2, FR3 and/or FR4 according to SEQ ID NO: 14 or 39, or an amino acid sequence substantially identical thereto (e.g., at least 80%, 85%, 90%, 95%, 99% identical to the amino acid sequence of SEQ ID NO: 14 or 39, or which differs by at least 1 or 5 residues, but less than 40, 30, 20, or 10 residues from SEQ ID NO: 14 or 39). In other embodiments, the framework region is a heavy chain variable framework region of mAb MS-278. In one embodiment, the light chain variable framework region comprises, consists essentially of, or consists of, at least FR1, FR2, FR3 and/or FR4 according to SEQ ID NO: 1, or an amino acid sequence substantially identical thereto (e.g., at least 80%, 85%, 90%, 95%, 99% identical to the amino acid sequence of SEQ ID NO: 1, or which differs by at least 1 or 5 residues, but less than 40, 30, 20, or 10 residues from SEQ ID NO: 1).

In yet other embodiments, the IgG-specific binding moiety comprises a light chain variable domain that comprises, consists essentially of, or consists of, one, two, or three CDRs including the following sequences:

RASESVDSYGNSFMH (SEQ ID NO:2) for CDR1, or a CDR that has at least one amino acid alteration, but no more than two, three or four alterations (e.g., substitutions, deletions, or insertions (e.g., conservative substitutions)), compared to compared to SEQ ID NO:2;

RASNLES (SEQ ID NO:3) for CDR2, or a CDR that has at least one amino acid alteration, but no more than two, three or four alterations (e.g., substitutions, deletions, or insertions (e.g., conservative substitutions)), compared to compared to SEQ ID NO:3; and/or

QQTNEDPRT (SEQ ID NO:7) for CDR3, or a CDR that has at least one amino acid alteration, but no more than two, three or four alterations (e.g., substitutions, deletions, or insertions (e.g., conservative substitutions)), compared to compared to SEQ ID NO:7.

In yet other embodiments, the IgG-specific binding moiety comprises a heavy chain variable domain that comprises, consists essentially of, or consists of, one, two, or three CDRs including the following sequences:

GFSLSTSGMGVS (SEQ ID NO:16) for CDR1, or a CDR that has at least one amino acid alteration, but no more than two, three or four alterations (e.g., substitutions, deletions, or insertions (e.g., conservative substitutions)), compared to compared to SEQ ID NO:16;

HIYWDDDKRYNPSLKS (SEQ ID NO:22) for CDR2, or a CDR that has at least one amino acid alteration, but no more than two, three or four alterations (e.g., substitutions, deletions, or insertions (e.g., conservative substitutions)), compared to compared to SEQ ID NO:22; and/or

ARSDGYYHYAMLDY (SEQ ID NO:38) for CDR3, or a CDR that has at least one amino acid alteration, but no more than two, three or four alterations (e.g., substitutions, deletions, or insertions (e.g., conservative substitutions)), compared to compared to SEQ ID NO:38.

In certain embodiments, the IgG-specific binding moiety comprises:

a light chain variable domain that comprises, consists essentially of, or consists of, one, two, or three CDRs including the following sequences:

RASESVDSYGNSFMH (SEQ ID NO:2) for CDR1, or a CDR that has at least one amino acid alteration, but no more than two, three or four alterations (e.g., substitutions, deletions, or insertions (e.g., conservative substitutions)), compared to compared to SEQ ID NO:2;

RASNLES (SEQ ID NO:3) for CDR2, or a CDR that has at least one amino acid alteration, but no more than two, three or four alterations (e.g., substitutions, deletions, or insertions (e.g., conservative substitutions)), compared to compared to SEQ ID NO:3; and/or

QQTNEDPRT (SEQ ID NO:7) for CDR3, or a CDR that has at least one amino acid alteration, but no more than two, three or four alterations (e.g., substitutions, deletions, or insertions (e.g., conservative substitutions)), compared to compared to SEQ ID NO:7; and a heavy chain variable domain that comprises, consists essentially of, or consists of, one, two, or three CDRs including the following sequences:

GFSLSTSGMGVS (SEQ ID NO:16) for CDR1, or a CDR that has at least one amino acid alteration, but no more than two, three or four alterations (e.g., substitutions, deletions, or insertions (e.g., conservative substitutions)), compared to compared to SEQ ID NO:16;

HIYWDDDKRYNPSLKS (SEQ ID NO:22) for CDR2, or a CDR that has at least one amino acid alteration, but no more than two, three or four alterations (e.g., substitutions, deletions, or insertions (e.g., conservative substitutions)), compared to compared to SEQ ID NO:22; and/or

ARSDGYYHYAMLDY (SEQ ID NO:38) for CDR3, or a CDR that has at least one amino acid alteration, but no more than two, three or four alterations (e.g., substitutions, deletions, or insertions (e.g., conservative substitutions)), compared to compared to SEQ ID NO:38.

In other embodiments, the IgG-specific binding moiety comprises a light chain variable domain that comprises, consists essentially of, or consists of, the amino acid sequence of SEQ ID NO:1, or an amino acid sequence substantially identical thereto (e.g., at least 80%, 85%, 90%, 95%, 99% identical to the amino acid sequence of SEQ ID NO: 1, or which differs by at least 1 or 5 residues, but less than 40, 30, 20, or 10 residues from SEQ ID NO: 1).

In yet other embodiments, the IgG-specific binding moiety comprises a heavy chain variable domain that comprises, consists essentially of, or consists of, the amino acid sequence of SEQ ID NO:39, or an amino acid sequence substantially identical thereto (e.g., at least 80%, 85%, 90%, 95%, 99% identical to the amino acid sequence of SEQ ID NO: 39, or which differs by at least 1 or 5 residues, but less than 40, 30, 20, or 10 residues from SEQ ID NO: 39).

In yet another embodiment, the IgG-specific binding moiety comprises:

a light chain variable domain that comprises, consists essentially of, or consists of, the amino acid sequence of SEQ ID NO:1, or an amino acid sequence substantially identical thereto (e.g., at least 80%, 85%, 90%, 95%, 99% identical to the amino acid sequence of SEQ ID NO: 1, or which differs by at least 1 or 5 residues, but less than 40, 30, 20, or 10 residues from SEQ ID NO: 1); and

a heavy chain variable domain that comprises, consists essentially of, or consists of, the amino acid sequence of SEQ ID NO:39, or an amino acid sequence substantially identical thereto (e.g., at least 80%, 85%, 90%, 95%, 99% identical to the amino acid sequence of SEQ ID NO: 39, or which differs by at least 1 or 5 residues, but less than 40, 30, 20, or 10 residues from SEQ ID NO: 39).

In one embodiment, the detection reagent further comprises an indicator moiety, e.g., a red blood cell, and (optionally) one or more binding agents, e.g., IgG-specific binding agents. In one embodiment, the detection reagent includes an IgG-sensitized red blood cell. In such embodiments, a base unit (or unit) of the detection reagent comprises the indication moiety, e.g., the red blood cell, optionally, containing the binding agents. In certain embodiments, base units of the detection are capable of complexing to form aggregates.

In another embodiment of the method, less than 1, 2, 3, 5, 10, 15, 20, or 25% (and more typically, less than 5, 10, 15, 20, or 25%) of the IgG-sensitized red blood cells bind, e.g., as determined by optical trapping, to the rbcm preparation that has been incubated with the IgG-specific binding moiety, e.g., an IgG-specific antibody or antigen binding fragment thereof, e.g., as determined by a method described herein.

In one embodiment of the method, the IgG-specific binding moiety, e.g., an IgG-specific antibody or antigen binding fragment thereof, binds IgG at a pre-selected concentration of IgG. E.g., it provides a pre-selected limit of detection, e.g., a limit of detection described herein.

In another embodiment of the method, the IgG-specific binding moiety, e.g., an IgG-specific antibody or antigen binding fragment thereof, has the following property:

when plasma containing an antibody to a first red blood cell antigen is incubated with an rbcm preparation that includes the first red blood cell antigen (the positive preparation) and with rbcm preparation which does not include the first red blood cell antigen (the negative preparation), at least 15, 20, 25, or 30% of red blood cells functionalized with the IgG-specific binding moiety, e.g., an IgG-specific antibody or antigen binding fragment thereof, show specific binding, and less than 5 or 10% show non-specific binding, e.g., as determined by a method described herein. In an embodiment, the specific binding increases over time, e.g., over 4, 5, 6, or 7 minutes, to at least 40%, while the nonspecific signal increases by less than 20%, e.g., to a control preparation, e.g., a negative preparation, as determined by a method described herein.

In yet another embodiment of the method, the IgG-specific binding moiety is an anti-light chain antibody, and displays specific binding of at least 10, 20, or 30% and non specific binding of less than 2 or 5%, e.g., as determined by a method described herein.

In certain embodiments, the rbcm preparation is disposed on a surface to form a substrate. In one embodiment, the rbcm preparation is bound (e.g., non-covalently or covalently) to a surface, e.g., a functionalized surface. For example, an rbcm preparation containing pre-selected red blood cells can be disposed (e.g., by centrifugation or gravitational settling) onto a surface capable of binding red blood cells. In embodiments, the rbcm preparation provides a substrate having a density of between 14000-24000, 24000-34000 and 34000-40000, cells/mm², e.g., 26,000 cells/mm² on the surface. Protocols and exemplary surfaces to be used in the methods are described herein below.

As described above, the detection reagent can include a red blood cell as an indicator moiety. Such detection reagents may (optionally) include one or more binding agents, e.g., IgG-specific binding agents. In one embodiment, the detection reagent includes an IgG-sensitized red blood cell.

In one embodiment, the detection reagent is present at a concentration that results in less than the entire substrate being covered with a monolayer. E.g., the detection reagent is present in an amount that provides a sparse coating of the substrate. In embodiments, the detection reagent is present in an amount that results in coverage of less than or about 5%, 10%, 15%, 20%, 25% or 30% of the area of the substrate.

In other embodiments, the concentration of detection reagent is such that at least 30, 40, 50, 60, 70, 80, 90, or 100% of the substrate is covered with at least a monolayer of the detection reagent. In embodiments, the detection reagent is present at a concentration that results in the entire substrate being covered with at least a monolayer. In embodiments portions of the substrate are covered with more than one layer of the detection reagent, e.g., portions of the substrate are covered by multilayer of detection reagent. In embodiments, the detection reagent is present in an amount that is at least 10, 20, 30, 40, 50, 60, 70, 80, 90, or 100, and typically at least 50 times the amount that would give 20% coverage of the substrate with a monolayer.

In certain embodiments, the presence or absence of the anti-RBC antigen antibody in the sample is indicated by a value for a parameter, e.g., a measurable parameter, corresponding to the behavior of, or related to the positional distribution of, the detection reagent. E.g., a preselected value for a parameter related to indicator moieties, e.g., indicator cells, is indicative of the presence or absence of the anti-RBC antigen antibody. The parameter can be, by way of example, the amount of the indicator moieties, e.g., indicator cells (e.g., an increased or decreased presence of the indicator moiety, e.g., indicator cell); the pattern of coverage of the substrate by the indicator moieties, e.g., indicator cells; the amount of coverage of the substrate by the indicator moieties, e.g., indicator cells; the distribution of the indicator moieties, e.g., indicator cells, e.g., on a substrate; the amount of aggregation of the indicator moieties, e.g., indicator cells; the strength of adherence of the indicator moieties, e.g., indicator cells, to the rbcm preparation (e.g., as detected by optical trapping).

In one embodiment, the difference in the detection reagent includes in one or more of: a difference in the amount of the detection reagent (e.g., an increased or decreased presence of the detection reagent); a difference in the pattern of coverage of the substrate by the detection reagent; a difference in the amount of coverage of the substrate by the detection reagent; a difference in the distribution of the detection reagent, e.g., on a substrate; a difference in the amount of aggregation of the detection reagent; or a difference in the strength of adherence of the detection reagent to the rbcm preparation (e.g., as detected by optical trapping).

In certain embodiments, the presence or absence of the anti-RBC antigen antibody in the sample is indicated by a parameter related to the indicator moieties, e.g., indicator cells.

In one embodiment, the presence of the anti-RBC antigen antibody in the sample (or a positive readout) is detected by a uniform, homogenous distribution of the detection reagent on the substrate. In one embodiment, the positive readout is detected by having a coverage of the substrate by the detection reagent of at least 90%, 95%, 96%, 97%, 98%, 99% or 100% of the substrate area. An exemplary representation of a uniform distribution of the detection reagent is provided in FIG. 16C.

In another embodiment, the absence of the anti-RBC antigen antibody in the sample (or a negatve read out) is detected by a non-homogeneous distribution of the detection reagent on the substrate. In one embodiment, the negative readout is detected by having a coverage of the substrate by the detection reagent of less than 99.9%, 99%, 95%, 90%, 85%, 80%, 75%, 70%, 60%, 50%, 40% or 30% of the substrate area (e.g., relative to what would be covered in a positive sample). An exemplary representation of a non-homogeneous distribution of the detection reagent is provided in FIG. 16D. In one embodiment, the negative readout is detected as a localized concentration of the detection reagent, e.g., as a button or a pellet.

In certain embodiments, the difference in the detection reagent is detected by an increased or decreased formation of an aggregate.

In one embodiment, base units of non-bound detection reagent (detection reagent not bound to the rbcm preparation) form detection reagent complexes with one another, e.g., to form aggregates of non-bound detection reagent. In embodiments, said aggregate comprises at least 2, 10, 20, 50, 100, 200, 1,000, 100,000, 1,000,000, 10,000,000 or 50,000,000 base units of detection reagent. In one embodiment, the aggregate is of macroscopic dimension, e.g., an aggregate having an average dimension, e.g., at its largest point, of between 10-500 um, 75 um-1 mm, 100 um and 10 mm.

In one embodiment, non-bound detection reagent, e.g., detection reagent complexes, e.g., an aggregate, is separated from detection reagent bound to an anti-RBC antibody, which anti-RBC antibody is bound to said first rbcm preparation (e.g., detection reagent in an immune complex with an said RBC antigen on said first rbcm preparation).

In an embodiment of the method, detection reagent unit traverses the substrate and collides with a second (or subsequent) detection reagent unit, e.g., a detection reagent unit that traverses more slowly or is bound.

In one embodiment of the method, the detection reagent, e.g., detection reagent complexes, e.g., an aggregate, that fails to bind to said first rbcm preparation migrates across a substrate, e.g., into said first negative readout region of said carrier.

In other embodiments, the method further includes providing sufficient conditions, e.g., tangential velocity and sufficient time for a detection reagent, e.g., detection reagent complexes, e.g., an aggregate, that has not formed an immune complex to migrate across the substrate. In an embodiment, this results in uncovering substrate or reducing the amount of substrate covered by detection reagent. In embodiments, the aggregate can migrate a first negative readout region.

In another embodiment, the difference in the detection reagent is detected by evaluating the strength of adherence of the detection reagent to the rbcm preparation, e.g., to the substrate (e.g., as detected by optical trapping). In one embodiment, the displacement of non-bound detection reagent is evaluated by the optical trapping.

In one embodiment of the method, the presence or absence of detection reagent complexes, e.g., an aggregate, e.g., in a pre-selected location, is correlated with, respectively, the absence or presence, of said anti-RBC antigen antibody in said sample.

In another embodiment of the method, the presence, absence, or amount of detection reagent complexes, e.g., an aggregate, is detected in a readout region. In one embodiment, the readout region is on the rbcm preparation.

In one embodiment of the method, the detection of the presence of detection reagent complexes, e.g., an aggregate, e.g., in said readout region, is correlated with the absence or the presence of said anti-RBC antigen antibody in said sample.

The readout region can be disposed in a chamber, e.g., a well or tube.

In one embodiment of the method, said first rbcm preparation is disposed on a carrier and the presence of detection reagent that is not in detection reagent complexes, e.g., an aggregate, e.g., in a first positive readout region, of said carrier is positively correlated with the presence of an anti-first RBC antigen antibody in said sample. In another embodiment, the presence of detection reagent, e.g., detection reagent complexes, e.g., an aggregate, e.g., in a first negative readout region disposed on said carrier, or on another carrier, is negatively correlated with the presence of an anti-first RBC-antigen antibody in said sample.

In certain embodiment, the detection reagent, e.g., detection reagent complexes, e.g., an aggregate, that has not formed an immune complex migrates from said positive readout region into said negative readout region.

In other embodiments, a detection reagent which has not formed an immune complex or a detection reagent complex does not migrate to negative readout region, but detection reagent which has not formed an immune complex but has formed a detection reagent complex, e.g., a macroscopic reagent complex, migrates to a negative readout region.

In other embodiments, the first positive readout region and first negative readout regions are spatially distinct, e.g., separated, on said carrier. In one embodiment, the first readout region is disposed in a chamber, e.g., a well or tube. In another embodiment, the first negative readout region is disposed in a chamber, e.g., a well or tube. In other embodiments, the first negative readout region and a first positive readout region are disposed in a chamber, e.g., a well or tube.

In other embodiments, the method includes:

contacting said first rbcm preparation with sample from said subject under conditions sufficient for the formation of an immune complex between said first RBC antigen and anti-first RBC antigen antibody to form a first reaction mixture;

contacting said first reaction mixture with said detection reagent under conditions sufficient for the formation of an immune complex between said detection reagent and an IgG antibody in said sample,

allowing sufficient time for detection reagent that has not formed an immune complex be detected, e.g., by detection of detection reagent complexes, e.g., an aggregate.

In yet other embodiments, the method further includes:

(c) contacting a second rbcm preparation comprising a second RBC antigen, e.g., a Duffy antigen, and optionally, being substantially free of said first pre-selected RBC antigen, with sample from said subject under conditions sufficient for the formation of an immune complex between said second RBC antigen and anti-second RBC antigen antibody in said sample;

(d) providing detection reagent under conditions sufficient for the formation of a complex, e.g., an immune complex, between said detection reagent and an IgG antibody in said sample,

optionally, wherein said first blood cell membrane preparation is substantially free of said second RBC antigen, and

wherein the presence or absence of said detection reagent, e.g., in a preselected location, is correlated with the presence or absence of said anti-second RBC antigen antibody in said sample, thereby evaluating a sample for an anti-second RBC antigen antibody of G isotype.

In other embodiments of the method, the method includes antibody identification and a second rbcm preparation comprising a second RBC antigen that is substantially free of said first pre-selected RBC antigen, and said first blood cell membrane preparation is substantially free of said second RBC antigen.

In certain embodiments, steps (a) and (c) of the methods are performed at least partially simultaneously. In other embodiments, steps (b) and (d) of the methods are performed at least partially simultaneously.

In other embodiments of the methods, said second rbcm preparation is spatially distinct, e.g., separate, from said first rbcm preparation.

In other embodiments, the method further includes evaluating said sample for an N^{th}, e.g., third, anti-RBC antigen antibody of IgG isotype by:

(e) contacting an N^{th}, e.g., third, rbcm preparation comprising an N^{th}, e.g., third, pre-selected RBC antigen (and optionally being substantially free of at least one or more or all other antigens tested for) with sample from said subject, under conditions sufficient for the formation of an immune complex between said N^{th}, e.g., third, RBC antigen and anti-N^{th}, e.g., third, RBC antigen antibody in said sample,

(f) providing detection reagent under conditions sufficient for the formation of a complex, e.g., an immune complex, between said detection reagent and an antibody of said pre-selected isotype in said sample,

optionally, wherein one, some or all of said N−1th first blood cell membrane preparations are substantially free of said Nth, e.g., third, pre-selected-RBC antigen and

wherein the presence or absence of said detection reagent, e.g., in a preselected location, is correlated with the presence or absence of said anti-N^{th}, e.g., third, RBC antigen antibody in said sample,

thereby evaluating a sample for an anti-N^{th}, e.g., third, RBC antigen antibody.

In certain embodiments, steps (a) and (e) of the methods are performed at least partially simultaneously. In other embodiments, steps (b) and (f) of the methods are performed at least partially simultaneously.

The methods of the invention can be used to evaluate a sample, e.g., a plasma sample, from said subject for an antibody to at least 1, 2, 3, 4, or all of the RBC antigens provided in Table 1. In one embodiment, the method includes evaluating sample from said subject for an antibody to at least 1, 2, 3, 4, or all of the following RBC antigens: a Rhesus antigen, e.g., one or more or all of D, C, c, E, or e; an MNS antigen, e.g., one or more or all of M, N, S, or s; a Kidd antigen, e.g., one or both of Jk^{a} or Jk^{b}; a Duffy antigen, e.g., one or both of Fy^{a} or Fy^{b}; a Kell antigen, e.g., one or both of K or k; a Lewis antigen, e.g., one or both of Le^{a} or Le^{b}; or a P antigen, e.g., P1. In certain embodiments, the method includes evaluating sample from said subject for an antibody to at least the following RBC antigens: (1) D, C, E, e, c, and K; (2) D, C, E, e, c, K, Fy^{a} and Jk^{a}; and (3) D, C, E, e, c, K, Fy^{a}, Fy^{b}, Jk^{a}, Jk^{b}, S, and s.

In certain embodiments, the method includes at least X rbcm preparations, wherein each of the antigens listed above is present in at least one of said preparations and absent from another, wherein X=2, 3, 4, 5, 10, 15 or 20. For example, the sample from said subject with a panel of rbcm preparations includes: a first rbcm preparation comprising RBC antigen K and being substantially free of RBC antigens D and Fy^{a}; a second rbcm preparation comprising RBC antigen Fy^{a} and being substantially free of RBC antigens K and D; and a third rbcm preparation comprising RBC antigen D and being substantially free of RBC antigens Fy^{a} and K.

In other embodiments, the method includes:

providing a panel comprising a plurality of rbcm preparations disposed on a surface or carrier, each rbcm preparation of said plurality being spatially distinct, e.g., separated, from the other rbcm preparations on said surface or carrier;

contacting a plurality of said rbcm preparations with sample from said subject under conditions sufficient for the formation of an immune complex between a RBC antigen and anti-RBC antigen antibody in said sample, to form a plurality of first reaction mixtures;

contacting each of said plurality of first reaction mixtures with a detection reagent under conditions sufficient for the formation of an immune complex between said detection reagent and an IgG from said sample, to form a plurality of second reaction mixtures; and

for each of a plurality of said second reaction mixtures, allowing sufficient time for detection reagent that has not formed an immune complex to form detection reagent complexes, e.g., to aggregate; to migrate into a negative readout region; or, to form detection reagent complexes, e.g., to aggregate, and migrate into a negative readout region; wherein the formation of detection reagent complexes, e.g., an aggregate; the presence or absence of said detection reagent in a negative readout region; the formation of detection reagent complexes, e.g., an aggregate, in said negative readout region, is correlated with the presence or absence an anti-blood-type-antigen antibody in said sample.

In other embodiments, the sample is incubated with said rbcm preparation in an incubation phase; optionally, said rbcm preparation is washed; detection reagent is added; and said incubated rbcm preparation is centrifuged to allow formation of detection reagent complexes, e.g., aggregates, of base units of unbound detection reagent, in a readout phase. In one embodiment, the duration of said readout phase is 1-6, 2-4, 1-2, e.g., 3, or less, minutes. In other embodiments, the duration of said incubation phase is 1-8, 2-7, 3-6, e.g., 5, or less, minutes. In yet other embodiments, the duration of said incubation and readout phases is 2-15, 5-15, 10-15, e.g., 12, or less, minutes.

In another embodiment, the rbcm preparation (e.g., the first rbcm preparation) is disposed on a surface, e.g., a substantially planar surface or substrate, and the angle between said surface, e.g., a substantially planar surface or substrate, and the direction of applied force, e.g., centrifugal, gravitational, fluid magnetic, electric or fluid, force, that causes migration, e.g., sedimentation, of detection reagent, is other than 90 degrees.

In other embodiments, the method further includes forming a rbcm preparation from a sample comprising red blood cells. In one embodiment, the method includes disposing first rbcm preparation on said carrier. In other embodiments, the method includes lysing red blood cells in said sample comprising red blood cells.

In other embodiments, the method includes contacting the sample from said subject with anti-first first-blood-type-antigen to evaluate the presence of said first-blood-type-antigen in said subject.

In one embodiment, the subject is a donor of an organ or tissue, e.g., blood. In another embodiment, the subject is a recipient of an organ or tissue, e.g., blood.

Alternatively, or in combination with the methods described herein, said rbcm preparation is contacted with an agent, e.g., an enzyme, e.g., IdeS (immunoglobulin G-degrading enzyme of S. pyrogenes), e.g., FabRICATOR®, that cleaves a protein, e.g., an IgG- or an IgG-like molecule, e.g., an IgG mimic. In such embodiments, the invention provides a method of evaluating a sample, e.g., a plasma sample, from a subject, for an anti-RBC-antigen antibody of G isotype. The method includes:

(a) contacting a first red blood cell membrane preparation (a rbcm preparation) comprising a first RBC antigen, e.g., an Rh or Kell antigen, with an agent that alters or cleaves an IgG- or an IgG-like molecule, e.g., an IgG mimic; thereby forming an optimized rbcm preparation;

(b) contacting the optimized rbcm preparation with a sample from the subject, under conditions sufficient for the formation of an immune complex between said first RBC antigen and anti-first-RBC antigen antibody in said sample; and

(c) providing a detection reagent under conditions sufficient for the formation of a complex, e.g., an immune complex, between said detection reagent and an IgG antibody in said sample, said detection reagent comprising an IgG-specific binding moiety,

wherein the presence or absence of the anti-RBC antigen antibody in the sample is indicated by a value of a parameter, e.g., a measurable parameter, corresponding to the behavior of, or related to the positional distribution of, the detection reagent. E.g., a preselected value for a parameter related to the detection reagent, is indicative of the presence or absence of the anti-RBC antigen antibody. The parameter can be, by way of example, the amount of the detection reagent (e.g., an increased or decreased presence of the detection reagent); the pattern of coverage of the substrate by the detection reagent; the amount of coverage of the substrate by the detection reagent; the distribution of the detection reagent, e.g., on a substrate; the amount of aggregation of the detection reagent; the strength of adherence of the detection reagent, to the rbcm preparation (e.g., as detected by optical trapping), as described herein.

In one embodiment, the agent is an enzyme, e.g., a cysteine proteinase with specificity for immunoglobulin G. In one embodiment, the enzyme is an immunoglobulin-degrading enzyme of S. pyrogenes (IdeS), e.g., FabRICATOR®.

The invention additionally provides optimized or mimic-optimized rbcm preparation, e.g., made by the methods described herein.

In yet another aspect, the invention features a method of evaluating a sample, e.g., a plasma sample, from a subject, for an anti-RBC-antigen antibody of a G isotype (IgG antibody), comprising:

(a) contacting a first mimic-optimized red blood cell membrane preparation (a mo-rbcm preparation) comprising a first RBC antigen, e.g., an Rh or Kell antigen, with sample from said subject, under conditions sufficient for the formation of an immune complex between said first RBC antigen and anti-first-RBC antigen antibody in said sample; and

(b) providing a detection reagent under conditions sufficient for the formation of a complex, e.g., an immune complex, between said detection reagent and an IgG antibody in said sample, said detection reagent comprising an IgG binding moiety, e.g., an IgG binding moiety is an IgG-specific binding moiety as described herein,

wherein the presence or absence of the anti-RBC antigen antibody in the sample is indicated by a value of a parameter, e.g., measurable parameter, corresponding to the behavior of, or related to the positional distribution of, the detection reagent. E.g., a preselected value for a parameter related to the detection reagent, is indicative of the presence or absence of the anti-RBC antigen antibody. The parameter can be, by way of example, the amount of the detection reagent (e.g., an increased or decreased presence of the detection reagent); the pattern of coverage of the substrate by the detection reagent; the amount of coverage of the substrate by the detection reagent; the distribution of the detection reagent, e.g., on a substrate; the amount of aggregation of the detection reagent; the strength of adherence of the detection reagent, to the rbcm preparation (e.g., as detected by optical trapping) as described herein,

thereby evaluating a sample for an anti-RBC antigen antibody of G isotype.

In one embodiment, the mimic-optimized-rbcm preparation is a rbcm preparation that has been contacted with a proteolytic enzyme, e.g., IdeS (immunoglobulin G-degrading enzyme, e.g., of S. pyrogenes), e.g., FabRICATOR®.

### Methods of Evaluating a Sample for a Red Blood Cell Antigen

In another aspect, the invention features a method of evaluating a sample for a red blood cell antigen, e.g., forward typing, minor antigen typing, or extended phenotyping, comprising:

(a) contacting a red blood cell antigen binding agent, e.g., an anti-red blood cell antigen antibody, disposed on a surface (e.g., a functionalized surface as described herein) with the sample, e.g., a sample containing one or more red blood cells, under conditions sufficient for the formation of a complex between said red blood cell antigen binding agent, e.g., anti-red blood cell antigen antibody, and a red blood cell in said sample to occur, wherein said red blood cell comprises the red blood cell antigen (referred to herein as “complexed cells”);

(b) separating the complexed cells, e.g., by causing differential migration of red blood cells not complexed with said red blood cell antigen binding agent, e.g., anti-red blood cell antigen antibody (“uncomplexed cells”), relative to the complexed cells, across said substrate, wherein a change, e.g., an increase or decrease, in the amount of complexed and/or uncomplexed red blood cells, is correlated with the amount of said red blood cell antigen in said sample, thereby evaluating a sample for a red blood type antigen.

In an embodiment, the red blood cell antigen is a blood-type antigen, e.g., an A, B, or AB antigen.

In an embodiment, the red blood cell antigen is a blood-type antigen, e.g., a D antigen.

In one embodiment, the method is a forward typing method, e.g., comprises the detection of a red blood cell antigen chosen from an A, B, or D antigen.

In an embodiment the red blood cell antigen is chosen from at least 1, 2, 3, 4, 5, 6, 7, 8, 9, 10, 11, 12 or all of the RBC antigens provided in Table 1.

In one embodiment, the red blood cell antigen is a minor antigen.

In one embodiment, the red blood cell antigen is chosen from one, two, three, four, five, six, seven, eight, nine, ten, eleven, twelve, or more, or all of:

a Rhesus antigen, e.g., one or more or all of D, C, c, E, or e;

a MNS antigen, e.g., one or more or all of M, N, S, or s;

a Kidd antigen, e.g., one or both of Jk^{a} or Jk^{b};

a Duffy antigen, e.g., one or both of Fy^{a} or Fy^{b};

a Kell antigen, e.g., one or both of K or k;

a Lewis antigen, e.g., one or both of Le^{a} or Le^{b}; or

P antigen, e.g., P1.

In certain embodiments, the red blood cell antigen analyzed includes at least the following RBC antigens: (1) D, C, E, e, c, and K; (2) D, C, E, e, c, K, Fy^{a} and Jk^{a}; or (3) D, C, E, e, c, K, Fy^{a}, Fy^{b}, Jk^{a}, Jk^{b}, S, and s.

In one embodiment, the red blood cell antigen binding agent is a molecule that binds to a red blood cell antigen, e.g., a protein, a peptide or a carbohydrate. In one embodiment, the red blood cell antigen binding agent is an anti-red blood cell antigen antibody (e.g., an IgG or an IgM, or a combination thereof). In other embodiments, the red blood cell antigen binding agent is a plant-derived binding agent, e.g., a lectin.

In an embodiment, the change, e.g., presence or absence, of detection uncomplexed cells is detected by in one or more of: a difference in the amount of the detection reagent (e.g., an increased or decreased presence of the detection reagent); a difference in the distribution of the detection reagent, e.g., on a surface; a difference in the amount of aggregation of the detection reagent; or a difference in the strength of adherence of the detection reagent to the rbcm preparation (e.g., as detected by optical trapping).

In one embodiment, the separation is effected by applying acceleration, e.g., centrifugal, fluid magnetic, electric or fluid, that causes migration of the complexed and uncomplexed cells.

In an embodiment, the surface is configured such that the applied acceleration results in migration of uncomplexed cells into a defined region, e.g., at the bottom of a chamber (e.g., a well or a tube). In an embodiment, the detection of the presence of uncomplexed cells (e.g., a negative readout) is correlated with the absence of binding between said anti-RBC antigen antibody in said sample. In certain embodiments, the negative readout is a button or a pellet. Exemplary schematics of negative readouts are shown in FIGS. 1D and 3D as samples E and F.

In one embodiment, the detection of the presence of complexed cells (e.g., a positive readout) is correlated with the presence of binding between said anti-RBC antigen antibody in said sample. In certain embodiments, the positive readout is detected as a haze. A schematic of the top views of the readout in chamber is depicted in FIGS. 1D and 3D, where a positive readout is detected as a haze in sample D of FIGS. 1D and 3D.

Representative images of positive and negative readouts of some of the forward typing assays described herein are shown in FIGS. 1E-1G. For example, FIGS. 1E and 1G provide a representative image of a positive readout showing a ‘haze’ of blood cells in the sample, which indicates that binding between the sample blood cells and the surface has occurred. FIG. 1F provides an image of a representative negative readout showing a pellet of red blood cells, which indicates that binding between the sample red blood cells and the surface has not occured.

In an embodiment, the readout region is disposed in a chamber, e.g., a well or tube.

In an embodiment, the chamber is disposed on a carrier, e.g., a multi-chamber or multi-well plate, e.g., a 96 well plate.

In an embodiment, the angle between said carrier and the direction of force is normal, e.g., 0 degrees.

In an embodiment, the angle between said carrier and the direction of force is non normal, e.g., between 25-5, 20-7.5, or 10 degrees.

In certain embodiments, the anti-red blood cell antigen antibody is an IgG, an IgM or a combination thereof. In an embodiment, the red blood cell binding agent, e.g., antigen antibody, is disposed on the inner surface of a chamber, e.g., well or tube (e.g., as depicted by the side views of the chambers for forward typing shown in FIGS. 1A-1C; or the extended phenotypic chambers depicted in FIGS. 3A-3C).

### Methods of Evaluating a Sample for a Red Blood Cell Antigen-Specific Antibody

In another aspect, the invention features a method of evaluating a sample for a red blood cell (RBC) antigen-specific antibody, e.g., reverse grouping or typing. The method comprises:

(a) contacting a rbcm preparation which specifically presents or lacks certain red blood cell antigens, e.g., A− cells presenting the A antigen and not the B antigen, B− cells presenting the B antigen and not the A antigen, and O− cells presenting neither the A antigen or B antigen, disposed as a substrate of a surface, with sample, under conditions sufficient for the formation of a complex between said rbcm preparation and an anti-red blood cell antigen-specific antibody, e.g., anti-A or anti-B antibody, in said sample;

(b) providing indicator moieties, e.g., one or more indicator cells which specifically presents or lacks said red blood cell antigen (e.g., A+, B+, or O+ indicator cells), under conditions sufficient for the formation of a complex, e.g., an immune complex, between said rbcm preparation and the indicator moieties, e.g., the indicator cells;

(c) providing an agent (e.g., a multi-valent binding agent, e.g., anti-D antibody of M isotype) that can promote clumping between the indicator moieties, e.g., the indicator cells, under conditions sufficient for the formation of a complex, e.g., an immune complex, of said indicator moieties, via said agent (e.g., the multi-valent binding agent),

(d) applying an acceleration, e.g., from a centrifugal, a gravitational, a fluid magnetic, an electric or a fluid, force,

wherein said indicator moieties, e.g., by the distribution of indicator moieties, or by the strength of adhesion of unbound indicator moieties to the substrate, indicate the presence or absence of said red blood cell antigen-specific antibody,

thereby evaluating said sample.

In an embodiment, the indicator moiety is a red blood cell.

In an embodiment, the multi-valent binding agent, e.g., an IgM antibody, binds a moiety that is present on said indicator cells, but not present on said rbcm. In an example, the moiety is a red blood cell antigen other than the red blood cell antigen being analysed. In an embodiment, the moiety is other than a blood group antigen. In an embodiment, the moiety is D antigen, and the multi-valent binding agent, e.g., an IgM antibody, is an anti-D antibody. In an embodiment, the rbcm preparation is negative for D antigen and the indicator cells are positive for D antigen.

In certain embodiments, the rbcm preparation is disposed on a surface to form a substrate. In one embodiment, the rbcm preparation is bound (e.g., non-covalently or covalently) to a surface, e.g., a functionalized surface. For example, an rbcm preparation containing pre-selected red blood cells can be disposed (e.g., by centrifugation or gravitational settling) onto a surface capable of binding red blood cells. In embodiments, the rbcm preparation provides a substrate having a density of between 14000-24000, 24000-34000 and 34000-40000, cells/mm², e.g., 26,000 cells/mm² on the surface. Protocols and exemplary surfaces to be used in the methods are described herein below.

In one embodiment, the indicator moieties, e.g., the indicator cells, are present at a concentration that results in less than the entire substrate being covered with a monolayer of indicator moieties, e.g., indicator cells. E.g., the indicator moieties, e.g., the indicator cells, are present in an amount that provides a sparse coating of the substrate. In embodiments, the indicator moieties, e.g., indicator cells, are present in an amount that results in coverage of less than or about 5%, 10%, 15%, 20%, 25% or 30% of the area of the substrate.

In other embodiments, the concentration of indicator moieties, e.g., indicator cells, is such that at least 30, 40, 50, 60, 70, 80, 90, or 100% of the substrate is covered with at least a monolayer of indicator moieties. In embodiments, the indicator moieties are present at a concentration that results in the entire substrate being covered with at least a monolayer. In embodiments portions of the substrate are covered with more than one layer of indicator moieties, e.g., portions of the substrate are covered by a multilayer of indicator moieties. In embodiments, the detection indicator moieties, e.g., indicator cells, are present in an amount that is at least 10, 20, 30, 40, 50, 60, 70, 80, 90, or 100, and typically at least 50 times the amount that would give 20% coverage of the substrate with a monolayer.

In certain embodiments, the presence or absence of the anti-RBC antigen antibody in the sample is indicated by a parameter, e.g., a measurable parameter, related to the behavior or positional distribution of indicator moieties, e.g., indicator cells. E.g., a preselected value for a parameter related to indicator moieties, e.g., indicator cells, is indicative of the presence or absence of the anti-RBC antigen antibody. The parameter can be, by way of example, the amount of the indicator moieties, e.g., indicator cells (e.g., an increased or decreased presence of the indicator moiety); the pattern of coverage of the substrate by the indicator moieties, e.g., indicator cells; the amount of coverage of the substrate by the indicator moieties, e.g., indicator cells; the distribution of the indicator moieties, e.g., indicator cells, e.g., on a substrate; the amount of aggregation of the indicator moieties, e.g., indicator cells; the strength of adherence of the indicator moieties, e.g., indicator cells, to the rbcm preparation (e.g., as detected by optical trapping).

In one embodiment, the presence of the anti-RBC antigen antibody in the sample (or a positive readout) is detected by a uniform, homogenous distribution of the indicator moieties, e.g., indicator cells, on the substrate. In one embodiment, the positive readout is detected by having a coverage of the substrate by the indicator moieties, e.g., indicator cells, of at least 20%, 30%, 40%, 50%, 60%, 70%, 80%, 90%, 95%, 96%, 97%, 98%, 99% or 100% of the substrate area. An exemplary representation of a uniform distribution of the indicator moieties, e.g., indicator cells, is provided in FIG. 12B.

In another embodiment, the absence of the anti-RBC antigen antibody in the sample (or a negative read out) is detected by a non-homogeneous distribution of the indicator moieties, e.g., indicator cells, on the substrate. In one embodiment, the negative readout is detected by having a coverage of the substrate by the indicator moieties, e.g., indicator cells, of less than 99%, 95%, 90%, 85%, 80%, 75%, 70%, 60%, 50%, 40% or 30% of the substrate area (e.g., relative to what would be covered in a positive sample). In one embodiment, the negative readout is detected as a localized concentration of indicator moieties, e.g., indicator cells, e.g., as a button or a pellet. An exemplary representation of a localized (e.g., pellet) distribution of the indicator moieties, e.g., indicator cells, is provided in FIGS. 12A and 12C.

In certain embodiments, the difference in the behavior or positional distribution of the indicator moieties, e.g., indicator cells, is detected by an increased or decreased formation of an aggregate.

In one embodiment, base units of non-bound indicator moieties, e.g., indicator cells, (indicator moieties, e.g., indicator cells, not bound to the rbcm preparation) form indicator moiety, e.g., indicator cell, complexes with one another, e.g., to form aggregates of non-bound indicator moieties, e.g., indicator cells. In embodiments, said aggregate comprises at least 2, 10, 20, 50, 100, 200, 1,000, 100,000, 1,000,000, 10,000,000 or 50,000,000 base units of indicator moieties, e.g., indicator cells. In one embodiment, the aggregate is of macroscopic dimension, e.g., an aggregate having an average dimension, e.g., at its largest point, of between 140-500 um, 75 um-1 mm, 100 um and 10 mm.

In an embodiment of the method, indicator moieties, e.g., indicator cells, traverse the substrate and collides with a second (or subsequent) indicator moieties, e.g., indicator cells, e.g., a indicator moieties, e.g., indicator cells, that traverses more slowly or is bound.

In one embodiment of the method, the indicator moieties, e.g., indicator cells, e.g., indicator moiety e.g., indicator cell, complexes, e.g., an aggregate, that fails to bind to said first rbcm preparation, migrates across a substrate, e.g., into said first negative readout region of said carrier.

In other embodiments, the method further includes providing sufficient conditions, e.g., tangential velocity and sufficient time for indicator moieties, e.g., indicator cells, e.g., indicator moiety, e.g., indicator cell, complexes, e.g., an aggregate, that has not formed an immune complex to migrate across the substrate. In an embodiment, this results in uncovering substrate or reducing the amount of substrate covered by indicator moieties, e.g., indicator cells. In embodiments, the aggregate can migrate a first negative readout region.

In another embodiment, the difference in the indicator moieties, e.g., indicator cells, is detected by evaluating the strength of adherence of indicator moieties, e.g., indicator cells, to the rbcm preparation, e.g., to the substrate (e.g., as detected by optical trapping). In one embodiment, the displacement of non-bound indicator moieties, e.g., indicator cells, is evaluated by the optical trapping.

In one embodiment of the method, the presence or absence of indicator moiety, e.g., indicator cell, complexes, e.g., an aggregate, e.g., in a pre-selected location, is correlated with, respectively, the absence or presence, of said anti-RBC antigen antibody in said sample.

In another embodiment of the method, the presence, absence, or amount of detection indicator moiety, e.g., indicator cell, complexes, e.g., an aggregate, is detected in a readout region. In one embodiment, the readout region is on the rbcm preparation.

In one embodiment of the method, the detection of the presence of indicator moiety, e.g., indicator cell, complexes, e.g., an aggregate, e.g., in said readout region, is correlated with the absence or the presence of said anti-RBC antigen antibody in said sample.

The readout region can be disposed in a chamber, e.g., a well or tube.

In one embodiment of the method, said first rbcm preparation is disposed on a carrier and the presence of indicator moieties, e.g., indicator cells, that is not in indicator moiety, e.g., indicator cell, complexes, e.g., an aggregate, e.g., in a first positive readout region, of said carrier is positively correlated with the presence of an anti-first RBC antigen antibody in said sample. In another embodiment, the presence of indicator moieties, e.g., indicator cells, e.g., indicator moiety, e.g., indicator cell, complexes, e.g., an aggregate, e.g., in a first negative readout region disposed on said carrier, or on another carrier, is negatively correlated with the presence of an anti-first RBC-antigen antibody in said sample.

In certain embodiment, indicator moiety, e.g., indicator cell, complexes, e.g., an aggregate, that has not formed an immune complex migrate from said positive readout region into said negative readout region.

In other embodiments, indicator moieties, e.g., indicator cells, which have not formed an immune complex or as indicator moiety, e.g., indicator cell, complex does not migrate to negative readout region, but indicator moieties, e.g., indicator cells, which has not formed an immune complex but has formed a indicator moiety, e.g., indicator cell, complex, e.g., a macroscopic complex, migrates to a negative readout region.

In other embodiments, the first positive readout region and first negative readout regions are spatially distinct, e.g., separated, on said carrier. In one embodiment, the first readout region is disposed in a chamber, e.g., a well or tube. In another embodiment, the first negative readout region is disposed in a chamber, e.g., a well or tube. In other embodiments, the first negative readout region and a first positive readout region are disposed in a chamber, e.g., a well or tube.

In other embodiments, the method includes:

contacting said first rbcm preparation with sample from said subject under conditions sufficient for the formation of an immune complex between said first RBC antigen and anti-first RBC antigen antibody to form a first reaction mixture;

contacting said first reaction mixture with said indicator moieties, e.g., indicator cells, under conditions sufficient for the formation of an immune complex between said detection reagent and the antibody in said sample,

allowing sufficient time for indicator moieties, e.g., indicator cells, that have not formed an immune complex be detected, e.g., by detection of indicator moiety, e.g., indicator cell, complexes, e.g., an aggregate.

### Methods for Evaluating a Sample Using a Capture Agent Disposed on a Substrate or a Surface by Differential Applied Forces

In another aspect, the invention features a method of evaluating a sample for an analyte. The method can be applied to forward typing or grouping, reverse typing or grouping, antibody screening, antibody identification, extended phenotyping, or pathogen analysis. The method includes:

(a) contacting a capture agent (e.g., an antibody (e.g., an anti-RBC antibody), an antigen (e.g., an RBC antigen), an rbcm preparation, an optimized rbcm preparation) with the sample, under conditions sufficient for the formation of a complex between a capture agent, and said analyte (e.g., an antigen, an antibody or other protein having specific binding for said capture agent, e.g., in an anti-red blood cell antibody and a rbcm preparation) in said sample,

wherein, said capture agent is disposed on a substrate or a surface, e.g., a substantially planar substrate or surface, and the angle between said substrate or a surface and the direction of applied force, e.g., centrifugal, gravitational, fluid magnetic, electric or fluid, force, that causes migration of detection reagent, is non-orthogonal or other than 90 degrees (in the case of a centrifugally applied force, theta, the angle formed by the substrate or a surface and a line perpendicular to the direction of centrifugal force, is nonzero);

(b) providing a detection reagent (wherein said detection reagent can comprise a cell, e.g., a red blood cell and one or more binding agents (e.g., IgG binding agents), e.g., as an indicator moiety) under conditions sufficient for the formation of a complex, e.g., an immune complex, between said detection reagent and the analyte, e.g., anti-capture agent antibody in said sample,

(c) applying acceleration, centrifugal acceleration, at said angle such that detection reagent that does not bind to said capture agent migrates across said substrate, e.g., substantially planar substrate,

wherein the presence or absence of the analyte of interest in the sample is indicated by a value of a parameter, e.g., a measurable parameter, related to the behavior of, or positional distribution of, the detection reagent. E.g., a preselected value for a parameter related to the detection reagent, is correlated with the presence or absence of said analyte, e.g., anti-capture agent antibody, in said sample. The parameter can be, by way of example, the amount of the detection reagent (e.g., an increased or decreased presence of the detection reagent); the pattern of coverage of the substrate by the detection reagent; the amount of coverage of the substrate by the detection reagent; the distribution of the detection reagent, e.g., on a substrate; the amount of aggregation of the detection reagent; the strength of adherence of the detection reagent, to the rbcm preparation (e.g., as detected by optical trapping), as described herein,

thereby evaluating a sample for an analyte.

In one embodiment, the capture agent is a RBC antigen, e.g., at least 1, 2, 3, 4, 5, 6, 9, 10, 11, 12 or all of the RBC antigens provided in Table 1. Exemplary RBC antigens include at least 1, 2, 3, 4, 5, 6, 9, 10, 11, 12 or all of the following RBC antigens: a Rhesus antigen, e.g., one or more or all of D, C, c, E, or e; a MNS antigen, e.g., one or more or all of M, N, S, or s; a Kidd antigen, e.g., one or both of Jk^{a} or Jk^{b}; a Duffy antigen, e.g., one or both of Fy^{a} or Fy^{b}; a Kell antigen, e.g., one or both of K or k; a Lewis antigen, e.g., one or both of Le^{a} or Le^{b}; or P antigen, e.g., P1. In certain embodiments, the method includes evaluating sample from said subject for an antibody to at least the following RBC antigens: (1) D, C, E, e, c, and K; (2) D, C, E, e, c, K, Fy^{a} and Jk^{a}; and (3) D, C, E, e, c, K, Fy^{a}, Fy^{b}, Jk^{a}, Jk^{b}, S, and s.

In another embodiment, the capture agent is a pathogen antigen, e.g. a viral antigen, e.g., a viral antigen chosen from one or more of human immunodeficiency (HIV) virus, hepatitis B virus (HBV), syphilis, human T-lymphotropic virus (HTLV), hepatitis C virus (HCV), or syphilis. Exemplary pathogen antigens include an HIV 1/2 antigen, e.g., p24, p15, p17, gp36, or gp 41; a Hep B antigen, e.g., HepBsAg, or HepBcAg; or a Syphilis antigen, e.g., TmpA, p15, 17, or 47.

In other embodiments, the capture agent is an anti-RBC antigen antibody, e.g., an antibody against at least 1, 2, 3, 4, 5, 6, 9, 10, 11, 12 or all of the RBC antigens provided in Table 1, e.g., an RBC antigen described herein. Exemplary anti-RBC antigen antibodies include antibodies against at least 1, 2, 3, 4, 5, 6, 9, 10, 11, 12 or all of the following RBC antigens: a Rhesus antigen, e.g., one or more or all of D, C, c, E, or e; a MNS antigen, e.g., one or more or all of M, N, S, or s; a Kidd antigen, e.g., one or both of Jk^{a} or Jk^{b}; a Duffy antigen, e.g., one or both of Fy^{a} or Fy^{b}; a Kell antigen, e.g., one or both of K or k; a Lewis antigen, e.g., one or both of Le^{a} or Le^{b}; or P antigen, e.g., P1. In certain embodiments, the method includes evaluating sample from said subject for an antibody to at least the following RBC antigens: (1) D, C, E, e, c, and K; (2) D, C, E, e, c, K, Fy^{a} and Jk^{a}; and (3) D, C, E, e, c, K, Fy^{a}, Fy^{b}, Jk^{a}, Jk^{b}, S, and s.

In yet other embodiments, the capture agent is an anti-pathogen antigen antibody, e.g., an antibody against a viral antigen, e.g., a viral antigen chosen from one or more of human immunodeficiency (HIV) virus, hepatitis B virus (HBV), syphilis, human T-lymphotropic virus (HTLV), hepatitis C virus (HCV), or syphilis, e.g., a viral antigen as described herein.

In yet other embodiments, the capture agent is a CMV, WNV, HTLV-1 and -2, or platelet antigen, or an antibody against same.

In certain embodiments, two different forces are applied, a first force to provide force normal to the substrate or a surface and a second force to provide force tangential to said substrate or a surface. In one embodiment, the first force, e.g., a magnetic force, is applied to produce force normal to said substrate or a surface on a detection reagent complex or aggregate, and a second force, e.g., fluid force, is applied to produce force tangential to said substrate or a surface on a detection reagent complex or aggregate.

In another aspect, the invention features a method of evaluating a sample, e.g., a plasma sample, from a subject, for an anti-RBC antigen antibody. The method can be applied to reverse typing or grouping, antibody screening, or antibody identification. The method includes:

(a) contacting a first red blood cell membrane (rbcm) preparation with sample from said subject, under conditions sufficient for the formation of an immune complex between a first RBC antigen and the anti-first RBC antigen antibody in said sample,

wherein, said first rbcm preparation is disposed on a substrate, e.g., a substantially planar substrate, and the angle between said substrate, e.g., substantially planar substrate, and the direction of applied force, e.g., centrifugal, gravitational, magnetic, electric or fluid, force, that causes migration of detection reagent, is non-orthogonal or other than 90 degrees (in other words, theta, the angle formed by the substrate and a line perpendicular to the direction of centrifugal force, is nonzero);

(b) providing a detection reagent under conditions sufficient for the formation of a complex, e.g., an immune complex, between said detection reagent and an anti-RBC antigen antibody in said sample,

(c) applying force, e.g., centrifugal force, at said angle such that detection reagent that does not bind to said first rbcm preparation migrates across said substrate,

wherein the presence or absence of the analyte of interest in the sample is indicated by a value of a parameter, e.g., a measurable parameter, related to the behavior of, or positional distribution of, the detection reagent. E.g., a preselected value for a parameter related to the detection reagent, is correlated with the presence or absence of said anti-first RBC antigen antibody, in said sample. The parameter can be, by way of example, the amount of the detection reagent (e.g., an increased or decreased presence of the detection reagent); the pattern of coverage of the substrate by the detection reagent; the amount of coverage of the substrate by the detection reagent; the distribution of the detection reagent, e.g., on a substrate; the amount of aggregation of the detection reagent; the strength of adherence of the detection reagent, to the rbcm preparation (e.g., as detected by optical trapping), as described herein,

thereby evaluating a sample for an anti-RBC antigen antibody.

In an embodiment, force is applied such the ratio of normal force/tangential force will decrease with time, e.g., decrease in a continuous or non-continuous (e.g., in discrete steps), e.g., by increase of the tangential force over time.

In another aspect, the invention features a method of evaluating a sample, e.g., a plasma sample, from a subject, for an anti-RBC antigen IgG antibody comprising:

(a) contacting a first red blood cell membrane (rbcm) preparation with sample from said subject, under conditions sufficient for the formation of an immune complex between a first RBC antigen and anti-first RBC IgG antigen antibody in said sample, wherein,
(i) said first rbcm preparation is disposed on a substrate, e.g., a substantially planar substrate, and the angle between said substrate and the direction of applied force, e.g., centrifugal, gravitational, magnetic, electric or fluid, force, that causes migration of detection reagent, is non-orthogonal or other than 90 degrees (theta, the angle formed by the substrate and a line perpendicular to the direction of centrifugal force, is nonzero);
(ii) said substrate having said first rbcm preparation, bound thereto, has one of the following properties:
(A) if red blood cells are dispersed on said substrate having said first rbcm preparation bound thereto, less than 10, 5, or 1% of the dispersed red blood cells are non-specifically bound, e.g., as determined by optical trap measurement;
(B) if red blood cells are dispersed on said substrate having said rbcm preparation bound thereto, the non-specific binding of dispersed red blood cells to said substrate having said first rbcm preparation bound thereto, is less than 50, 40, 30, 20, 10, 1.0, 0.1, or 0.01% of the non-specific binding of red blood cells to a reference substrate, e.g., a substantially planar substrate having a rbcm preparation, bound thereto, prepared in a similar manner except that the red blood cells which are lysed to form a rbcm preparation are deposited on the substrate by gravitational settling as opposed to centrifugation; and
(iii) optionally, said rbcm preparation is a mo-rbcm preparation, e.g., it was contacted with an agent that cleaves IgG molecules, e.g., an enzyme, e.g., IdeS (immunoglobulin G-degrading enzyme of S. pyrogenes), e.g., FabRICATOR®;

(b) providing a detection reagent that specifically binds IgG antibodies under conditions sufficient for:

(i) the formation of a complex, e.g., an immune complex, between said detection reagent and an anti-RBC antigen IgG antibody in said sample; and

(ii) the detection reagent complexation of base units of detection reagent with one another; and

(c) applying centrifugal acceleration at said angle such that detection reagent that does not bind to said first rbcm preparation complexes with itself and migrates across said substrate, wherein, the position and degree of detection reagent complex or aggregate formation of detection reagent is correlated with the presence or absence of said anti-first RBC antigen antibody in said sample, thereby evaluating a sample for an anti-RBC antigen antibody.

In one embodiment, the substrate is configured such that said angle of said substrate can be altered, e.g., to provide a first angle for a first phase of centrifugation, and a second angle for a second phase of centrifugation.

In yet another aspect, the invention features a method of evaluating a sample for an analyte comprising:

(a) contacting a capture agent (e.g., an antibody, an antigen, e.g., a rbcm preparation, e.g., an mimic optimized-rbcm preparation) with sample, under conditions sufficient for the formation of a complex between a capture agent, and said analyte (e.g., an antigen, an antibody or other protein having specific binding for said capture agent, e.g., in an anti-red blood cell antibody and a rbcm preparation) in said sample, wherein, said capture agent is disposed on a substrate or surface, e.g., a substantially planar substrate or surface;

(b) providing a detection reagent comprising a deformable entity, e.g., a cell, e.g., a red blood cell, or another entity having similar deformability or size, and a binding agent, under conditions sufficient for the formation of a complex, e.g., an immune complex, between said detection reagent and analyte, e.g., anti-capture agent antibody in said sample,

(c) applying acceleration, centrifugal acceleration, at said angle such that detection reagent that does not bind to said capture agent migrates across said substrate or surface, e.g., a substantially planar substrate or surface,

wherein the presence or absence of the analyte of interest in the sample is indicated by a value of a parameter, e.g., a measurable parameter, related to the behavior of, or positional distribution of, the detection reagent. E.g., a preselected value for a parameter related to the detection reagent, is correlated with the presence or absence of said analyte, e.g., anti-capture agent antibody, in said sample. The parameter can be, by way of example, the amount of the detection reagent (e.g., an increased or decreased presence of the detection reagent); the pattern of coverage of the substrate by the detection reagent; the amount of coverage of the substrate by the detection reagent; the distribution of the detection reagent, e.g., on a substrate; the amount of aggregation of the detection reagent; the strength of adherence of the detection reagent, to the rbcm preparation (e.g., as detected by optical trapping), as described herein,

In one embodiment, base units of non-bound detection reagent (e.g., detection reagent not bound to a capture agent) form detection reagent complexes with one another, e.g., to form aggregates of non-bound detection reagent.

In yet other embodiment, non-bound detection reagent, e.g., detection reagent complexes, e.g., an aggregate, is separated from detection reagent bound to a capture agent.

In one embodiment, the deformable entity, e.g., a red blood cell, allows the cell to explore more surface area (e.g., have more surface to surface contact) as it transits the substantially planar substrate, e.g., as it transits said substantially planar substrate in response to the applied tangential force.

In one embodiment, the deformability of the deformable entity, e.g., a red blood cell, promotes detection reagent complexation in a negative sample and promotes migration across the substantially planar substrate.

### Method of Providing a Substrate

In another aspect, the invention features a method of providing a substrate having red blood cells, or a red blood cell membrane preparation, bound thereto comprising:

providing a substrate capable of binding red blood cells;

contacting said substrate with a solution of red blood cells to form a solution-contacted-substrate;

centrifuging said solution-contacted-substrate for a time sufficient to cause red blood cells in said solution to settle onto said substrate;

optionally, washing said substrate to remove unbound red blood cells;

optionally, lysing red blood cells bound to said substrate to provide a rbcm preparation bound to said substrate;

thereby providing a substrate having red blood cells, or a rbcm preparation, bound thereto,

wherein, optionally, said substrate having red blood cells, or a rbcm preparation, bound thereto, has one of the following properties:
said centrifugation is sufficient in force and duration such that, if red blood cells are dispersed on the substrate having red blood cells, or rbcm preparation, bound thereto, less than 10, 5, or 1% of the dispersed red blood cells are non-specifically bound, e.g., as determined by optical trap measurement;
said centrifugation is sufficient in force and duration such that if red blood cells are dispersed on the substrate having red blood cells, or rbcm preparation, bound thereto, the non-specific binding of red blood cells to said substrate having red blood cells, or rbcm preparation, bound thereto, is less than 50, 40, 30, 20, 10, 1.0, 0.1, or 0.01% of the non-specific binding of red blood cells to a reference substrate, e.g., a substrate having red blood cells, or rbcm preparation, bound thereto, prepared in a similar manner except that the red blood cells are deposited on the substrate by gravitational settling as opposed to centrifugation.

In certain embodiments, the method further includes lysing red blood cells bound to said substrate, thereby providing a substrate having a rbcm preparation bound thereto.

In one embodiment, the solution-contacted-substrate is centrifuged at 400 g for 5 minutes at 20 degree C., in saline, or under conditions sufficient to give a similar level of non-specific binding.

The substrate can include glass or plastic. In one embodiment, the substrate is derivatized with aminopropyltriethoxysilane, poly-1-lysine, or Alcian Blue.

In certain embodiments, the substrate is a multi-well plate, e.g., a 96 well plate, e.g., a polystyrene 96 well plate.

In other embodiments, the red blood cells, or rbcms, are present on the substrate at between 14000-24000, 24000-34000 and 34000-40000, cells/mm², e.g., at 26,000 cells/mm².

In yet other embodiments, the centrifugation is sufficient in force and duration such that if red blood cells are dispersed on the substrate having red blood cells, or rbcm preparation, bound thereto, less than 10, 5, or 1% of the dispersed red blood cells are non-specifically bound, e.g., as determined by optical trap measurement.

In one embodiment, the centrifugation is sufficient in force and duration such that if red blood cells are dispersed on the substrate having red blood cells, or rbcm preparation, bound thereto, the non-specific binding of red blood cells to said substrate having red blood cells, or rbcm preparation, bound thereto, is less than 50, 40, 30, 20, 10, 1.0, 0.1, or 0.01% of the non-specific binding of red blood cells to a reference substrate, e.g., a substrate having red blood cells, or rbcm preparation, bound thereto, prepared in a similar manner except that the red blood cells are deposited on the substrate by gravitational settling as opposed to centrifugation.

In one embodiment, the rbcm preparation is contacted with an agent that cleaves IgG molecules, e.g., an enzyme, e.g., IdeS (immunoglobulin G-degrading enzyme of S. pyrogenes), e.g., FabRICATOR®, thereby producing a mimic optimized-rbcm preparation.

### Substrates

In another aspect, the invention features a substrate having red blood cells, or a rbcm preparation, e.g., a mimic optimized-rbcm preparation, bound thereto, wherein if red blood cells are dispersed on the substrate having red blood cells, or rbcm preparation, bound thereto, less than 10, 5, or 1% of the dispersed red blood cells are non-specifically bound, e.g., as determined by optical trap measurement.

In yet another aspect, the invention features substrate having red blood cells, or a rbcm preparation, e.g., a mo-rbcm preparation, bound thereto, wherein if red blood cells are dispersed on the substrate having red blood cells, or rbcm preparation, bound thereto, the non-specific binding of dispersed red blood cells to said substrate having red blood cells, or a rbcm preparation, bound thereto, is less than 50, 40, 30, 20, 10, 1.0, 0.1, or 0.01% of the non-specific binding of dispersed red blood cells to a reference substrate, e.g., a substrate having red blood cells, or rbcm preparation, bound thereto, prepared in a similar manner except that the red blood cells are deposited on the substrate by gravitational settling as opposed to centrifugation.

In another aspect, the invention features a substrate having red blood cells, or a rbcm preparation, e.g., a mo-rbcm preparation, bound thereto, made by the method of claim 96, wherein if red blood cells are dispersed on the substrate having red blood cells, or rbcm preparation, bound thereto, less than 10, 5, or 1% of the dispersed red blood cells are non-specifically bound, e.g., as determined by optical trap measurement.

In one aspect, the invention features a substrate having red blood cells, or a rbcm preparation, e.g., a mo-rbcm preparation, bound thereto, made by the method of claim 96, wherein if red blood cells are dispersed on the substrate having red blood cells, or rbcm preparation, bound thereto, the non-specific binding of dispersed red blood cells to said substrate having red blood cells, or a rbcm preparation, bound thereto, is less than 50, 40, 30, 20, 10, 1.0, 0.1, or 0.01% of the non-specific binding of dispersed red blood cells to a reference substrate, e.g., a substrate having red blood cells, or rbcm preparation, bound thereto, prepared in a similar manner except that the red blood cells are deposited on the substrate by gravitational settling as opposed to centrifugation.

### Devices

In another aspect, the invention features a device for evaluating a sample, e.g., a plasma sample, from a subject, for an anti-RBC antigen antibody, comprising:

a channel comprising
a) a substrate having red blood cells, or a rbcm preparation, e.g., a mo-rbcm preparation, bound thereto, wherein
   if red blood cells are dispersed on the substrate having red blood cells, or rbcm preparation, bound thereto, less than 10, 5, or 1% of the dispersed red blood cells are non-specifically bound, e.g., as determined by optical trap measurement; or
   if red blood cells are dispersed on the substrate having red blood cells, or rbcm preparation, bound thereto, the non-specific binding of dispersed red blood cells to said substrate having red blood cells, or a rbcm preparation, bound thereto, is less than 50, 40, 30, 20, 10, 1.0, 0.1, or 0.01% of the non-specific binding of dispersed red blood cells to a reference substrate, e.g., a substrate having red blood cells, or rbcm preparation, bound thereto, prepared in a similar manner except that the red blood cells are deposited on the substrate by gravitational settling as opposed to centrifugation;

wherein the device is configured such that, upon application of a force, e.g., centrifugal, gravitational, fluid magnetic, electric or fluid, force, detection reagent that has not formed an immune complex can: form a detection reagent complex, e.g., to form an aggregate; migrate into a negative readout region; or, both from a detection reagent complex, e.g., form an aggregate and migrate into a negative readout region.

In another aspect, the invention features a device for evaluating a sample, e.g., a plasma sample, from a subject, for an anti-RBC antigen antibody, comprising:

a channel comprising
red blood cells, or a first rbcm preparation e.g., a mo-rbcm preparation, disposed on a substantially planar substrate, and the angle between said substantially planar substrate and the direction of applied force, e.g., centrifugal, gravitational, magnetic, electric or fluid, force, that causes migration of detection reagent, is other than 90 degrees;

wherein the device is configured such that, upon application of a force, e.g., centrifugal, gravitational, magnetic, electric or fluid, force, detection reagent that has not formed an immune complex can: form a detection reagent complex, e.g., form an aggregate; migrate into a negative readout region; or, both form a detection reagent complex, e.g., form an aggregate, and migrate into a negative readout region.

Additional features of the devices of the invention include one or more of the following:

In one embodiment, the detection reagent complexation, e.g., aggregation, in the presence or absence of said detection reagent in a negative readout region; or detection reagent complexed, e.g., aggregated, detection reagent in said negative readout region, is correlated with the presence or absence an anti-blood-type-antigen antibody in said sample.

In one embodiment, the device includes a plurality of said channels, each having a different rbcm preparation. In yet other embodiment, each channel in said plurality is fluidically isolated from the other channels of the plurality.

In one embodiment, the device has a first channel comprising a first rbcm having a first RBC antigen (e.g., an RBC antigen as described herein) and a second channel comprising a second rbcm having a second RBC antigen. For example, the first antigen can be antigen A and said second antigen can be antigen B.

In other embodiment, the rbcm preparation in the device is contacted with an agent that cleaves IgG molecules, e.g., an enzyme, e.g., IdeS (immunoglobulin G-degrading enzyme of S. pyrogenes), e.g., FabRICATOR®.

In other embodiments, the device is configured such that said angle of said substantially planar substrate can be altered, e.g., to provide a first angle for a first phase of centrifugation, and a second angle for a second phase of centrifugation.

In another aspect, the invention features a device for evaluating a sample, e.g., a plasma sample, from a subject, for one or a plurality of different anti-RBC antigen antibodies comprising:

a plurality of channels, e.g., at least 3, 6, 12, or 24 channels, each channel comprising
a) a capture region for receiving RBC or a rbcm preparation, e.g., a mo-rbcm preparation, disposed on a substantially planar substrate, and the angle between said substantially planar substrate and the direction of applied force, e.g., centrifugal, gravitational, magnetic, electric or fluid, force, that causes migration of detection reagent, is other than 90 degrees;

wherein the device is configured such that, upon application of a force, e.g., centrifugal or gravitational force, detection reagent that has not formed an immune complex can: form detection reagent complex, e.g., form an aggregate; migrate into a negative readout region; or, both from a detection reagent complex, e.g., form an aggregate, and migrate into a negative readout region.

Methods of making the aforesaid devices are also encompassed by the present invention.

### Kits

In yet another aspect, the invention features a kit that includes the rbcm preparation as described herein. In certain embodiments, the kit further includes one or some or all of:

(a) detection reagent having a binding moiety as described herein;

(b) a detection reagent complexing agent that promotes detection reagent complexation between base units of detection reagent;

(c) a positive control sample, e.g., a sample having an antibody to a pre-selected blood type antigen;

(d) a negative control sample, e.g., a sample lacking an antibody to a pre-selected blood type antigen;

(e) a first rbcm preparation, e.g., made by a method described herein;

(f) a carrier on which said rbcm is or can be disposed; and

(g) an agent that cleaves IgG, e.g., IdeS (immunoglobulin G-degrading enzyme of S. pyrogenes), e.g., FabRICATOR®, for preparing a mo-rbcm preparation.

In certain embodiments, a panel of rbcm preparations described herein is disposed on capture regions of said device.

Thus, in an embodiment the substrate is a substantially planar substrate, and the angle between said substantially planar substrate and the direction of applied force, e.g., centrifugal, gravitational, fluid magnetic, electric or fluid, force, that causes migration of forward detection reagent, is non-orthogonal or other than 90 degrees (theta, the angle formed by the substantially planar substrate and a line perpendicular to the direction of centrifugal force, is nonzero.

Any of the features and embodiments described herein, e.g., methods (e.g., forward typing, reverse typing, antibody screening, antibody isolation, Ig detection), IgG-specific binding moieties, optimized substrates and substrate angles, and rbcm preparations (e.g., density optimized rbcm preparations and mimic-optimized preparations) described herein, can be combined in any order with the described methods, and/or implemented on devices and kits described herein. In one embodiment, a forward typing, antibody screening and/or reverse typing method or assay is combined, e.g., on the same carrier, and/or processed simultaneously.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

All publications, patent applications, patents, and other references mentioned herein are incorporated by reference in their entirety.

Other features, objects, and advantages of the invention will be apparent from the description and drawings, and from the claims.

### DESCRIPTION OF THE FIGURES

FIGS. 1A-ID illustrate side views of an embodiment of forward typing well configurations and testing, and a top view of the readout regions.

FIGS. 1E-1G show a representative panel of photographs depicting the positive and negative readouts of the forward typing assays.

FIGS. 2A-2E illustrate side views of an embodiment of reverse grouping well configurations and testing, and a top view of the readout regions.

FIGS. 3A-3D illustrate side views of an embodiment of extended phenotyping well configurations and testing, and a top view of the readout regions.

FIGS. 4A-4D illustrate side views of the stepwise changes in the well configurations according to one embodiment of the antibody screening assays.

FIGS. 5A-5D illustrate side views of the stepwise changes in the chamber configurations according to one embodiment of the antibody screening assays after performing a centrifugation step, and a top view of the readout regions.

FIGS. 6A-6C illustrate side views of the stepwise changes in the chamber configurations according to the antibody screening assays after performing optical trapping detection.

FIGS. 7A-7B depict a schematic representation of one embodiment of the antibody screening assays described herein, and a representative graph showing the percentage of red blood cells detected as bound as a function of secondary incubation time.

FIG. 7C depicts a representative graph showing the percentage of red blood cells detected as bound as a function of secondary incubation time using the antibody screening assays described herein.

FIGS. 8A-8D provide a stepwise representation of the components of the antibody screening assays described in FIG. 7A.

FIG. 9 provides a representative graph showing a comparison of the nonspecific binding to a red blood cell membrane preparation using a panel of anti-IgG antibodies. The percentage of red blood cells detected as bound as a function of secondary incubation time is depicted.

FIG. 10 provides a representative graph depicting binding of MS-278 monoclonal anti-IgG to two different red blood cell membrane preparations, one positive for the D RBC antigen (#2 Cells D+) and one negative for the D RBC antigen (#3 Cells D−), in the presence of anti-D, as revealed by indicator cells (IgG-coated red cells).

FIG. 11 provides a representative graph depicting binding of rabbit polyclonal anti-IgG (Alba #Z356) to non-treated and enzyme treated red blood cell membrane preparations.

FIGS. 12A-12C show a representative panel of photographs depicting the positive and negative readouts of the ABO reverse grouping assays.

FIG. 13A is a schematic top plane view of a centrifuge operating in a clockwise direction.

FIGS. 13B-13C are schematic views illustrating forces as applied to objects on an incline plane disposed in an operating centrifuge.

FIG. 14 illustrates schematic and/or perspective view representations of exemplary chamber configurations.

FIGS. 15A-15C are schematic views of a reverse grouping configuration and testing.

FIGS. 16A-16B are representative photographs of positive and negative readouts for antibody screening assays detected using a low concentration of indicator cells.

FIGS. 16C-16D are representative photographs of positive and negative readouts for antibody screening assays detected using a high concentration of indicator cells.

FIGS. 17A-17F illustrate side and perspective views of a number of exemplary substrate configurations.

FIG. 18 is a representative photograph of positive and negative readouts detected using antigen typing assays.

### DETAILED DESCRIPTION

The present invention provides, at least in part, methods and devices for evaluating a sample, e.g., a plasma sample, from a subject, for detecting a target molecule, e.g., an antibody (e.g., IgM-, IgG, IgE, an anti-red blood cell (RBC)-antigen antibody, and an anti-pathogenic antibody); an RBC antigen, a viral or pathogenic antigen). In one embodiment, the antigen is a RBC antigen, e.g., a Rhesus antigen, an MNS antigen, a Kidd antigen, a Duffy antigen, a Kell antigen, a Lewis antigen, or one or more antigens according to Table 1). The present invention can be applied to screening and blood typing, including forward typing, reverse grouping, antibody screening (IgM and IgG class antibodies), antibody identification, minor antigen typing, and extended phenotyping. In other embodiments consistent with the present invention, methods and devices disclosed herein are suitable for infectious disease screening (e.g., human immunodeficiency (HIV) virus, hepatitis B virus (HBV), syphilis, human T-lymphotropic virus (HTLV), hepatitis C virus (HCV), syphilis, among others), by testing for antibodies to these infectious agents or in some cases testing for the agents themselves. In yet other embodiments, the invention can be applied to allergy testing (e.g., IgE antibody testing).

In one embodiment, Applicants have discovered optimized antibody screening methods and devices that significantly reduce the level of background, non-specific binding to a surface (e.g., a test surface bound with a red blood cell (RBC) membrane preparation that includes an RBC antigen described herein), thus allowing for more efficient detection and reduced test time.

In another embodiment, Applicants have discovered that ruptured human red blood cells (e.g., a human red blood cell membrane preparation described herein) lead to non-specific binding of several commercially available anti-IgG antibodies. Without wishing to be bound by theory, it is believed that rupturing red blood cells to produce the rbc membrane preparation unmasks an IgG-mimic that is recognized by such antibodies. At least two different embodiments for decreasing the non-specific binding caused by the ruptured red blood cell membrane preparations are disclosed in the present application.

In one embodiment, IgG binding moieties that bind selectively and specifically to the plasma IgG present, relative to the binding to the rbcm preparation, are disclosed. In one embodiment, the IgG binding moieties' non-specific binding to the rbcm preparation is decreased by at least 10%, 20%, 30% or more (e.g., as determined by optical trapping). In another embodiment, the IgG-specific binding moiety includes an antibody molecule that binds to an IgG, e.g., an antibody molecule that binds to a constant region (e.g., a heavy chain Fc region or a light chain constant region) of the IgG, or a heavy or light chain variable domain of the IgG. In certain embodiments, the IgG-specific binding moiety includes an antibody molecule that has one or more of the properties of monoclonal antibody MS-278 (e.g., the IgG-specific binding moiety comprises a monoclonal antibody MS-278, or an antigen binding fragment thereof). In other embodiments, the IgG-specific binding moiety includes an antibody molecule that binds to a light chain constant region.

Alternatively, or in combination with, the methods described herein, non-specific binding caused by the lysed red blood cell membrane preparation can be reduced by an agent that disrupts the IgG mimic (e.g., an enzyme that cleaves IgG) present on the rbcm preparations, thereby producing a mimic-optimized rbcm preparation. In one embodiment, the agent is an enzyme, e.g., a cysteine proteinase, with specificity for immunoglobulin G. In one embodiment, the enzyme is an immunoglobulin-degrading enzyme of S. pyogenes (e.g., IdeS). Mimic-optimized rbcm preparation are also encompassed by the present invention.

In another aspect, the invention provides methods and devices for target capturing that include a surface or substrate, e.g., a substantially planar surface or substrate, optionally having an optimized angle, for capture. Alternative solid phase geometries for capture are disclosed.

In another aspect, optimized methods for cell deposition are disclosed.

In another aspect, the invention provides methods and devices for determining the presence or absence of red blood cell minor group antigens using surfaces presenting antibodies to each said minor red cell antigen. Applicants have developed a simplified system including the preparation of the antibodies, preparation of suitable surfaces presenting said antibodies, and the parameters which enable such a test to be performed successfully. These devices and methods are suitable for minor antigen typing, as well as red cell phenotyping.

The invention also discloses devices, kits, assays that rely on one of more of the embodiments disclosed herein.

### DEFINITIONS

Certain terms are first defined.

“Antibody identification,” as used herein, refers to a series of tests used to determine the specificity of the one or more antibodies present in a plasma sample which give rise to a positive antibody screening test result. For example, if a positive blood group antibody screen is obtained, blood group antibody identification will comprise a series of tests of the reactivity of the plasma sample with substrates or reagents to determine the particular blood group antigen specificity(ies) of the antibody(s).

“Antibody screening,” as used herein, refers to the detection of non-native (elicited) antibodies specific to an antigen. These antibodies can be IgG and IgM antibodies. In one embodiment, the antigen is a red blood cell (RBC) antigen. Red blood cell antigens are antigens found on the surface of the red blood cells, and include, but are not limited to, the roughly 600 blood group antigens known to date. In certain embodiments, the blood group antigens include the A and B antigens, as well as antigens of the Rh system, e.g., D antigens. Other exemplary red blood cell antigens include an MNS antigen, a Kidd antigen, a Duffy antigen, a Kell antigen, a Lewis antigen and a P antigen (e.g., an antigen listed in Table 1).

In other embodiments, the antigen detected by antibody screening is a viral antigen (e.g., human immunodeficiency (HIV) virus, hepatitis B virus (HBV), syphilis, human T-lymphotropic virus (HTLV), hepatitis C virus (HCV), among others).

“Antibody testing,” as used herein, can refer to testing for the presence of one or more plasma antibodies, e.g., anti-IgG antibodies.

“Blood group,” or “blood type,” as used herein refers to any of the immunologically distinct, genetically determined classes of human blood that are based on the presence or absence of certain antigens. Blood groups are typically clinically identified by characteristic agglutination reactions. Blood group antigens which are typically associated with the ABO blood group system, and includes the A, B, AB, and O blood groups.

“Blood typing,” as used herein, refers to ABO and D antigens. Blood types are typically classified as ABO Rh “blood type” commonly listed on the donor cards for blood donors (e.g., A Rh Pos, A Rh Neg, B Rh Pos, B Rh Neg, O Rh Pos, O Rh Neg, AB Rh Pos, AB Rh Neg).

“Detection reagent complex” or “detection reagent aggregate,” as used herein, refers to a plurality of base units of detection reagent held together by an interaction, e.g., an interaction mediated by non-covalent bonds. It refers to an interaction between base units of detection reagents and not to an interaction between detection reagent and a target analyte, e.g., antigen. A detection reagent complex or a detection reagent aggregate migrates as a single entity, e.g., across the surface of a rbcm preparation on a substantially planar substrate. In an embodiment a detection reagent complex is easier to detect than non complexed detection reagent, e.g., because it presents an aggregate that can be optically detected, e.g., by spectroscopy or by visual inspection.

Typically, an aggregate is of macroscopic dimension, e.g., comprising greater than 100 base units of detection reagent. Typically, an aggregate is of detectably distinct character, comprising enough distinct units to have qualitatively distinct behavior and appearance from non-aggregated units. Typically an aggregate is of distinct character under external forcing, e.g., moving at a much larger speed under centrifugation than uncomplexed base units of detection reagent. An aggregate can comprise at least 2, 10, 20, 50, 100, 200, 1,000, 10,000, 1,000,000, 10,000,000, or 50,000,000 base units of detection reagent. An aggregate can have macroscopic dimension, e.g., an aggregate having a dimension, e.g., along its largest dimension, of between 140-500 um, 75 um-1 mm, 50 um and 10 mm.

In one embodiment, the detection reagent comprises a deformable component, e.g., a cell (e.g., a red blood cell), or an entity which is similarly deformable.

“Detection reagent,” as used herein, has a binding moiety capable of binding to an analyte, e.g., binding to an antibody, e.g, an IgG-specific binding moiety. A base unit (or unit) of detection reagent typically comprises an indicator moiety, e.g., a red blood cell, and one or more binding agents, e.g., IgG binding agents, e.g., IgG-specific binding agents. In embodiments base units of detection reagent are capable of complexing to form aggregates. A detection reagent can include a moiety that promotes aggregation between detection reagent units, e.g., and anti-D antibody.

“Extended phenotyping”, as used herein, refers to testing for the presence or absence of each of a collection of red cell minor blood group antigens on the surface of a sample of red blood cells. For example, an extended phenotype could test for each of D, C, c, E, e, and K. As another example, and extended phenotype could test for each of D, C, c, E, e, K, Jk^{a}, Jk^{b}, Fy^{a}, Fy^{b}, S, and s. As another example, an extended phenotype could test for each of D, C, c, E, e, K, k, Jk^{a}, Jk^{b}, Fy^{a}, Fy^{b}, M, N, S, s, Le^{a}, Le^{b}, and P1, which may be referred to specifically as a “complete extended phenotype” or “full extended phenotype”.

“Forward typing,” as used herein, refers to determination of the A/B/O/D type by detecting the presence or absence of A, B, and D antigens on red blood cells.

“IgG mimic,” as used herein, refers to an epitope on rbcm preparations that is bound by some anti-IgG antibodies, e.g., Alba Z356. In certain embodiments, the IgG mimic can be partially inactivated by treatment with a proteolytic enzyme, e.g., IdeS (immunoglobulin G-degrading enzyme of S. pyrogenes), e.g., FabRICATOR®.

“IgG-specific binding moiety,” is a moiety that shows sufficient specificity for IgG, as opposed to an rbcm preparation (e.g., as mediated by an IgG mimic on rbcm preparations) to allow for use in the method described herein, e.g., it shows a specificity described herein.

“Mimic-optimized (mo) rbcm preparation,” as used herein, is a rbcm preparation that has been exposed to a treatment that partly or entirely neutralizes, the IgG mimic. In embodiments, the rbcm preparation is contacted with an agent that binds to or cleaves IgG, antibodies, e.g., in the Fc region. The treatment inactivates, e.g., by cleavage or binding or masking, epitopes on the rbcm preparations that mimic IgG and are associated with binding of anti-IgG antibodies to rbcm preparations. In an embodiment a mimic optimized-rbcm preparation is produced by contacting the rbcm preparation (or cells from which it is made) with a proteolytic enzyme, e.g., IdeS (immunoglobulin G-degrading enzyme of S. pyrogenes), e.g., FabRICATOR®. In an embodiment the agent is an anti-IgG antibody that is itself not bound by an IgG-specific binding agent. E.g., it can be an anti-IgG fragment or an anti-IgG antibody of other than G isotype.

“Minor antigen typing,” as used herein, refers to testing for the presence or absence of one or several specific red cell minor blood group antigens on the surface of a sample of red blood cells. For example, a minor antigen type test may test for the E antigen. As another example, one may perform minor antigen typing for both the K antigen and Jk^{a} antigen, wherein one performs a minor antigen test for each of K and Jk^{a}.

“Negative readout region,” as used herein, is a region in which a signal can indicate the absence of an analyte.

“Positive readout region,” as used herein, is a region in which a signal can indicate the presence of an analyte.

“Readout region,” as used herein, is a region, e.g., a pre-selected region, from which a signal, e.g., a signal corresponding to the presence or absence of an analyte, is collected.

“Red blood cell membrane preparation” (a rbcm preparation) as used herein, refers to lysed red blood cells. Typically, the lysed red blood cell membranes are bound to a substrate, e.g., a substantially planar substrate with sufficient affinity to allow the manipulations in the methods described herein.

“Reverse grouping,” as used herein, refers to the determination of A/B/O group by detecting the presence or absence of native antibodies, typically IgM antibodies, specific to A and B antigens (ie. anti-A, anti-B, anti-AB) in blood plasma or serum.

“Substantially planar substrate,” as used herein, means a substrate or a region of a substrate, which has one or more of the following properties:

(1) it is sufficiently planar that the desired ratio of normal force and tangent force can be maintained precisely or approximately throughout the substantially planar substrate;

(2) the surface vector S (which is normal to the surface of the substantially planar substrate region) is constant or does not does not vary in angle, relative to its average, by more than 2, 5, or 10 degrees across the substantially planar substrate:

(3) the angle between the surface vector S (which is normal to the surface of the substantially planar substrate region) and a reference vector R, e.g., the symmetry axis of a cone, is constant, or varies by no more than 2, 5, or 10 degrees, across the substantially planar substrate substrate (thus, the surface of a perfect cone is a substantially planar substrate, as is a region of a paraboloid in the vicinity of its symmetry axis); or

(4) the ratio of the normal force to the tangent force does not exceed 110%, 130%, or 200%, or fall below 90%, 70% or 50%, of its average value within the substantially planar substrate.

When disposed in a well, tube or other enclosure, the substantially planar substrate need not occupy the entire bottom of the enclosure. The substantially planar substrate may be continuous with other substrate regions that are not substantially planar. In an embodiment, the substantially planar substrate has a surface area of 20-200, 4-40, 0.4-10, or 0.2-10 mm². In an embodiment the substantially planar substrate is of sufficient area that it allows development of a substantial difference in migration between a detection reagent aggregate, e.g., one that includes at least 50, 100, or 200 base units of detection reagent and detection reagent base units that are not detection reagent complexed.

A well, tube, or other enclosure, for use in a method of device described herein can comprise one or a plurality of substantially planar substrates. Substantially planar substrates can be disposed on the same, or different substrates. In an embodiment having a plurality of substantially planar substrates in one well tube or other enclosure, the surface area of the plurality is 20-200, 4-40, 0.4-10, or 0.2-10 mm².

Substantially planar does not require a smooth surface. In embodiments, substantially planar substrate can have surface texturing, e.g., it can be grooved or have a roughened or dimpled surface. In an embodiment the average displacement between the lowest and highest points of the features is less than 10 microns, 1-100 microns, or 10-50 microns. For the preceding determination of the surface vector in cases where the surface has structure or texture on length scales smaller than 5, 10, 25, or 50 microns, the surface vector is taken to be the vector normal to the “average surface” at that point, where the average surface is calculated by fitting the neighborhood of size 5, 10, 25, or 50 microns to a plane. In one embodiment, the surface of the substantially planar substrate resides substantially in a plane.

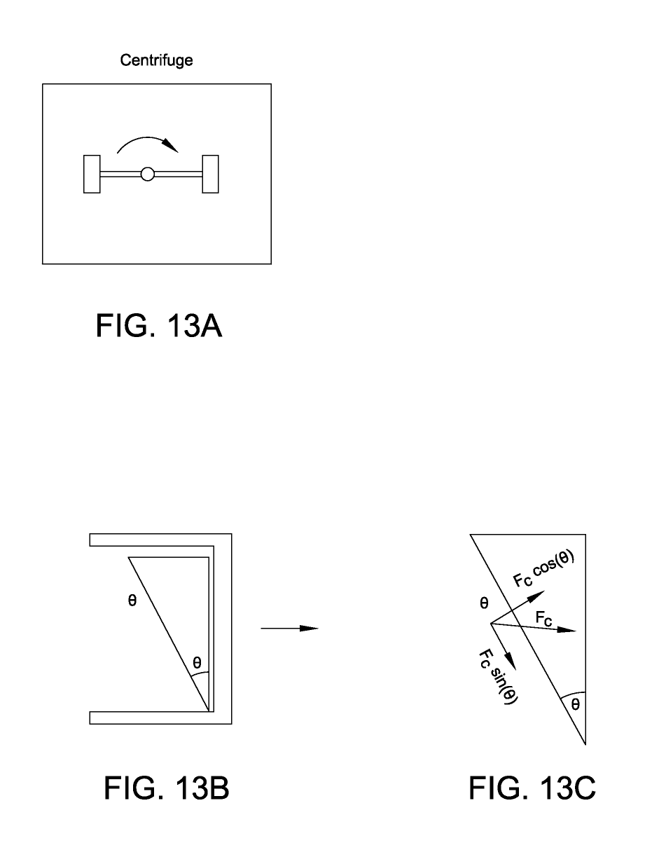

The substantially planar substrate can be disposed on a substrate which comprises a substantially planar region or substrate and a region which is not substantially planar substrate. A region which is not a substantially planar substrate could be: (a) a “capture feature” for capturing cells as they travel across the surface, which, in embodiments, optimizes the detection, e.g., optical detection, of unbound cells, (b) an “aggregate nucleation region” which, in embodiments is steeper than the substantially planar region and, relative to the direction traverse of cells across the substantially planar substrate, is upstream of it, which, in embodiments facilitates formation of aggregates, e.g., small aggregates, to more quickly clear off the substantially planar region for negative samples, and (c) a negative readout region which may or may not be part of the substantially planar region, and which, in embodiments, is where aggregates, e.g., large aggregates, to traverse to. FIGS. 13B-13C show exemplary substantially planar substrates.

The methods, devices and kits of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., SEQ ID NO: 1-SEQ ID NO: 37 are termed substantially identical.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid (SEQ ID NO:1) molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

### Forward Typing, Minor Antigen Typing and Extended Phenotyping

In one aspect, the invention provides methods, devices and kits for evaluating a sample for a red blood cell antigen, e.g., forward typing, minor antigen typing, or extended phenotyping. The method includes:

(a) contacting a red blood cell antigen binding agent, e.g., an anti-red blood cell antigen antibody, disposed on a surface (e.g., a functionalized surface as described herein) with the sample, e.g., a sample containing one or more red blood cells, under conditions sufficient for the formation of a complex between said red blood cell antigen binding agent, e.g., anti-red blood cell antigen antibody, and a red blood cell in said sample to occur, wherein said red blood cell comprises the red blood cell antigen (referred to herein as “complexed cells”);

(b) separating the complexed cells, e.g., by causing differential migration of red blood cells not complexed with said red blood cell antigen binding agent, e.g., anti-red blood cell antigen antibody (“uncomplexed cells”), relative to the complexed cells, across said substrate, wherein a change, e.g., an increase or decrease, in the amount of complexed and/or uncomplexed red blood cells, is correlated with the amount of said red blood cell antigen in said sample, thereby evaluating a sample for a red blood type antigen.

In an embodiment, the red blood cell antigen is a blood-type antigen, e.g., an A, B, AB or D antigen. In one embodiment, the method is a forward typing method, e.g., comprises the detection of a red blood cell antigen chosen from an A, B, or D antigen.

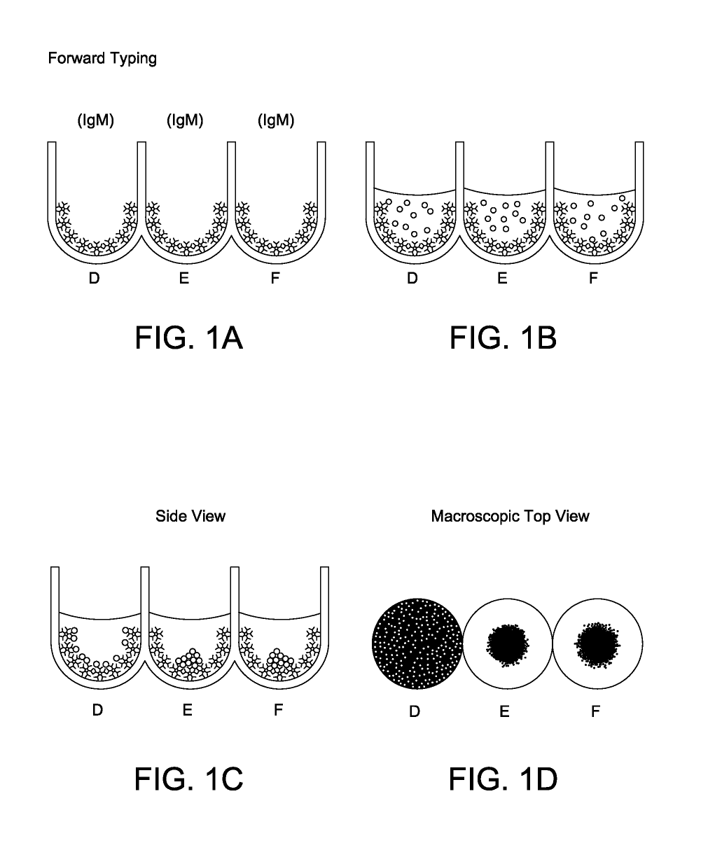

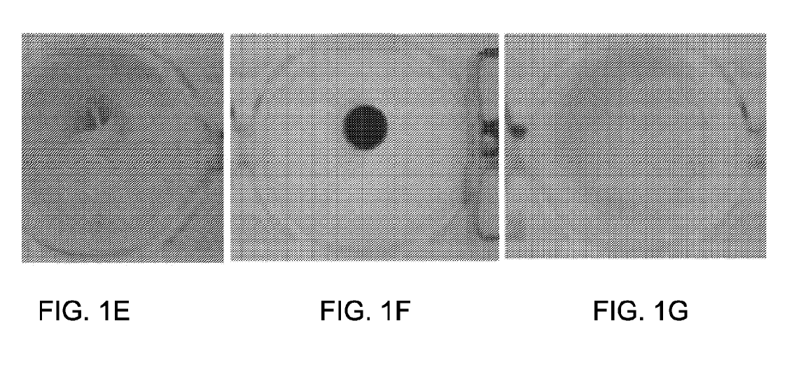

An embodiment of a forward typing assay is depicted in FIGS. 1A-1D.

Referring to FIG. 1A, a side view of three forward typing, U-shaped wells labeled D, E, and F is depicted. Each well is modified to contain a red blood cell binding agent disposed on (e.g., covalently or non-covalently bound to) its inner surface. In one embodiment, the red blood cell binding agent is an anti-red blood cell antigen antibody (e.g., an IgG or an IgM (as shown), or a combination thereof). In other embodiments, the red blood cell antigen binding agent can be a molecule that binds to a red blood cell antigen, e.g., a protein, a peptide or a carbohydrate. In other embodiments, the red blood cell antigen binding agent is a plant-derived binding agent, e.g., a lectin. In the embodiments shown in FIGS. 1A-1D, each well contains a different IgM antibody, e.g., an antibody against antigen D, E, and F disposed on the inner, lower portion of the well. A sample, e.g., plasma, serum or whole blood sample containing red blood cells (depicted as open circles in FIG. 1B), is added under conditions sufficient for the formation of a complex between said red blood cell antigen binding agent, e.g., anti-red blood cell antigen antibody, and a red blood cell in said sample to occur (referred to herein as “complexed cells”). In certain embodiments, the complexed cells are separated from the uncomplexed cells, e.g., by causing differential migration of red blood cells not complexed with said red blood cell antigen binding agent, e.g., anti-red blood cell antigen antibody (“uncomplexed cells”), relative to the complexed cells, across said substrate. The formation of complexed cells is represented well D in FIG. 1C. The positive readout is represented as a uniform distribution of the complexed cells across the inner surface of the well, represented in FIG. 1D as a homogeneous distribution across the entire top view of well D. Negative readouts are shown in a side view of wells E and F, depicted as an aggregate of uncomplexed cells. A top view of the negative readout is shown in schematic form in FIG. 1D, where the aggregated, uncomplexed cells are clustered in the center portion of the wells. FIGS. 1E and 1G show representative positive readouts, and FIG. 1F shows a representative negative readout for the forward typing assays described herein.

In other embodiments, the red blood cell antigen is chosen from at least 1, 2, 3, 4, 5, 6, 7, 8, 9, 10, 11, 12 or all of the RBC antigens provided in Table 1. In one embodiment, the red blood cell antigen is a minor antigen. In one embodiment, the red blood cell antigen is chosen from one, two, three, four, five, six, seven, eight, nine, ten, eleven, twelve, or more, or all of: a Rhesus antigen, e.g., one or more or all of D, C, c, E, or e; an MNS antigen, e.g., one or more or all of M, N, S, or s; a Kidd antigen, e.g., one or both of Jk^{a} or Jk^{b}; a Duffy antigen, e.g., one or both of Fy^{a} or Fy^{b}; a Kell antigen, e.g., one or both of K or k; a Lewis antigen, e.g., one or both of Le^{a} or Le^{b}; or aP antigen, e.g., P1. In certain embodiments, the red blood cell antigen analyzed includes at least the following RBC antigens: (1) D, C, E, e, c, and K; (2) D, C, E, e, c, K, Fy^{a} and Jk^{a}; or (3) D, C, E, e, c, K, Fy^{a}, Fy^{b}, Jk^{a}, Jk^{b}, S, and s.

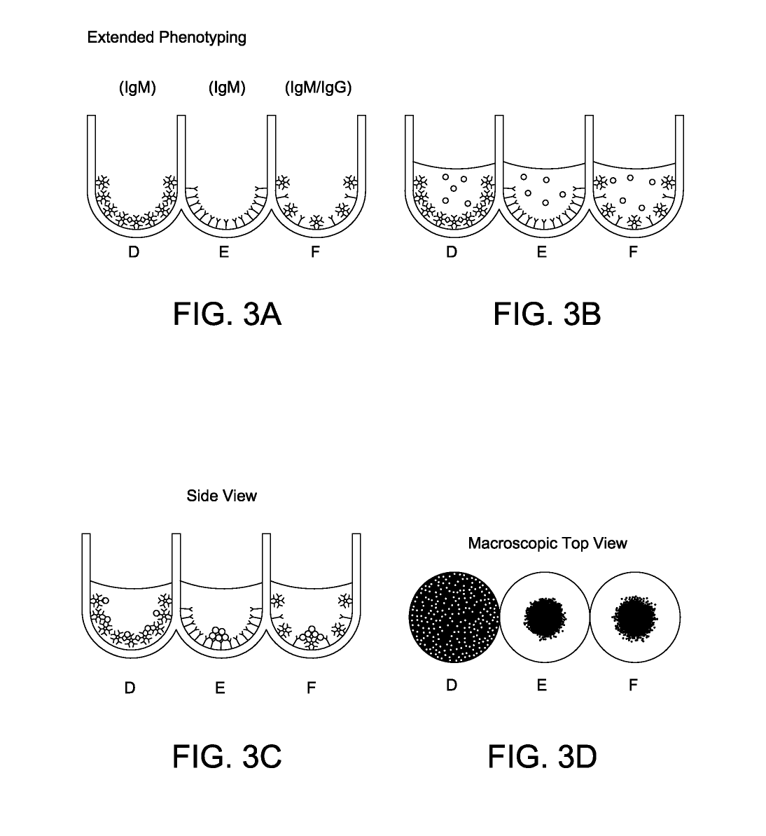

FIGS. 3A-3D provide a schematic of one embodiment of extended phenotyping assays. Similar to the forward typing assays, three wells, labeled D, E and F, are depicted, each one containing a different red blood cell antigen binding agent. The red blood cell antigen binding agent can be any molecule that binds to a red blood cell antigen, e.g., a protein, a peptide or a carbohydrate. In one embodiment, the red blood cell antigen binding agent is an anti-red blood cell antigen antibody (e.g., an IgG or an IgM, or a combination thereof). In other embodiments, the red blood cell antigen binding agent is a plant-derived binding agent. In the embodiments shown in FIGS. 3A-3D, each well contains a different IgM antibody (depicted as a pentameric structure in well D), IgG antibody (depicted as a “Y” in well E), or a combination of IgM and IgG antibodies (in well F), e.g., disposed on the inner, lower portion of the well. A sample, e.g., plasma, serum or whole blood sample containing red blood cells (depicted as open circles in FIG. 3B), is added under conditions sufficient for the formation of a complex between said red blood cell antigen binding agent, e.g., anti-red blood cell antigen antibody, and a red blood cell in said sample to occur (referred to herein as “complexed cells”). In certain embodiments, the complexed cells are separated from the uncomplexed cells, e.g., by causing differential migration of red blood cells not complexed with said red blood cell antigen binding agent, e.g., anti-red blood cell antigen antibody (“uncomplexed cells”), relative to the complexed cells, across said substrate. The formation of complexed cells is represented well D in FIG. 3C. The positive readout is represented as a uniform distribution of the complexed cells across the inner surface of the well, represented in FIG. 3D as a homogeneous distribution across the entire top view of well D. Negative readouts are shown in a side view of wells E and F, depicted as an aggregate of uncomplexed cells. A top view of the negative readout is shown in schematic form in FIG. 3D, where the aggregated, uncomplexed cells are clustered in the center portion of the wells

In an embodiment, the change, e.g., presence or absence, of detection uncomplexed cells is detected by in one or more of: a difference in the amount of the detection reagent (e.g., an increased or decreased presence of the detection reagent); a difference in the distribution of the detection reagent, e.g., on a surface; a difference in the amount of aggregation of the detection reagent; or a difference in the strength of adherence of the detection reagent to the rbcm preparation (e.g., as detected by optical trapping).

In one embodiment, the separation is effected by applying acceleration, e.g., centrifugal, fluid magnetic, electric or fluid, that causes migration of the complexed and uncomplexed cells.

In an embodiment, the surface is configured such that the applied acceleration results in migration of uncomplexed cells into an agglutination complex, e.g., at the bottom of a chamber (e.g., a well or a tube). In an embodiment, the detection of the presence of uncomplexed cells (e.g., a negative readout) is correlated with the absence of said anti-RBC antigen antibody and said sample. In certain embodiments, the negative readout is a button or a pellet. Exemplary schematics of negative readouts are shown in FIGS. 1D and 3D as samples E and F.

In one embodiment, the detection of the presence of complexed cells (e.g., a positive readout) is correlated with the presence of binding between said anti-RBC antigen antibody and said sample. In certain embodiments, the positive readout is detected as a haze. A schematic of the top views of the readout in chamber is depicted in FIGS. 1D and 3D, where a positive readout is detected as a haze in sample D of FIGS. 1D and 3D.

In an embodiment, the readout region is disposed in a chamber, e.g., a well or tube.

In an embodiment, the chamber is disposed on a carrier, e.g., a multi-chamber or multi-well plate, e.g., a 96 well plate.

In an embodiment, the angle between said carrier and the direction of force is non normal, e.g., between 25-5, 20-7.5, or 10 degrees.

### Reverse Grouping

In another aspect, the invention features a method of evaluating a sample for a red blood cell (RBC) antigen-specific antibody, e.g., reverse grouping or typing. The method comprises:

(a) contacting a rbcm preparation which comprises a red blood cell antigen, e.g., a blood group antigen, e.g., an A, B, or O antigen, disposed as a substrate of a surface, with sample, under conditions sufficient for the formation of a complex between said rbcm preparation and an anti-red blood cell antigen-specific antibody, e.g., anti-A, anti-B, or anti-C antibody, in said sample;

(b) providing indicator moieties, e.g., indicator cells (e.g., one or more red blood cells), positive for said red blood cell antigen, e.g., A+, B+, or O+ indicator cells, and an agent that can promote clumping between indicator moieties, e.g., indicator cells, under conditions sufficient for the formation of a complex, e.g., an immune complex, between said multi-valent binding agent and indicator moieties, e.g., indicator cells,

(c) applying acceleration, e.g., from centrifugal, gravitational, fluid magnetic, electric or fluid, force,

wherein said indicator moieties, e.g., indicator cells, e.g., by the distribution of indicator moieties, e.g., indicator cells, or by the strength of adhesion of unbound indicator moieties, e.g., indicator cells, to the substrate, indicate the presence or absence of said red blood cell antigen, thereby evaluating said sample.

In an embodiment, the multi-valent binding agent, e.g., an IgM antibody, binds a moiety that is present on said indicator cells but not present on said rbcm. In an example the moiety is a red blood cell antigen other than the red blood cell antigen being analysed. In an embodiment the moiety is other than a blood group antigen. In an embodiment the moiety is D antigen, and the multi-valent binding agent, e.g., an IgM antibody, is an anti-D antibody. In an embodiment the rbcm are negative for D antigen the indicator cells are positive for D antigen.

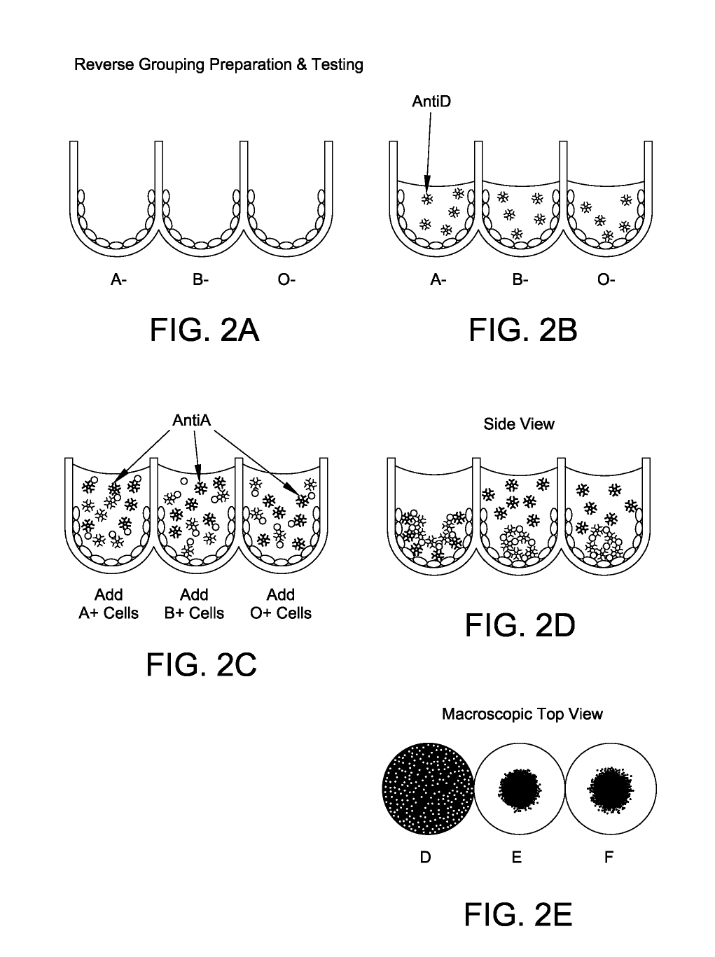

Referring to FIGS. 2A-2E, one embodiment of the reverse grouping preparation and testing is represented in schematic form. FIG. 2A provides three wells having different rbcm preparations disposed on the inner surface of the bottom of the wells to form a substrate. From left to right in FIG. 2A, each well is labeled A− (corresponding to the cells in the rbcm, e.g., A1 cells, RhD− cells); B− (corresponding to B cells, RhD− cells); and O− (corresponding to O cells, RhD− cells). A multi-valent binding agent, e.g., an IgM antibody, that binds a moiety that is present on said indicator cells, but not present on said rbcm is added in FIG. 2B, in this case, the multu-valent binging agent is an anti-D IgM antibody. In FIG. 2C, the sample is added and the corresponding indicator cell according to the rbcm preparation; e.g., from left to right, A+ cells, B+ cells, and O+ cells are added. The bound and unbound samples are separate, e.g., by centrifugation, and the results are depicted in FIGS. 2D and 2E (side and top views, respectively). The left hand well in FIGS. 2D and 2E depict a positive readout, showing a haze or uniform distribution of the indicator cells on the surface. The middle and right hand wells in FIGS. 2D and 2E show a representation of a negative readout, with a cluster of aggregated, non-bound antibody and cells at the bottom of the wells.

In certain embodiments, the rbcm preparation is disposed on a surface to form a substrate. In one embodiment, the rbcm preparation is bound (e.g., non-covalently or covalently) to a surface, e.g., a functionalized surface. For example, an rbcm preparation containing pre-selected red blood cells can be disposed (e.g., by centrifugation or gravitational settling) onto a surface capable of binding red blood cells. In embodiments, the rbcm preparation provides a substrate having a density of between 14000-24000, 24000-34000 and 34000-40000, cells/mm², e.g., 26,000 cells/mm² on the surface. Protocols and exemplary surfaces to be used in the methods are described herein.

In one embodiment, the indicator moieties, e.g., indicator cells, are present at a concentration that results in less than the entire substrate being covered with a monolayer of indicator moieties, e.g., indicator cells. E.g., the indicator moieties, e.g., indicator cells, are present in an amount that provides a sparse coating of the substrate. In embodiments, the indicator moieties, e.g., indicator cells, are present in an amount that results in coverage of less than or about 5%, 10%, 15%, 20%, 25% or 30% of the area of the substrate.

In other embodiments, the concentration of indicator moieties, e.g., indicator cells, is such that at least 30, 40, 50, 60, 70, 80, 90, or 100% of the substrate is covered with at least a monolayer of indicator moieties, e.g., indicator cells. In embodiments, the indicator moieties, e.g., indicator cells, are present at a concentration that results in the entire substrate being covered with at least a monolayer. In embodiments portions of the substrate are covered with more than one layer of indicator moieties, e.g., indicator cells, e.g., portions of the substrate are covered by a multilayer of indicator moieties, e.g., indicator cells. In embodiments, the detection indicator moieties, e.g., indicator cells, are present in an amount that is at least 10, 20, 30, 40, 50, 60, 70, 80, 90, or 100, and typically at least 50 times the amount that would give 20% coverage of the substrate with a monolayer.

In certain embodiments, the presence or absence of the anti-RBC antigen antibody in the sample is indicated by a parameter, e.g., a measurable parameter, related to the behavior of, or positional distribution of, indicator moieties, e.g., indicator cells. E.g., a preselected value for a parameter related to indicator moieties, e.g., indicator cells, is indicative of the presence or absence of the anti-RBC antigen antibody. The parameter can be, by way of example, the amount of the indicator moieties, e.g., indicator cells (e.g., an increased or decreased presence of the indicator moiety, e.g., indicator cell); the pattern of coverage of the substrate by the indicator moieties, e.g., indicator cells; the amount of coverage of the substrate by the indicator moieties, e.g., indicator cells; the distribution of the indicator moieties, e.g., indicator cells, e.g., on a substrate; the amount of aggregation of the indicator moieties, e.g., indicator cells; the strength of adherence of the indicator moieties, e.g., indicator cells, to the rbcm preparation (e.g., as detected by optical trapping).

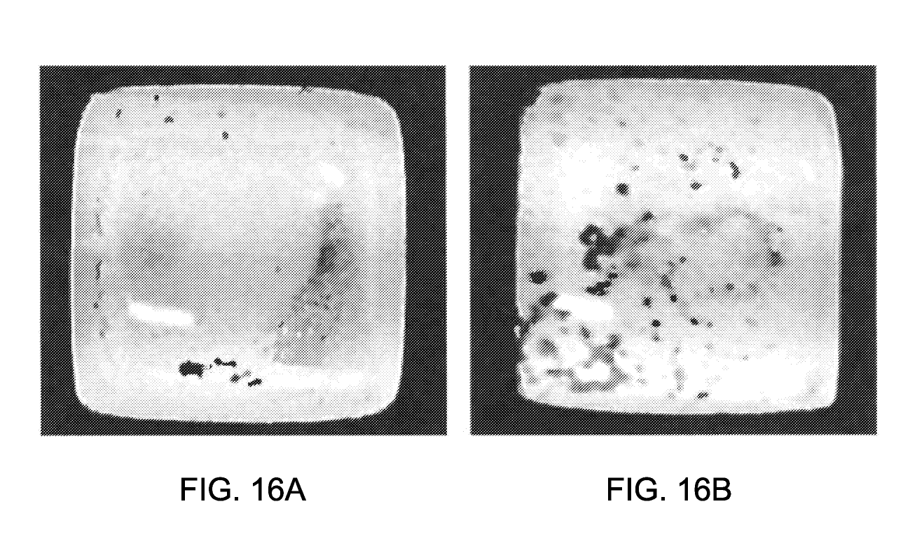

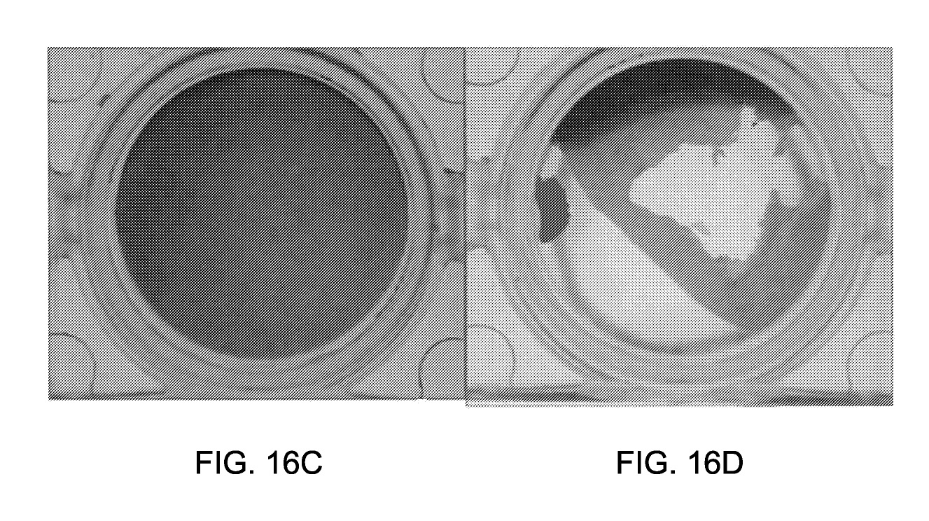

In one embodiment, the presence of the anti-RBC antigen antibody in the sample (or a positive readout) is detected by a uniform, homogenous distribution of the indicator moieties, e.g., indicator cells, on the substrate. In one embodiment, the positive readout is detected by having a coverage of the substrate by the indicator moieties, e.g., indicator cells, of at least 95%, 96%, 97%, 98%, 99% or 100% of the substrate area. An exemplary representation of a uniform distribution of the indicator moieties, e.g., indicator cells, is provided in FIG. 16C.

In another embodiment, the absence of the anti-RBC antigen antibody in the sample (or a negatve read out) is detected by a non-homogeneous distribution of the indicator moieties, e.g., indicator cells, on the substrate. In one embodiment, the negative readout is detected by having a coverage of the substrate by the indicator moieties, e.g., indicator cells, of less than 95%, 90%, 85%, 80%, 75%, 70%, 60%, 50%, 40% or 30% of the substrate area (e.g., relative to what would be covered in a positive sample). An exemplary representation of a non-homogeneous distribution of the indicator moieties, e.g., indicator cells, is provided in FIG. 16D. In one embodiment, the negative readout is detected as a localized concentration of indicator moieties, e.g., indicator cells—e.g., as a button or a pellet.

In certain embodiments, the difference in the indicator moieties, e.g., indicator cells, is detected by an increased or decreased formation of an aggregate.

In one embodiment, base units of non-bound indicator moieties, e.g., indicator cells, (indicator moieties, e.g., indicator cells, not bound to the rbcm preparation) form indicator moiety, e.g., indicator cell, complexes with one another, e.g., to form aggregates of non-bound indicator moieties, e.g., indicator cells. In embodiments, said aggregate comprises at least 2, 10, 20, 50, 100, 200, 1,000, 100,000, 1,000,000, 10,000,000 or 50,000,000 base units of indicator moieties, e.g., indicator cells. In one embodiment, the aggregate is of macroscopic dimension, e.g., an aggregate having an average dimension, e.g., at its largest point, of between 40-500 um, 75 um-1 mm, 100 um and 10 mm.

In an embodiment of the method, indicator moieties, e.g., indicator cells, traverse the substrate and collides with a second (or subsequent) indicator moieties, e.g., indicator cells—e.g., a indicator moieties, e.g., indicator cells, that traverses more slowly or is bound.

In one embodiment of the method, the indicator moieties, e.g., indicator cells, e.g., indicator moiety, e.g., indicator cell, complexes, e.g., an aggregate, that fails to bind to said first rbcm preparation, migrates across a substrate, e.g., into said first negative readout region of said carrier.

In other embodiments, the method further includes providing sufficient conditions, e.g., tangential velocity and sufficient time for indicator moieties, e.g., indicator cells, e.g., indicator moiety, e.g., indicator cell, complexes, e.g., an aggregate, that has not formed an immune complex to migrate across the substrate. In an embodiment, this results in uncovering substrate or reducing the amount of substrate covered by indicator moieties, e.g., indicator cells. In embodiments, the aggregate can migrate a first negative readout region.

In another embodiment, the difference in the indicator moieties, e.g., indicator cells, is detected by evaluating the strength of adherence of indicator moieties, e.g., indicator cells, to the rbcm preparation, e.g., to the substrate (e.g., as detected by optical trapping). In one embodiment, the displacement of non-bound indicator moieties, e.g., indicator cells, is evaluated by the optical trapping.

In one embodiment of the method, the presence or absence of indicator moiety, e.g., indicator cell, complexes, e.g., an aggregate, e.g., in a pre-selected location, is correlated with, respectively, the absence or presence, of said anti-RBC antigen antibody in said sample.

### Antibody Screening Methods and Devices

The present invention provides, at least in part, methods and devices for evaluating a sample, e.g., a plasma sample, from a subject, for detecting a target protein (e.g., an antibody (e.g., IgM-, IgG, IgE, an anti-red blood cell (RBC)-antigen antibody); an RBC antigen, e.g., an A, B, C, D, E, Rh, Kell antigen).

In one aspect, the invention features a method of evaluating a sample, e.g., a plasma sample, from a subject, for an anti-RBC-antigen antibody of G isotype. The method includes:

(a) contacting a first red blood cell membrane preparation (a rbcm preparation) comprising a first RBC antigen, e.g., a RBC antigen as described herein, with sample from said subject, under conditions sufficient for the formation of an immune complex between said first RBC antigen and anti-first-RBC-antigen antibody in said sample; and

(b) providing a detection reagent under conditions sufficient for the formation of a complex, e.g., an immune complex, between said detection reagent and an IgG antibody in said sample, said detection reagent comprising an IgG-specific binding moiety,

wherein the presence or absence of said detection reagent is correlated with the presence or absence of said anti-RBC antigen antibody in said sample, thereby evaluating a sample for an anti-RBC antigen antibody of G isotype.

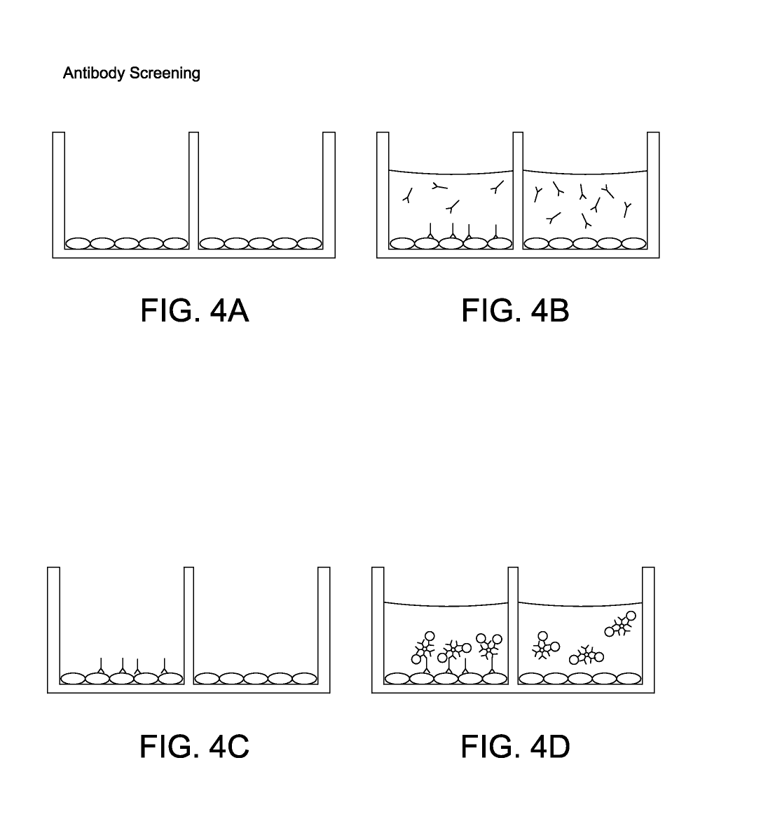

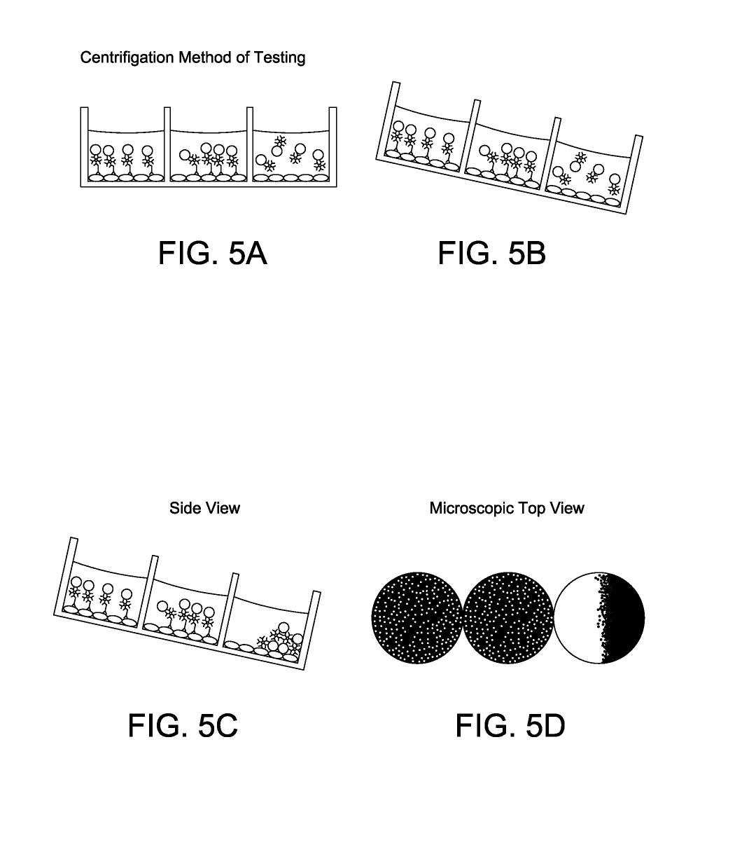

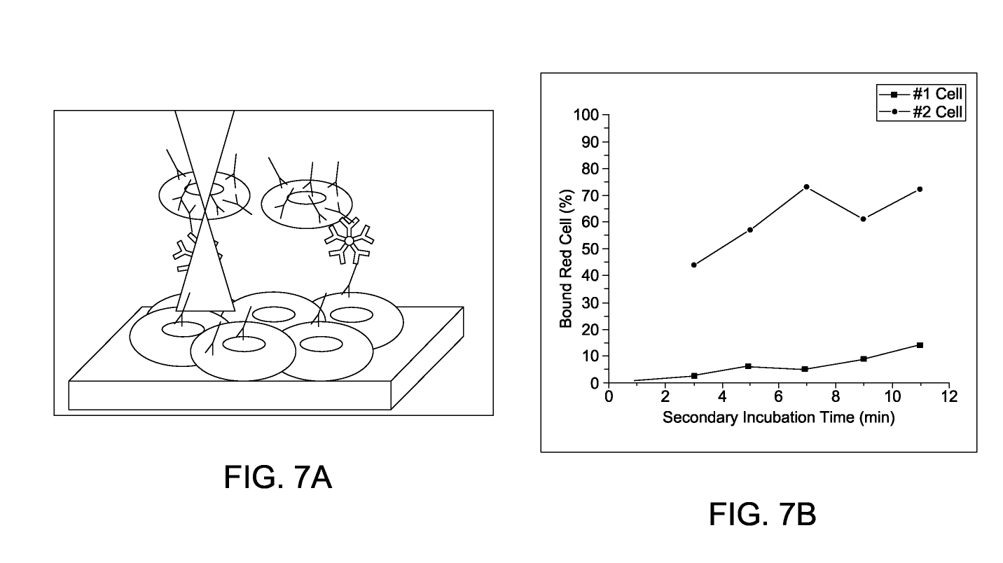

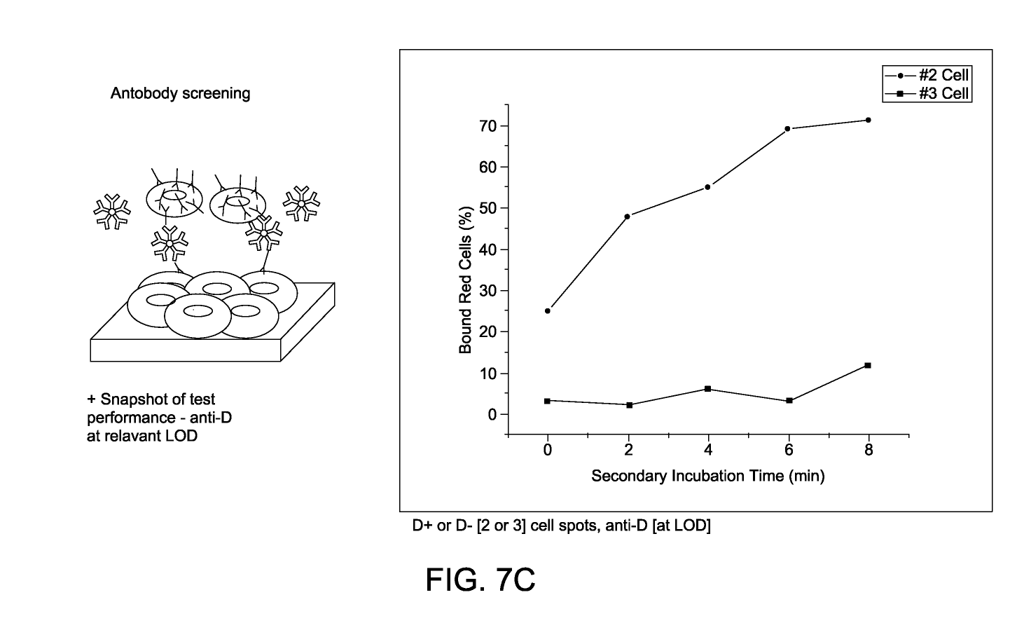

A schematic of an exemplary assay format is shown in FIGS. 4A-4D and FIG. 7A. In these embodiments, a red blood cell membrane preparation is bound to a surface and, optionally, lysed, thereby forming a substrate, depicted in FIG. 4A. A sample, e.g., a serum, plasma or whole blood sample, containing an anti-RBC antigen antibody of a G isotype (e.g., one or more anti-RBC antigen antibodies of a G isotype) is incubated with the red blood cell membrane preparation under conditions that allow for a formation of an immune complex between the RBC antigen and the anti-RBC antigen antibody of a G isotype (FIG. 4B). Unbound IgG can be reduced by one or more washing steps, depicted in FIG. 4C. A detection reagent that includes an IgG-specific binding moiety is added to the incubated sample, thereby allowing detection of the immune complex. In one embodiment, the detection reagent includes an IgG binding reagent (e.g., a monoclonal IgM class anti-human IgG from clone MS-278). The detection reagent can further include an indicator cell, optionally, having one or more IgG-specific binding agents depicted in FIG. 4D, e.g., an indicator Alba Bioscience IgG sensitized cells, thereby allowing measurement of the presence of IgG class antibodies which are specific to rbcm antigens from the plasma by way of detection of bound red blood cells, e.g., by detecting binding of the indicator cells to the test surface.

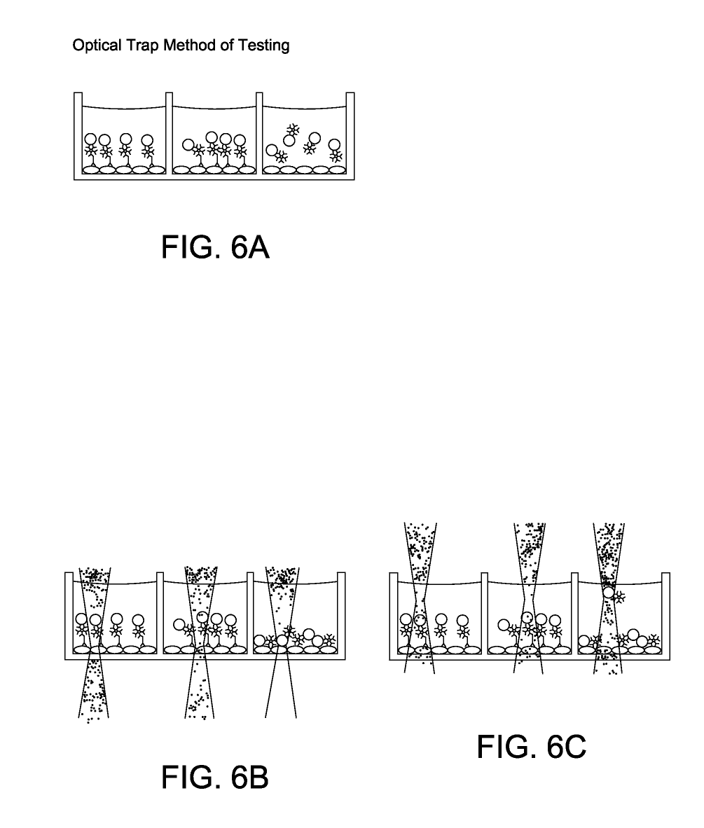

In another embodiment, detection of bound red blood cell can be effected by optical trapping, depicted in schematic form in FIGS. 6A-6C. Referring to FIG. 6A, three side views of wells containing a sample anti-RBC antigen antibody of a G isotype bound to the rbcm preparation are shown in the first and second wells (from left to right views of FIG. 6A). An unbound detection reagent is shown on the right-side well in FIG. 6A. FIGS. 6B-6C show the effect of optical trapping in displacing the unbound detection reagent (see right-hand panel in FIG. 6C), compared to the undisplaced, bound detection reagent at the bottom of the wells in the left-most and middle panels of FIG. 6C.

### IgG Binding Moieties

In one embodiment, IgG binding moieties that bind selectively and specifically to plasma IgG relative to the binding to the rbcm preparation are disclosed (see FIGS. 7B, 7C, 9, and 10, described in the Examples).

In certain embodiments, the IgG-specific binding moiety includes an antibody molecule that binds to an IgG, e.g., an antibody molecule that binds to a constant region (e.g., a heavy chain Fc region or a light chain constant region) of the IgG, or a heavy or light chain variable domain of the IgG. In one embodiment, the antibody molecule binds to an IgG constant region chosen from one, two, three or all four of IgG1, IgG2, IgG3, or IgG4. In another embodiment, the antibody molecule binds to a light chain constant region of an IgG chosen from, e.g., the (e.g., human) light chain constant regions of kappa or lambda.

As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example). In yet other embodiments, the antibody molecule has a heavy chain constant region chosen from, e.g., the heavy chain constant regions of IgM, IgG1, IgG2, IgG3, IgG4, IgA1, IgA2, IgD, and IgE. In another embodiment, the antibody molecule has a light chain constant region chosen from, e.g., the (e.g., human) light chain constant regions of kappa or lambda.

In one embodiment, the IgG-specific binding moiety includes an antibody molecule that binds to an IgG-common site in the constant region. In other embodiments, the IgG-specific binding moiety includes antibody molecule that binds to an IgG-common site in the light chain variable region. In other embodiments, the IgG-specific binding moiety includes an antibody molecule that binds to a light chain constant region.

In one embodiment, the IgG-specific binding moiety includes an antibody molecule that has one or more of the properties (e.g., binding properties) of monoclonal antibody MS-278 (e.g., the IgG-specific binding moiety comprises a monoclonal antibody MS-278, or an antigen binding fragment thereof). Monoclonal antibody MS-278 is a murine IgM from clone MS-278. It reacts with all four subtypes of human IgG. Monoclonal antibody (mAb) MS-278 can be obtained from Millipore Corporation.

In certain embodiments, the IgG-specific binding moiety, e.g., an antibody, e.g., a mAb, or an antigen binding fragment thereof, has one or more of the following properties: (i) it comprises mAb MS-278, or an antigen binding fragment thereof; (ii) it competes with mAb MS-278 for binding to IgG; (iii) it binds to an epitope bound by mAb MS-278 (e.g., the same or an overlapping epitope); (iv) it binds to rbcm preparations at a level which is no more than 1.2, 1.5, 1.75, 2, 3, 4 or 5 times that of mAb MS-278, e. g., as determined by an assay described herein; (v) it binds to IgG at a level which is at least 20, 30, 40, 50, 60, 70, 80, 90, or 100% of mAb MS-278, e.g. as determined by an assay described herein.

In one embodiment, the IgG-specific binding moiety has a specificity for plasma IgG relative to an rbcm preparation as shown for mAb MS-278 in FIG. 1B. In embodiments, the IgG-specific binding moiety shows a reduction in non-specific binding to the rbcm preparation of at least 10%, 20%, 30% or more compared to an IgG-specific antibody chosen from 16H8 [Immucor], rabbit polyclonal [Alba #Z356], rabbit polyclonal [Biotest #804501], material from cell line CG-7 [Sigma-Aldrich I6260], or goat polyclonal [Sigma-Aldrich #I2136].

In other embodiments, the IgG-specific binding moiety includes an anti-IgG light chain antibody molecule. In one embodiment, the anti-IgG light chain antibody molecule has one or more of the properties (e.g., binding properties) of an anti-light chain antibody chosen from Sigma-Aldrich #K4377 Cell Line KP-53, Sigma-Aldrich #L6522 cell line HP-6054, Sigma-Aldrich #K3502—polyclonal, or Sigma-Aldrich #L7646—polyclonal). In certain embodiments, the anti-IgG light chain antibody molecule has one or more of the following properties: (i) competes with the anti-IgG light chain antibody molecule for binding to IgG; (ii) binds to an epitope bound by the anti-IgG light chain antibody molecule (e.g., the same or an overlapping epitope); (iii) its level of binding to a rbcm preparation is less than 1, 2, 5, 10, 25, or 50% of the binding of the anti-IgG light chain antibody molecule to rbcm preparation; (iv) it binds to IgG with an affinity that is at least 20, 30, 40, 50, 60, 70, 80, 90, or 100% of the affinity with which the anti-IgG light chain antibody molecule binds IgG; or (v) displays specific binding to and IgG of at least 10, 20, or 30% and non specific binding of less than 2 or 5%, e.g., as determined by a method described herein.

The binding properties of an IgG-specific binding moiety can be measured by methods know in the art, e.g., one of the following methods: BIACORE. analysis, Enzyme Linked Immunosorbent Assay (ELISA), x-ray crystallography, sequence analysis and scanning mutagenesis. The ability of an IgG-specific binding moiety to selectively bind to plasma IgG relative to an rbcm preparation can be tested by the assays described herein (e.g., Example 1). The binding interaction of IgG-specific binding moiety and a target (e.g., IgG or an IgG mimic) can be analyzed using surface plasmon resonance (SPR). For example, SPR can be used to identify the binding epitope of the IgG-specific binding moiety. SPR or Biomolecular Interaction Analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (Kd), and kinetic parameters, including Kon and Koff, for the binding of a molecule to a target. Such data can be used to compare different molecules. Information from SPR can also be used to develop structure-activity relationships (SAR). For example, the kinetic and equilibrium binding parameters of different antibody molecule can be evaluated. Variant amino acids at given positions can be identified that correlate with particular binding parameters, e.g., high affinity and slow Koff. This information can be combined with structural modeling (e.g., using homology modeling, energy minimization, or structure determination by x-ray crystallography or NMR). As a result, an understanding of the physical interaction between the protein and its target can be formulated and used to guide other design processes.

The binding properties, e.g., specificity, of the IgG-specific binding moiety for plasma IgG relative to the rbcm preparation can be evaluated as follows. Candidate anti-IgG (such as material from cell line MS-278, 16H8 [Immucor], rabbit polyclonal [Alba #Z356], rabbit polyclonal [Biotest #804501], material from cell line CG-7 [Sigma-Aldrich I6260], goat polyclonal [Sigma-Aldrich #I2136]) can be first tested for its ability to agglutinate human red blood cells coated with human or humanized IgG (such as Alba #Z441). Alternatively, if such red blood cells are not commercially available, human plasma units that contain abnormal antibodies specific to human blood groups can be acquired from local and regional blood centers. For instance, a plasma unit that contains anti-D may be used to functionalized D+ red blood cells with IgG. The cell should be washed using processes known to those skilled in the art to remove unbound IgG.

Next, the functionalized red cells are dispersed with varying concentration of candidate anti-IgG and graded for hemagglutination by eye. The titration produces a bell curve and the peak of this curve represents the optimal concentration of the anti-IgG. Control experiments with non-functionalized red cells can be conducted simultaneously to ensure reaction specificity. Once the reaction specificity and optimal concentration have been determined, optical trapping based experiments can be used to evaluate binding properties.

In one embodiment, an optical trap can be used to probe the binding of indicator cells to the prepared surface. Briefly, a collimated 0.5 W 1064 nm continuous laser beam (via a laser such as IPG # YLR-25V-SM-NC) with a diameter of 7-12 mm (measured at the back aperture of the objective) is shone through a Nikon Plan APO 40 X (NA 0.95) objective mounted in a research grade inverted microscope (Nikon TE-200 or Olympus IX2 series). The beam diameter can be adjusted via two lenses [Thorlabs LB1309 and LB 1630]. Those skilled in the art should be familiar with the importance of optical alignment and such. The sample should be maneuvered via a precision stage. Optical trapping techniques, including instrument design considerations, position detection schemes and calibration techniques are reviewed in Neuman, K. C. and Block, S, (2004) Rev. Sci. Instrum. 75(9):2787-2809, the contents of which are hereby incorporated by reference in their entirety. Further experimental conditions for testing the candidate anti-IgG antibodies are described in detail in Example 1 and summarized briefly herein. The candidate anti-IgG antibody (at its optimal concentration) is incubated over a red blood cell coated-surface at various temperatures and times, for example, 20° C. for 10 minutes. The red cell surface can be washed with normal saline to remove unbound anti-IgG. Next, IgG-sensitized red cells can be added to the test well and allowed to sediment to the test surface. Binding can then be probed with optical trapping. In one embodiment, the anti-IgG antibody yields fewer than 15% bound cells when probed with an optical trap.

Protocols for preparing suitable surfaces coated with red blood cell membrane preparations are described in Example 1. Briefly, a suitable surface should be positively charged at neutral pH and substantially free of surface contamination. Any number of surface treatments can be used. For example, a native polystyrene surface can be rendered positively charged via a molecule with a hydrophobic character and an appropriate electrostatic character (for instance, poly-L-lysine). Silica can be rendered positively charged via an amine terminated silane (such as aminopropyltriethoxysilane—APTES) or similar agents. The uniformity of the film can be probed by exposing the surface to amine reactive fluorescent tags such as fluorescein isothiocyanate and examining with fluorescence microscopy. Red blood cells can be deposited and lysed as described in Example 1.

In certain embodiments, the candidate anti-IgG antibody must also bind appropriately to true IgG at low concentrations (i.e., enable an assay with a relevant limit of detection). In order to determine the limit of detection, proficiency standards that represent a minimum level of performance (as defined by existing commercially available tests) can be obtained. For instance, an anti-D proficiency kit can be obtained from Alba Bioscience (#Z261). If such standards are unavailable, plasma units containing abnormal antibodies can be obtained from local and regional blood centers. These plasma units can be titrated (i.e., diluted with normal human plasma) to various levels and tested on relevant commercial platforms until a defined threshold for detection is obtained.

Once these benchmarks are established, the titrated plasma sample can be incubated over the red blood cell coated surface in conditions known to those skilled in the art. An exemplary incubation condition is 37° C., 15 minutes, 1:1 ratio 0.025 M NaCl (Low Ionic Strength Saline—LISS). The test can be run in parallel such that red blood cell surfaces expressing and not expressing the antigen corresponding to the antibody specificity are examined. The test surfaces can be washed with normal saline until sufficiently free of unbound IgG. Candidate anti-IgG can be blended with the IgG-coated red cells and then dispersed over the test surfaces. Three minutes are allotted for sedimentation of the cells to the test surfaces.

After the three minute sedimentation time, an appropriate candidate anti-IgG antibody typically yields specific binding greater than 20% and nonspecific binding under 10%. The specific binding signal typically continues to increase to a level of 40% or more bound red cells after six minutes. The nonspecific signal should not appreciably (i.e., surpass 20%) increase.

Similar protocols can be used to evaluate candidate light-chain specific anti-IgG antibodies. Candidate light-chain specific anti-IgG antibodies (such as Sigma-Aldrich #K4377 Cell Line KP-53, Sigma-Aldrich #L6522 cell line HP-6054, Sigma-Aldrich #K3502—polyclonal, Sigma-Aldrich #L7646—polyclonal) are titrated to an appropriate level. A titration can be conducted by first sensitizing human red blood cells with a fully human IgG. Such samples can be obtained from local and regional blood centers. After the cells are sensitized, they are washed to remove unbound IgG. Next, the cells are incubated with the light chain specific anti-IgG (at some concentration) and then washed to remove unbound anti-IgG. As a light chain specific anti-IgG is typically unable to agglutinate native red blood cells, a third antibody is needed to bind to the heavy chain of the light chain specific anti-IgG. If the light chain specific anti-IgG is murine, a murine specific anti-IgG (such as Sigma #M1397) can be used. Control experiments to evaluate trivial cross-species reactivity can also be conducted. The third antibody is added to the sensitized red blood cells at various concentrations (i.e., titrate) and agglutination graded. Such experiments will yield a bell curve for each concentration of light chain specific anti-IgG. These curves can be condensed to a master curve and the peak amplitude represents the ideal concentration of light chain specific anti-IgG.

Once the optimal concentration of light chain specific anti-IgG is identified, the indicator red blood cell can be enzymatically treated to reduce the negative charge to enable binding. Such cells may be obtained from commercial sources (Alba #Z452) or prepared via those skilled in the arts (numerous procedures reside within the public domain—for instance, the AABB Technical Manual). The cells can be functionalized with IgG via a fully human antibody. Such samples can be obtained from local or regional blood centers. As the enzyme treatment often allows IgG to agglutinate red blood cells, high titer antibody units (i.e., units that display high agglutinating power at strong dilutions) can be used in an attempt to saturate all antigen sites on the red blood cell surfaces. If agglutins form during the sensitization of the red blood cells, they can be disrupted via vigorous pipetting. After the cells are sensitized, they should be washed via saline to remove unbound IgG.

The candidate light chain specific anti-IgG antibody can then be incubated over the test surface at 20° C. for 10 minutes. The test surface should not possess the antigen system used to label the indicator red cells (i.e., if anti-D is used to sensitize the indicator red blood cells, D− cells should be used on the test surface). The test surface can be washed with normal saline. Next, IgG sensitized enzymatically treated red cells are dispersed over the surface and allowed to sediment. Binding should be probed with optical trapping. In one embodiment, the anti-light chain specific anti-IgG antibody yields binding less than 3%.

If such a candidate light chain specific antibody is encountered, its specific binding activity can also be demonstrated. This can be accomplished by incubating an appropriate reference sample (see discussion above on relevant limits of detection) over the test surface and then washing the surface to remove unbound IgG. The antibody system of interest can be chosen such that: 1) The substrate does not possess the antigen used to label the indicator cell (i.e., if anti-D is used to label the indicator, D− cell should be used on the substrate; and/or 2) The indicator does not possess the antigen used to probe specific binding (i.e., if the plasma unit contains anti-c, the indicator should be c−). These precautions ensure that specific binding can only occur through the anti-light chain antibody.

The anti-light chain antibody is then dispersed with the prepared indicator cells and then added to the test surface and three minutes allotted for sedimentation. The system is then probed with optical trapping. In one embodiment, the anti-light chain antibody displays specific binding exceeding 10% and non-specific binding less than 2%.

The IgG-specific binding moiety can be modified to be coupled (e.g., covalently or non-covalently) to a detection reagent, e.g., an indicator moiety such as a fluorescent, cellular, or colloidal indicator. In one embodiment, the detection reagent includes a red blood cell, and optionally, a second antibody molecule that binds to the IgG-specific binding moiety (e.g., an IgG sensitized red blood cell obtained from Alba Bioscience). In such embodiments, a positive readout is the formation of red blood cell aggregates. In other embodiments, the detection reagent (e.g., a second antibody molecule) is directly or indirectly labeled with a detectable substance to facilitate detection of the bound or unbound antibody. Suitable detectable substances include various enzymes, prosthetic groups, fluorescent materials, luminescent materials and radioactive materials. A variety of suitable fluorescers and chromophores are described by Stryer (1968) Science, 162:526 and Brand, L. et al. (1972) Annual Review of Biochemistry, 41:843-868. The binding agents can be labeled with fluorescent chromophore groups by conventional procedures such as those disclosed in U.S. Pat. Nos. 3,940,475, 4,289,747, and 4,376,110. Other examples of fluorescers include fluoresceins, rhodamines, and naphthylamines. Procedures for labeling polypeptides with the radioactive isotopes (such as 14C, 3H, 35S, 125I, 99mTc, 32P, 33P, and 131I) are generally known. See, e.g., U.S. Pat. No. 4,302,438; Goding, J. W. (Monoclonal antibodies: principles and practice: production and application of monoclonal antibodies in cell biology, biochemistry, and immunology 2nd ed. London; Orlando: Academic Press, 1986. pp 124-126).

Solid substrates for antibody screening are known in the art. For example, solid phase blood typing using red cell membrane preparations are described in U.S. Pat. No. 5,030,560, incorporated herein by reference. Other solid support substrates include array systems, e.g., microarrays, as described in WO 2008/122793, also incorporated by reference. In embodiments where screening for blood antibodies is desired, a plurality of blood group antigens (e.g., rbcm preparations) which are capable of binding specifically to an anti-RBC antigen antibody are immobilized on a substrate, e.g., a microarray substrate, at pre-defined positions. The sample, e.g., plasma sample, is added under conditions suitable for specific binding of the sample antibodies to the blood group antigens. The presence of the bound antibodies is detected in the microarray.

### Mimic-Optimized Rbcm Preparations

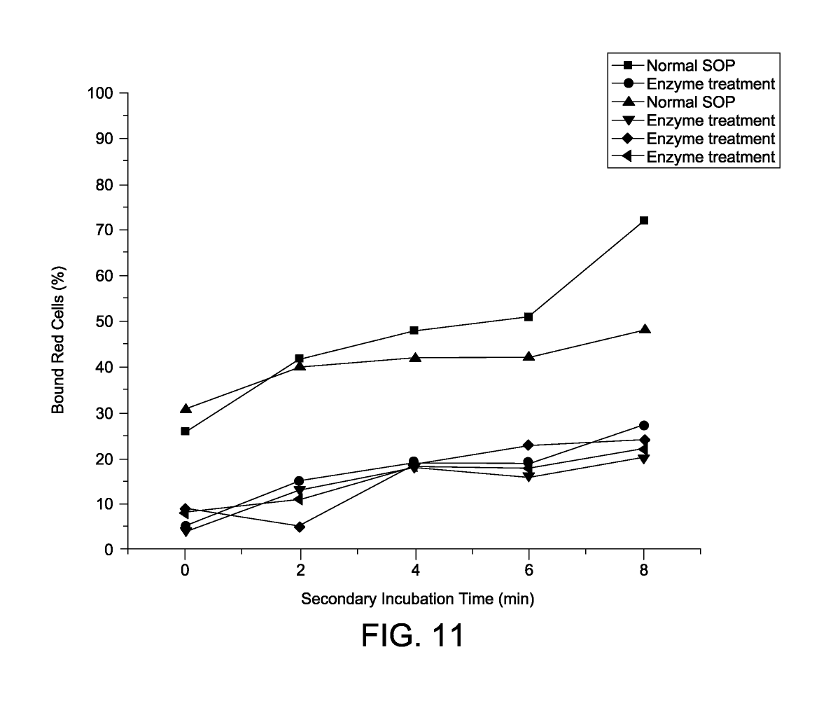

Alternatively or in combination with the methods described herein, non-specific binding caused by the lysed red blood cell membrane preparation can be reduced by an agent that disrupts the IgG mimic (e.g., an enzyme that cleaves IgG) present on the rbcm preparations, thereby producing a mimic-optimized rbcm preparation. In one embodiment, the agent is an enzyme, e.g., a cysteine proteinase, with specificity for immunoglobulin G. In one embodiment, the enzyme preferentially cleaves human IgG in the hinge region with a high degree of specificity. In one embodiment, the enzyme is an immunoglobulin-degrading enzyme of S. pyogenes (e.g., IdeS). The IdeS enzyme is described in von Pawel-Rammingen, et al. (2002) EMBO Journal 21 (7): 1607-1615 and WO 03/051914, the contents of which are incorporated by reference. FabRICATOR® (Genovis #A0-FR1-020) is commercially available. Red blood cell membrane preparations pre-treated with the immunoglobulin-degrading enzyme (e.g., IdeS) show a significant decrease in non-specific binding to the red cell membrane preparation, compared to non-treated red cell membrane preparations (FIG. 11). Experimental conditions for pre-treatment of the rbcm preparation with the immunoglobulin-degrading enzyme are provided in Example 1. Briefly, a rbcm preparation (e.g., lysed red cells deposited onto a test surface) was incubated in the presence of FabRICATOR® under conditions suitable to effect FabRICATOR®'s lysing activity. Control rbcm preparations were lysed deposited cells that not exposed to FabRICATOR®. Non-specific binding of an anti-IgG antibody (e.g., Alba #Z356) was detected. The results are shown in FIG. 11.

### Methods and Devices for Improving the Performance of Solid-Phase Capturing Methods

In another aspect, the invention provides a method of, or device for, evaluating a sample for an analyte. The method includes:

(a) contacting a capture agent (e.g., an antibody, an antigen (e.g., an RBC antigen, an rbcm preparation, an optimized rbcm preparation) with sample, under conditions sufficient for the formation of a complex between a capture agent, and said analyte (e.g., an antigen, an antibody or other protein having specific binding for said capture agent, e.g., in an anti-red blood cell antibody and a rbcm preparation) in said sample,

wherein, said capture agent is disposed on a substantially planar substrate, and the angle between said substantially planar substrate and the direction of applied force, e.g., centrifugal, gravitational, fluid magnetic, electric or fluid, force, that causes migration of detection reagent, is non-orthogonal or other than 90 degrees (theta, the angle formed by the substantially planar substrate and a line perpendicular to the direction of centrifugal force, is nonzero);

(b) providing a detection reagent (wherein said detection reagent can comprise a cell, e.g., a red blood cell, e.g., as an indicator moiety) under conditions sufficient for the formation of a complex, e.g., an immune complex, between said detection reagent and analyte, e.g., anti-capture agent antibody in said sample,

(c) applying acceleration, centrifugal acceleration, at said angle such that detection reagent that does not bind to said capture agent migrates across said substrate or surface, e.g., substantially planar substrate oe surface,

wherein, the presence or absence of detection reagent, e.g., in a preselected location, is correlated with the presence or absence of said analyte, e.g., anti-capture agent antibody, in said sample, thereby evaluating a sample for an analyte.

In one embodiment, the capture agent is a RBC antigen, e.g., at least 1, 2, 3, 4, or all of the RBC antigens provided in Table 1. Exemplary RBC antigens include at least 1, 2, 3, 4, or all of the following RBC antigens: a Rhesus antigen, e.g., one or more or all of D, C, c, E, or e; a MNS antigen, e.g., one or more or all of M, N, S, or s; a Kidd antigen, e.g., one or both of Jka or Jkb; a Duffy antigen, e.g., one or both of Fya or Fyb; a Kell antigen, e.g., one or both of K or k; a Lewis antigen, e.g., one or both of Lea or Leb; or P antigen. In another embodiment, the capture agent is a pathogen antigen, e.g. a viral antigen, e.g., a viral antigen chosen from one or more of human immunodeficiency (HIV) virus, hepatitis B virus (HBV), syphilis, human T-lymphotropic virus (HTLV), hepatitis C virus (HCV), or syphilis. Exemplary pathogen antigens include an HIV 1/2 antigen, e.g., p24, p15, p17, gp36, or gp 41; a Hep B antigen, e.g., HepBsAg, or HepBcAg; or a Syphilis antigen, e.g., TmpA, p15, 17, or 47.

In other embodiments, the capture agent is an anti-RBC antigen antibody, e.g., an antibody against at least 1, 2, 3, 4, or all of the RBC antigens provided in Table 1, e.g., an RBC antigen described herein. In yet other embodiments, the capture agent is an anti-pathogen antigen antibody, e.g., an antibody against a viral antigen, e.g., a viral antigen chosen from one or more of human immunodeficiency (HIV) virus, hepatitis B virus (HBV), syphilis, human T-lymphotropic virus (HTLV), hepatitis C virus (HCV), or syphilis, e.g., a viral antigen as described herein.

In yet other embodiments, the capture agent is a CMV, WNV, HTLV-1 and 2, or platelet antigen, or an antibody against same.

In certain embodiment, target antibodies are obtained from a blood sample, and testing is carried out against an array of uniquely treated surfaces to determine an antibody profile. In one embodiment, the target antibodies are obtained from a blood sample for the purposes of detecting viral infection. Antigens that occur on the surface of a given virus can be immobilized on the surface (i.e., solid phase) thereby being able to capture the specific antibody to that virus. In addition, particles coated with antibodies complementary to another region of the virus antibody can be present in the test, such that in the presence of the target virus antibody, immobilization of particles may occur, signaling the presence of the antibody in the blood sample. Such measurements are performed in order to diagnose infection, or quantify target antibody concentration, with suitable controls.

In certain embodiments, two different forces are applied, a first force to provide a force substantially normal to the substrate or surface, e.g., substantially planar substrate and a second force to provide an additional force tangential to said substrate or surface, e.g., substantially planar substrate. In one embodiment, the first force, e.g., a magnetic force, is applied to produce force normal to said substantially planar substrate on a detection reagent complex or aggregate, and a second force, e.g., fluid force, is applied to produce force tangential to said substantially planar substrate on a detection reagent complex or aggregate.

In another aspect, the invention features a method of, or device for, evaluating a sample, e.g., a plasma sample, from a subject, for an anti-RBC antigen antibody. The method includes:

(a) contacting a first red blood cell membrane (rbcm) preparation with sample from said subject, under conditions sufficient for the formation of an immune complex between a first RBC antigen and anti-first RBC antigen antibody in said sample,

wherein, said first rbcm preparation is disposed on a substrate, e.g., a substantially planar substrate, and the angle between said substrate and the direction of applied force, e.g., centrifugal or, gravitational, fluid magnetic, electric or fluid, force, that causes migration of detection reagent, is non-orthogonal or other than 90 degrees (in other words, theta, the angle formed by the substantially planar substrate and a line perpendicular to the direction of centrifugal force, is nonzero);

(b) providing a detection reagent under conditions sufficient for the formation of a complex, e.g., an immune complex, between said detection reagent and an anti-RBC antigen antibody in said sample,

(c) applying force, e.g., centrifugal force, at said angle such that detection reagent that does not bind to said first rbcm preparation migrates across said substrate,

wherein the presence or absence of detection reagent, e.g., in a preselected location, is correlated with the presence or absence of said anti-first RBC antigen antibody in said sample, thereby evaluating a sample for an anti-RBC antigen antibody.

In other embodiments, the method or the device further includes:

(d) contacting a second rbcm preparation with sample from said subject, under conditions sufficient for the formation of an immune complex between a second RBC antigen and anti-second RBC antigen antibody in said sample,
wherein, said second rbcm preparation is disposed on a substrate, e.g., a substantially planar substrate, and the angle between said substrate and the direction of applied force, e.g., centrifugal, gravitational, fluid magnetic, electric or fluid, force, that causes migration of detection reagent, is non-orthogonal or other than 90 degrees;

(e) providing a detection reagent under conditions sufficient for the formation of a complex, e.g., an immune complex, between said detection reagent and an anti-RBC antigen antibody in said sample,

(f) applying centrifugal force at said angle such that detection reagent that does not bind to said second rbcm preparation migrates across said substrate,

wherein the presence or absence of detection reagent, e.g., in a preselected location, is correlated with the presence or absence of said anti-second RBC antigen antibody in said sample,

thereby evaluating a sample for an anti-second blood-type-antigen antibody of pre-selected isotype.

In certain embodiments, steps (a) and (d) are performed at least partially simultaneously. In other embodiments, steps (b) and (e) are performed at least partially simultaneously. In yet other embodiments, steps (c) and (f) are performed at least partially simultaneously.

In other embodiments, the method or the device further includes evaluating said sample for an N^{th}, e.g., third, anti-RBC antigen by:

(g) contacting an N^{th}, e.g., third, rbcm preparation with sample from said subject, under conditions sufficient for the formation of an immune complex between an N^{th}, e.g., third, RBC antigen and anti- an N^{th}, e.g., third, RBC antigen antibody in said sample,
wherein, said N^{th} rbcm preparation is disposed on a substrate, e.g., a substantially planar substrate, and the angle between said substantially planar substrate and the direction of applied force, e.g., centrifugal, gravitational, fluid magnetic, electric or fluid, force, that causes migration of detection reagent, is non-orthogonal or other than 90 degrees;

(h) providing a detection reagent under conditions sufficient for the formation of a complex, e.g., an immune complex, between said detection reagent and an anti-RBC antigen antibody in said sample,

(i) applying centrifugal force at said angle such that detection reagent that does not bind to said N^{th}, e.g., third, rbcm preparation migrates across said substrate,

wherein the presence or absence of detection reagent, e.g., in a preselected location, is correlated with the presence or absence of said anti-N^{th}, e.g., third, RBC antigen antibody in said sample,

thereby evaluating a sample for an anti N^{th}, e.g., third, RBC antigen_{—} antibody of pre-selected isotype, wherein in is equal to or greater than 3.

In certain embodiments, steps (a) and (g) are performed at least partially simultaneously. In other embodiments, steps (b) and (h) are performed at least partially simultaneously. In yet other embodiments, steps (c) and (i) described above are performed at least partially simultaneously.

In certain embodiments, the angle is optimized to allow one or more of:

(i) the migration of unbound detection reagent across said substrate;

(ii) the rapid migration of non-bound detection reagent across said portion, e.g., from said first positive readout region into said first negative readout region;

(iii) the migration of large aggregates, e.g., aggregates of 100, 1000, 10000, 100,000, 1,000,000, 10,000,000, 50,000,000 comparatively more rapid than the migration of smaller aggregates, e.g., 1, 2, or 4, or base units of detection reagent that are not detection reagent complexed into aggregates; or

(iv) the separation of non-bound detection reagent from detection reagent bound to an anti-RBC antibody, which anti-RBC antibody is bound to said first rbcm preparation (e.g., detection reagent in an immune complex with an said RBC antigen on said first rbcm preparation) on said substrate, e.g., substantially planar portion.

In other embodiments, the angle is between 2.5 and 10; between 10 and 35 (e.g., between 10 and 20; or between 20 and 35 degrees (e.g., typically, 10 degrees).

In another embodiment, the centrifugal acceleration is between 50-100, 100-300, or 300-1000 times normal gravitational acceleration. In certain embodiments, the centrifugal acceleration is applied for between 4-6, 2-4, and 0.5-2 minutes.

In yet other embodiment, the path of transit between a first positive readout region and a first negative readout region is between 8-50, 8-75, 8-100, 16-50, 16-75 or 16-100 microns.

In another embodiment, the method, or device, includes applying centrifugal force in two phases:

a first phase, having FN1, the force normal to the substrate, e.g., the substantially planar substrate, and FT1, the force tangential to said substrate, e.g., substantially planar substrate; and

a second phase, having FN2, the force normal to the substrate, e.g., substantially planar substrate, and FT2, the force tangential to said substrate, e.g., substantially planar substrate, wherein, said first phase occurs before said second phase, FN1 is greater than FN2, and FT2 is greater than FT1.

In yet other embodiment, the angle is constant during said first and second phase and the acceleration of the second phase differs from that of the first phase.

In one embodiment, the angle is constant during said first and second phase, and FN1 is greater than FN2, and FT1 is greater than FT2.

In another embodiment, the angle is constant during said first and second phase, and FN1 is less than FN2, and FT1 is less than FT2.

In one embodiments, the angle is dynamic and has a first average value during said first phase; and the angle has a second average value during said second phase, and

said first average value is less than said second average value, e.g., the second average value is at least 1.1, 2, 3, 4, 5, 10, or 100 times greater than said first average value. In one embodiment, the first average value is less than 10, 9, 8, 7, 6, 5, 4, 3, 2 or 1, e.g., said angle is 0. In other embodiments, the second average value is between 5-60, 10-40 and 15-25 degrees. In yet other embodiments, the first average value is less than 1, e.g., it is 0, and said second average value is 15-25 degrees.

In other embodiments, the average value is less than 1, e.g., it is 0, and 50-150, 5-125, 90-110, e.g., 100 g, are applied; and said second average value is 15-25 degrees and 100-300, 150-200, 175-225, e.g., 200 g, are applied.

In one embodiment, the second phase has a duration of between 10-270, 20-180, and 30-90 seconds. In other embodiments, the second phase has a duration of between 10-360, 20-240, and 30-120 seconds.

In yet another embodiment, the first phase and said second phase each has a duration of at least 10, 20 or 30 seconds but less than 360 seconds.

In another embodiment, the average centrifugal acceleration applied during said first phase is greater than that applied in said second phase, e.g., at least 2, 3, 4, 5, or 6 fold greater.

In other embodiments, the average centrifugal acceleration applied during said second phase is between, 100-3000, 150-2000 and 300-1,000 g.

In another embodiment, the average centrifugal acceleration applied during said first phase is between, 15-600, 30-400 and 50-200 g.

In other embodiments, in a regime where the number of cells is less than a mono layer, the length of time the cells move independently (i.e., the binding phase) is controlled. The longer the cells travel independently the greater the odds that a specific binding event will occur. Once the cells begin agglutinating and forming avalanches, specific binding, even if present, is typically not sufficiently large to stop the agglutins.

In certain embodiments, the duration of the ‘binding phase’ is governed by cell concentration, cell velocity, and non-specific binding. Hence, the centrifuge angle and acceleration effect cell velocity. An expression for the cell velocity follows:

$${v}_{cell} \approx αβ {r}^{2} sin ( θ )$$

$$α = \frac{{ρ}_{cell} - {ρ}_{so ln}}{η}$$

$$β = \frac{{V}^{2}}{R}$$

V=velocity of centrifuge
R=radius arm of centrifuge
r=radius of cell
η=viscosity of solution
ρ_{cell}=density of cell
ρ_{so ln}=density of solution
θ=angle of incline
   Alpha, in the above expression, is made up of physical parameters (densities, viscosity). Beta is proportional to the centrifugal acceleration (i.e., g's). sin(theta) is the angle of incline.

At the same time, the test time must be short. Therefore, the cell velocity (and indicator cell agglutination rate) needs to be sufficiently large that should specific binding be absent, the indicators can run into each other and form the avalanches.

In a regime where the concentration of indicator cells is larger than a monolayer, the behavior of a membrane of indicator cells is analyzed. In this regime, the indicator cell concentration is optimized in concert with centrifuge angle, acceleration, and time to produce a system in which a three-dimensional network of indicator cells is forced across a biologically active surface.

In other embodiments, said centrifugal acceleration and said angle result in an acceleration acting on indicator moieties in the direction normal to said substrate, e.g., substantially planar substrate, in the range of 15-1000, 50-500 and 35-800 g's and an acceleration acting on indicator moieties in the direction tangential to said substrate, e.g., substantially planar substrate, in the range of 0-500, 25-130 and 10-400 g's.

In yet other embodiments, said centrifugal acceleration and said angle result in a ratio between the tangential acceleration and the normal acceleration to said substrate, e.g., substantially planar substrate, in the range of 0-0.5, 0.1-0.25 and 0.05-0.4.

In one embodiment, said angle is between 10 and 25 degrees; said centrifugal acceleration is between 50-1000 g; and said centrifugal acceleration is applied for between 0.5-4 minutes

In one embodiment, the negative readout region is located on said substrate, e.g., substantially planar substrate. In other embodiments, the negative readout region is not located on said substrate, e.g., substantially planar substrate.

In yet other embodiments, the method or device further includes detecting the presence of said detection reagent in a first positive readout region, e.g., on said substrate, e.g., substantially planar substrate.

In other embodiments, the method or device further includes comparing a value for the amount of detection reagent present in said first positive readout region with a pre-selected criterion, and if said value meets said pre-selected criterion classifying said sample, e.g., as positive.

In other embodiments, the method or device further includes detecting the presence of said detection reagent in said first negative region.

In other embodiments, the method or device further includes comparing a value for the amount of detection reagent present in said first negative readout region with a pre-selected criterion, and if said value meets said pre-selected criterion classifying said sample, e.g., as negative.

In certain embodiments, the detection reagent includes an IgG-specific binding moiety as described herein. In one embodiment, the detection reagent includes a binding moiety having specificity for an isotype-common site on said anti-blood-type-antigen antibody of a pre-selected isotype, e.g., IgG.

### Detection Reagents

In other embodiments, the method or device, further includes forming detection reagent complexes, e.g., aggregates between detection moieties, e.g. between the indicator moieties of detection moieties. The method includes contacting a detection reagent with a detection moiety complexing agent that promotes the formation of a detection reagent complex between base units of detection reagent. For example, a human red blood cell sensitized with a monoclonal anti-D, e.g., Alba Z441.

In one embodiment, the detection reagent comprises a moiety that promotes detection reagent complexing between base units of detection reagent.

In other embodiments, the level of detection reagent complexing between detection moieties is sufficient that it increases the signal from detection reagent in a positive readout region, a negative readout region, or both.

In other embodiments, a base unit of detection reagent, e.g., a base unit of detection reagent bound to a rbcm preparation, acts as a nucleation site for growth by detection reagent complexation with another base unit of detection reagent.

In yet other embodiments, a base unit of detection reagent, e.g., a base unit of detection reagent bound to a rbcm preparation, is detection reagent complexed to a second base unit of detection reagent.

In other embodiments, a base unit of detection reagent, e.g., a base unit of detection reagent is bound to a rbcm preparation, is detection reagent complexed to a second unbound base unit of detection reagent.

In yet other embodiments, a base unit of detection reagent, e.g., a first base unit of detection reagent bound to a rbcm preparation, enhances the ability of a second base unit of detection reagent to bind to said rbcm preparation, e.g., by detection reagent complexing between said first and second base unit.

In other embodiments, the detection reagent complexing is sufficient such that the time required for a non-specifically bound detection reagent to migrate into a negative readout region is less than the time required in the absence of detection reagent complexation.

In other embodiments, the time with detection reagent complexing is less than 90, 80, 70, 60, 50, 40, 30, 20, or 10% of the time in the absence of detection reagent complexing.

In another embodiment, the detection reagent complexing is sufficient such that non-specific binding of detection reagent to rbcm is less than that in the absence of detection reagent complexing.

In other embodiments, the non-specific binding with detection reagent complexing is less than 90, 80, 70, 60, 50, 40, 30, 20, or 10% in the absence of detection reagent complexing.

In other embodiments, the detection reagent further includes an indicator moiety, e.g., a cell, e.g., a red blood cell.

In one embodiment, the detection reagent includes a deformable component, e.g., a cell, e.g., a red blood cell, or an entity which is similarly deformable.

In one embodiment, the method, or device, includes using a detection reagent comprising a deformable component, e.g., a cell, e.g., a red blood cell, or an entity which is similarly deformable; applying a normal force, e.g., centrifugation under condition wherein said centrifugal force is normal or within 30 degrees of normal, to said substantially planar substrate; and applying a tangential force, wherein said normal force is greater than said tangential force, e.g., at least 4, 3, 2 fold greater.

In one embodiment, a specifically bound component, if compliant under applied forces, can be flattened (inclusive of stretching, flattening, or other changes in morphology) upon application of a force.

### Exemplary Configurations and Applied Forces for Solid-Phase Capturing Methods

This aspect of the invention described below is exemplified in the context of solid phase antibody screening antibody screening. However, same techniques can be applied to any system in which a particle is specifically bound to a surface (for instance, antibody identification and immunodiagnostics).

In conventional solid-phase antibody screening, a substrate is coated with red blood cell membranes of known antigen content. Plasma is incubated over this surface and then removed via washing. Antibodies with specificity for antigens existing on the substrate blood cells will remain. At this point, some form of anti-human globulin (AHG) attached to an indicator (e.g., fluorescent, cellular, colloidal) is added to the system. The AHG will bind to human immune globulin (if present) and the indicator provides means of visualizing binding.

Conventional assays rely on indicators generated by first coating a red blood cell with an IgG antibody specific for an antigen present on the indicator (for instance, the D antigen). The cells are washed and then anti-IgG antibody is added to generate an anti-IgG coated particle. The anti-IgG antibody is added at a sufficient concentration such that virtually all of the IgG existing on the indicator is coated with anti-IgG antibody. This renders the indicators stable against agglutination.

Once the indicators are added to the system, a force is typically applied in order to produce a more clear measurement. For instance, if a round or ‘U’ bottom well is used in conjunction with a swinging bucket centrifuge, unbound cells will be forced to the bottommost portion of the well. If a specific bond is formed between an indicator and the solid phase, and the centrifugal force acting on the indicator is less than the binding force existing between the indicator and solid-phase, the indicator will remain (to some degree) dispersed across the solid-phase. If binding is insufficient to counter the centrifugal force, the indicator will migrate to the lowest position in the well. Hence, if a ‘button’ of indicator particles is present at the bottommost portion of the well, a negative result can be inferred. If such a ‘button’ is absent (or diminished) a positive result can be inferred.

Conventional assays that utilize round bottom or non-planar geometries do not produce optimal sensitivity. To begin, the geometry of the well and the configuration of the centrifuge control the magnitude and the direction of the forces applied to the indicator. For instance, if a flat-bottom surface is used in conjunction with a swinging bucket centrifuge, an indicator cell will only experience a normal force that drives it to the surface and no differentiation between bound an unbound can be made. Alternatively, if an inclined plane is introduced such that the plate resides at a non-orthogonal angle relative to the radial direction, a tangential force will be applied to the indicator. The magnitude of the relevant forces in the directions tangent and normal to the inclined plane are given by the product of the centrifugal force (Fc) times the sin (tangential) or cos (normal) of the inclined plane angle. These configurations are represented in schematic form in FIGS. 13A-13C. FIG. 13A is a schematic top plane view of a swinging bucket centrifuge operating in a clockwise direction. The arrow indicates the axis of rotation. FIGS. 13B-13C is a free body diagram representing the normal and tangential forces acting on a cell (Fc is a centrifugal force). In this context, if no other limiting factors (for instance, non-specific binding) are significant in magnitude, a small theta combined with a minimal centrifugal force is ideal. Such a situation would first drive and then push an indicator into close proximity with the surface of interest. A small lateral force would push the indicator across the surface at a rate dependant on the particle size, solute viscosity, and centrifugal force. In this regime (low angle and low centrifugal force) the indicator would slowly travel across the solid-phase and probe potential binding sites. The low speed (as compared to those induced by high centrifugal forces or angles) increases the interaction time existing between an indicator and potential binding sites. Ultimately, this increase in time should lead to a higher percentage of indicators bound to the surface. Furthermore, a large normal component acting on the indicator can increase the area of interaction between a deformable particle and the solid-phase. This should further increase binding between an indicator and the solid phase.

Moreover, the low centrifugal force and low angle produce less tension on a specific bond once it is formed. This should lead to a greater percentage of indicators bound to the surface once a measurement is conducted.

Non-planar geometries are now compared to the proposed optimal geometry. A round bottom or ‘U’ bottom well of typical design produces low angle inclines only at the very bottom of the well. Hence, most of the well area produces relatively large tangential forces and relatively weak normal forces. In addition, the bottom portion of the well that may produce optimal binding conditions is typically occupied by truly unbound cells and therefore is inaccessible to measurement. This is quite non-ideal and can be significantly improved upon by utilizing a planar geometry combined with a small incline.

In addition to the geometry and configuration of the centrifuge, the preparation of the indicator can strongly influence test performance. Conventional indicators are rendered incapable of agglutination via saturation with anti-IgG. However, it is advantageous to allow the cells to adhere to one another. For instance, if an indicator cell binds specifically to the surface and another unbound indicator cell contacts this cell, it may become part of the complex that is specifically bound to the substrate. Essentially the first specifically bound particle acts as a nucleation site for growth and therefore the overall effect can be amplification of the number of indicators adhered to the surface. Such an effect is useful on both macroscopic (i.e., reading by ‘eye’) and microscopic levels.

In addition, cooperative effects may occur in such a system. If a free indicator cell is captured by a solid-phase bound cell, the rate at which new solid-phase/indicator bonds form may increase due to the fact that the originally unbound indicator is now localized to a specific region.

Thus, the binding of individual indicator cells can encourage the binding of other individual indicator cells. This indicator preparation also has the effect wherein unbound indicators can encourage other indicators to not bind. This occurs because two unbound indicator cells which are traveling along the surface may naturally travel at speeds which differ from their average speed, thereby allowing unbound indicators to bump into each other and aggregate. Such coupled indicator cells, or aggregates, may travel more quickly and may more readily overcome interactions with the surface. Thus, aggregates have a tendency to speed up and collect further indicator cells. In this way, there is collective behavior which tends to amplify the presence or absence or binding to the surface and to make such a result readily apparent at shorter forcing times and with shorter incubation steps.

This change in preparation can also significantly reduce both the time required to run the assay and the magnitude of the signal presented by negative samples. To begin, the centrifugal force acting on a cell is proportional to mv̂2/r where m is the effective mass of the particle, v is the velocity of rotation, and r is the radius arm. This force is opposed by the viscous drag induced on the cell by the solution. This force is proportional to 6πηrv where η is the viscosity of the solution, r is the cell radius, and v is the cell velocity. If the indicator cells are capable of agglutinating, the mass of such complexes will increase as the number of cells residing in a complex increases. This change in mass produces a larger force which, if the cells are unbound, produces a larger complex velocity. As the length of time the centrifugal force must be applied is set by the unbound cell velocity and distance needed to travel, this change in velocity allows the test to be conducted in less time.

Furthermore, these complexes, if controlled properly, can effectively reduce the magnitude of non-specific binding. To begin, the rate at which the indicators agglutinate is set by: the density of labeling IgG, the concentration and binding characteristics of the AHG, the concentration and volume of the indicator cells, the rate of centrifugation, the angle of centrifugation, and the interaction with the surface. As an example, if too few indicators reside on the solid phase, the cells will only occasionally be in close enough proximity to agglutinate and the rate of agglutination will be low. If a tangential force is applied to these cells they will effectively travel independently. Alternatively, if too many of the indicators reside on the surface, they will rapidly agglutinate. If a tangential force is applied to these complexes, they will travel at a rate greater than single cells. If these complexes reach sufficient size, they essentially form an avalanche of indicators that rapidly moves across the substrate and may scavenge both bound and unbound cells. Hence, if this avalanche is triggered properly, it can effectively reduce the magnitude of non-specific binding.

Enabling the particles to adhere to one another can be achieved by a number of techniques. For instance, simply adding the anti-IgG at an appropriate concentration to the IgG-coated indicators immediately before conducting a solid-phase test will produce such behavior.

The distinction of enabling the particles to adhere to one another can be vital to these techniques. In one regime, the concentration of indicator particles is less than that required to form a monolayer of particles. In this case, the particles spend a non-trivial portion of time as independent objects and the aforementioned discussion applies. In another regime, the concentration of indicator particles is significantly higher (3-10X) than that required to form a monolayer. In this case, the motion of individual cells is no longer relevant as once the cells are added to the well and centrifugation has started, they spontaneously self-assemble into a three dimensional membrane. In this situation, the membrane of indicator cells is forced across the coated well surface and its behavior is used to deduce the test result. If IgG is specifically bound to the test surface, the membrane of indicator cells binds to the IgG and motion is suppressed. If IgG is absent, the membrane or portions of the membrane can travel across the surface and produce a portion of the well-bottom that largely lacks indicator cells.

In these embodiments, the indicator is a red cell coated with IgG. However, coated microparticles, vesicles, and other cells could also be used as indicators as long as they are prepared in such a way that enables complexation once added to the test well. In addition, the acceleration applied to such indicators should be adjusted to counter any change in indicator effective mass, characteristic dimension, solid-phase binding rate, and complexation rate.

In certain embodiments, maximization of specific binding and reduction of test time can be accomplished by one or more of:

(i) applying a high normal force/low tangential force and proper indicator/anti-human globulin preparation can increase specific binding; or

(ii) controlling the indicator cell concentration/agglutination rate and high tangential force can decrease test time.

The following parameters can be considered to optimize antibody screening test quality:

1. Specific binding/non-specific binding ratio: A minimum signal/noise threshold is necessary to properly trigger this effect.

2. Centrifuge angle and rate:

Magnitude of normal force [rate of specific binding is enhanced by larger normal force—i.e., shallower angle of incline]

Magnitude of tangential force [rate of specific binding is enhanced by low tangential force, rate of avalanche formation is enhanced by large tangential force—i.e., larger angle of incline]

3. Anti-human globulin concentration

Effects rate of specific binding [the AHG concentration should be balanced such that binding sites are neither starved nor quenched]

Effects rate of avalanche formation [the AHG concentration should be balanced such that binding sites are neither starved nor quenched]

4. Indicator cell concentration/volume

Sub-monolayer concentrations produce a system influenced by individual cell effects. Concentrations exceeding a monolayer produce a system driven by bulk effects linked to the motion of a membrane driven across a surface.

Effects the magnitude of the motion of the indicator cell membrane Effects rate of avalanche formation [the greater the cell concentration the higher the odds that two indicator cells will see each other and trigger the avalanche process]

5. Indicator cell preparation

Effects rate of specific binding [the number of IgG sites on the indicator cell should, ideally, be large]

Effects rate of avalanche formation [the number of IgG sites on the indicator should be large—this is a minor feature]

6. Ionic strength

Effects rate of specific binding [low ionic strengths typically enhance binding]

7. Centrifugation time

Effects degree of Avalanche formation (i.e., size of agglutins and position), and, to some extent, the degree of specific binding (i.e., if you spin for too long specific bonds will break)

8. Multi-stage and/or angle of incline

As there are two distinct phases of this portion of the test (optimal conditions for specific binding followed by optimal conditions for triggering the avalanche process) varying the centrifugal acceleration or angle of incline produces overall optimal performance.

### Exemplary Solid Phase Configurations

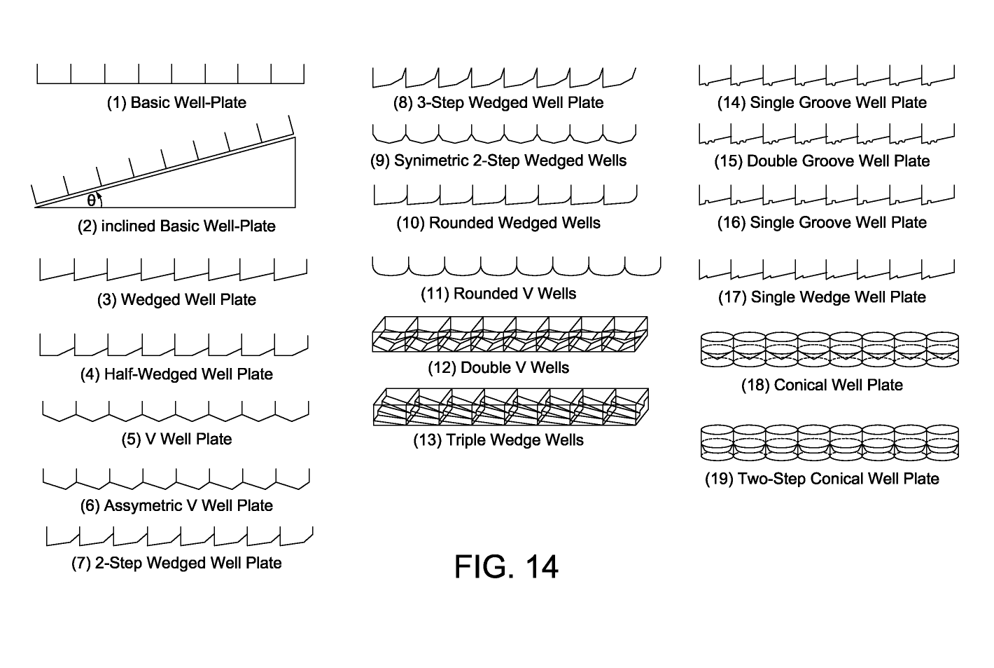

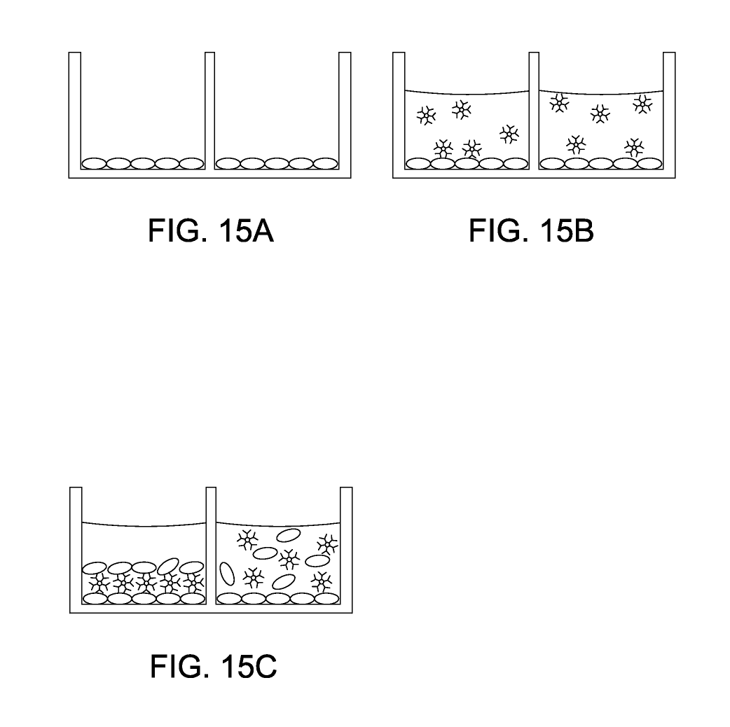

In other embodiments, the methods and devices of the invention can be carried out using one or more of the exemplary well plate configurations depicted in FIG. 14. Such plate geometries are believed to create the right balance of normal force and tangential force; to have different normal forces and tangential forces in different wells; to eliminate the radius arm problem and tilted plate problem (i.e., ensure two wells have identical force profiles even though they are in different locations, such as distance from the central rotational axis, when being centrifuged); to do different tests in different wells at the same time; to accelerate (nucleate) the avalanche effect; to do multiple tests within a single well on a single sample; or to generally improve the imaging and/or discernment of positive vs. negative samples. (Note that in many cases, it may make imaging more difficult to detect.)

The 19 well plate geometries shown in FIG. 14 are described as follows:

(1) Basic commercial well plate.

(2) Basic commercial well-plate inclined at angle theta to produce correct ratio of F_normal to F_tangent.

(3) Custom well plate designed to have angle introduced into well bottom (manufactured into plate) rather than to be centrifuged at an angle. Note: Each well could have a different angle if required, such as for different tests. This would function similarly to (2) which we are doing now. It requires a custom well plate and a suitable reader, but eliminates the complexity of centrifuging with an angle. It also allows for different wells to be under different conditions, which is not presently possible with (2).

(4) This plate can be operated in either of two ways: (a) It can be placed into an angled centrifuge to have two angles in a given well. This may have the benefit of having a region of higher angle where the RBCs more quickly move and form aggregates of a certain size, if free. This may speed up the aggregate formation and avalanche for negative samples. The remainder of the well is at a normal, lower angle. (b) It can be centrifuged without an angle so as to have the clusters stop at a place away from the wall for easier reading.

(5) V Well Plate: This can be centrifuged without an angle. It has the benefit that unbound aggregates go to the centerline which is easier to see and measure.

(6) Asymmetric V Well Plate: As with (5) above, except the asymmetry can be used to either measure the response at two angles or to allow one to perform the test using the shallow slope side, giving more distance and area to work with.

(7) 2-Step-Wedged Well Plate: This is similar to (4) above but doesn't need to be centrifuged at an angle to obtain the two non-zero angle case. The idea is that cells first sprinkle down uniformly. Then cells quickly slide down the steeper slope and form some smallish aggregates (if they do not bind to surface). When they reach the lower slope, for negative samples, the aggregates start with significant size meaning that negative samples will have a much stronger avalanche, faster. If it is positive, the avalanche will not be initiated. Thus, this is meant as a means to nucleate and speed up the avalanching process for negative samples.

(8) 3-Step-Wedged Well Plate: This is like the 2-step-wedged well plate, except it has another wedge. The steepest wedge quickly nucleates small clusters. The next wedge allows these to grow to a certain size if the sample is negative. The main wedge allows them to grow to the “unstable” size if still no binding occurs.

(9) Asymmetric 2-Step Wedge: This is like (8) except it is symmetric so that the negative region is more easily read, since it is in the centerline.

(10) Rounded Wedged Wells Plate: The rounding at one edge achieves something equivalent to the 3-step wedged well plate.

(11) This combines the rounded wedged well plate simplicity with the convenience of symmetry so that cells collect in the centerline for negative samples, for easy and accurate reading. (Note that these rounded wells are quite distinct from what some others use at present, since most of the well is still essentially planar and at a prescribed (low) range of angles. The cells that collect do not obscure the important angle region.).

(12) Double V Well Plate: This allows each sample to simultaneously be tested under two angles. This may be used for quantization, among other things.

(13) Triple Wedge Well Plate: As with the double-V well plate, this allows one to perform multiple tests on the same blood in one well. It can be used to get data at different angles, such as for quantization.

(14) Single Groove Well Plate: This has a single groove in the surface which catches unbound cells or aggregates (perhaps of a certain size or less). This makes it easier to read negative samples.

(15) Double Groove Well Plate: This has two grooves (or more) of the same or differing sizes or shapes. This can help discriminate a low level of aggregation and a higher level of aggregation.

(16) Single Hump Well Plate: This is functionally fairly similar to the Single Groove Well Plate.

(17) Single Wedge-Hump Well Plate: This is functionally pretty similar to the Single Hump Well Plate.

(18) Conical Well Plate: This is like a V Well Plate but has azimuthal symmetry.

(19) Two-Step Conical Well Plate: This is like a Symmetric Two-Step V Well Plate (#9) but has azimuthal symmetry.

### Exemplary Substrate Configurations

Methods and devices of the invention use substrates, e.g., substantially planar substrates, on which a variety of entities are disposed including, for example, capture reagents, antibodies, and rbcm preparations. In certain embodiments, cells, aggregates, detection reagent or aggregated detection reagent, migrate across said surfaces. The behavior of these entities can be important in the performance of a test. By way of example, the ability of an entity to migrate across a surface, to encounter other entities not complexed with the surface, associate with those other entities, e.g., to form aggregates, and to form a detectable formation, can be important. Numerous approaches for optimizing one or more of these behaviors are disclosed herein.

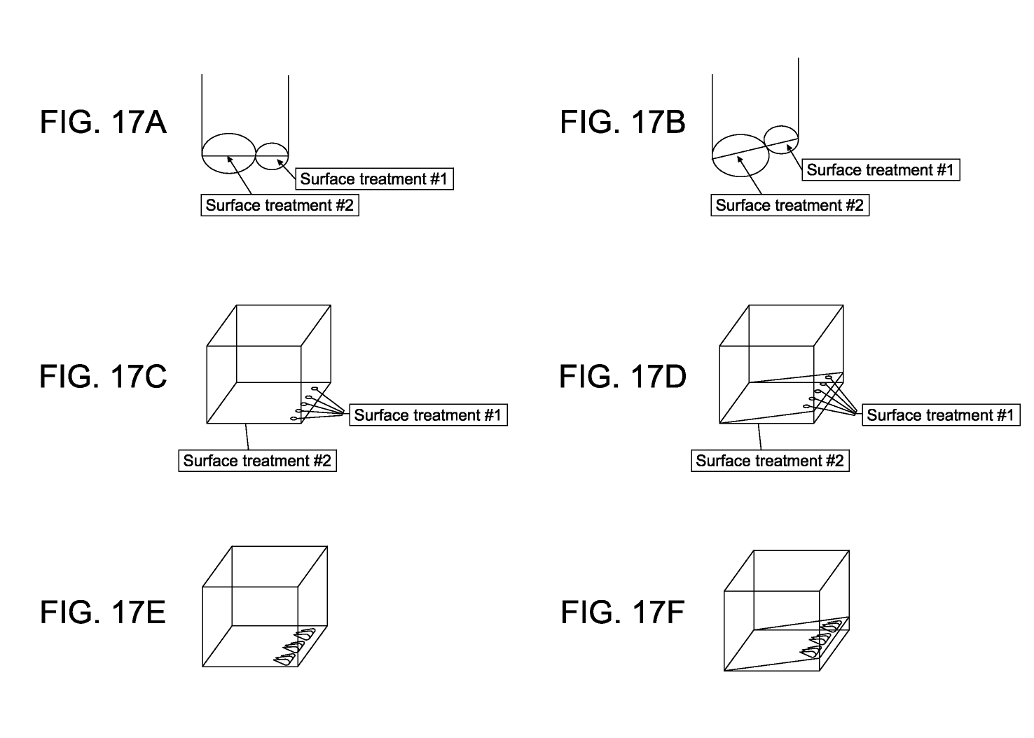

FIGS. 17A-17F illustrate a number of exemplary substrate configurations described herein as follows.

Substrate (A) shows a surface with two adjacent surface regions with different surface treatments. For example, surface treatment #1 can be a treatment (or lack of a treatment) such that probe cells do not adhere to the surface, and surface treatment #2 can be a treatment such that binding occurs for positive samples. Surface region #1 could be optimized such that aggregates of a desired size generally form before reaching region #2.

Substrate (B) depicts a surface similar to A above, except with angled bottom.

Substrate (C) depicts a surface with multiple surface regions wherein multiple surfaces of a desired size and shape and location are treated with surface treatment #1; a treatment or lack of a treatment such that binding does not occur) are adjacent to a surface with treatment #2 (e.g., a surface where binding occurs only if the sample is positive, or only if the sample is negative). The former regions may be located in a position and of a size and shape so as to cause aggregates of a given size (range) to form before reaching the surface region with treatment #2.

Substrate (D) depicts a surface similar to Substrate (C) except on inclined plane.

Substrate (E) depicts a surface with one or more regions which have steeper angles which are continuous (and “upstream”) of a region (which can be a substantially planar) which is of a given angle. These regions may have a “funnel” characteristic, able to collect together those objects which move along the surface with the steeper angle. Such features may be used to collect together a number of particles which are close in number to a desired number, such as to create (nucleate) aggregates of a desired size.

Substrate (F) depicts a surface similar to D except on inclined plane.

With a simple planar surface (which can be a substantially planar), a negative sample may reveal that it is negative through the random process of particles contacting each other as they slide down the surface, forming aggregates which move more quickly and gather additional particles. Thus, an avalanche occurs. This process is random in nature, and thus may require large surfaces or be subject to random or unusual events. To mitigate this effect, the nucleation of aggregates can be controlled through the design of the surface. The simplest way of doing so is to put an “acceleration” region above a substantially planar surface. This acceleration region could be a substantially planar surface at a steeper angle, it could be a non-planar surface such as a curved surface, or it could be a region with different surface properties (see FIGS. 17A-17B). To control the formation of aggregates even more precisely, the surface may be patterned with a treatment such that regions of a desired size, shape, and location may be placed to encourage the formation of aggregates of an approximate size (see FIGS. 17C-17D). Another way to create aggregates of an approximate size is to create features (eg. geometric features) which may gather a certain approximate number of particles and encourage them to form an aggregate (eg. a “funnel”-type surface region which gathers cells as they fall and collects them into one or more aggregates) (see FIGS. 17E-17F).

Any of the methods or devices described herein can incorporate one or more of the following features:

In one embodiment, the substantially planar substrate is adjacent a region having a steeper angles, e.g., an angle which minimizes binding of cells, aggregates, detection reagent or aggregated detection reagent.

In another embodiment, the substantially planar substrate is adjacent a region having a different surface treatment.

In an embodiment, the substantially planar substrate is adjacent a region having is configured so as to enhance nucleation, e.g., a region which increases the concentration of particles in the direction of migration.

In an embodiment, the substantially planar substrate is adjacent a region having which concentrations migrating particles, e.g., a region configured as a funnel.

In an embodiment, the substantially planar substrate is adjacent a region having a feature which improves the detectability of negative samples, e.g., a feature which impedes the passage, captures, or concentrates migrating entities, e.g., cells, aggregates, detection reagent or aggregated detection reagent. By way of example, the region can comprise a depression, e.g., a pit or groove, or an elevation, e.g., a bump or ridge, or a discontinuity or interface, e.g., between two regions.

In an embodiment the substrate comprises an interface between two surface regions wherein migrating cells, aggregates, detection reagent or aggregated detection reagent, can collect, and the presence, absence, or quantity of cells in this region can inform a test result.

In an embodiment, a substrate comprises a plurality of surfaces, e.g., planar or substantially planar in sufficient proximity allow performance of a plurality of tests, e.g., two tests, with different properties, e.g., sensitivities, e.g., to quantitate the test result.

In one embodiment, a substantially planar surface region is azimuthally symmetric.

In another embodiment, a carrier, e.g., a plate, having a plurality of substantially planar surface regions, disposed at more than 1 different angle, so that tests can be performed at different conditions, such as for doing two different tests with different parameters at the same time on the same plate.

### Detection of Aggregate Related Features

Migration of detection reagent units across a substrate, e.g., substantially planar substrate, can result in a detectable event. E.g., migration of a detection reagent unit across a rbcm preparation on such a substrate can result in aggregation of the migrating detection reagent unit with other detection reagent units, e.g., more slowly migrating detection reagent subunits, forming an aggregate. The aggregate is an example of, or can serve as the basis of a detectable event. E.g., the existence, number, or location of aggregates can be a detectable event. Aggregate formation and migration can be accompanied by regions of substrate that differ from regions having no aggregate formation and migration. While not wishing to be bound by theory it is believed that the area of the path taken by an aggregate will be depleted, as compared to an otherwise similar are of the substrate, of unbound detection reagent. The area of the path can be distinguished, e.g., from a reference, e.g., from an otherwise similar area than has not been depleted by aggregate formation. The depleted path is an example of, or can serve as the basis of, a detectable event. E.g., the existence, number, or location of depleted paths can be a detectable event. E.g., one can compare a first region or field with a second region or field for differences in a detectable event. In an embodiment a preselected value for a parameter related to such a detectable event, e.g., the presence, level, distribution, or location of one or more detectable events, e.g., aggregates or depleted regions, is indicative of the presence or absence of an analyte. E.g., the presence of an aggregate of depleted area or path can be indicative of the absence of analyte. Detection devices, e.g., scanners and associated analytic software and readout devices can be configured for evaluating detectable events.

Devices and Methods for Separating a Plasma Sample from Whole Blood

Plasma samples can be obtained by methods known in the art. In one embodiment, the plasma sample can be separated from a whole blood sample using a rotor described in U.S. Ser. No. 61/438,571, entitled “Centrifuge Rotor for Separation and Processing of Complex Fluids” filed on Feb. 1, 2011, incorporated herein by reference. In certain embodiments, the rotor is used in a centrifuge system. The rotor includes a housing fabricated from a lightweight, transparent or translucent material, such as plastic. In one embodiment, the housing is generally disc-shaped, and includes a central opening that is configured to be secured to the centrifuge. The rotor may be further configured with alignment features that enable the rotor to be registered in a specific orientation with respect to the centrifuge system for indexing the position of the rotor. The rotor is configured with one or more chambers, e.g., twelve, each chamber receiving a sample of whole blood, or some other type of biological fluid requiring separation. The arrangement is such that the centrifuge spins the rotor to separate plasma from red blood cells contained within the whole blood. In a certain embodiment, each chamber includes a first chamber portion having an opening that serves as an inlet/outlet opening for the chamber and a second chamber portion in fluid communication with the first chamber portion. The first chamber portion has a port formed therein, with the second chamber portion being in fluid communication with the port of the first chamber portion. In a particular embodiment, the second chamber portion has a fill line disposed generally below the port of the first chamber portion. This construction ensures that when a centrifuge operation takes place, red blood cells are retained in the second chamber portion and plasma is retained in the first chamber portion. Thus, the red blood cells are retained in the second chamber portion, both during and after the relaxation and removal of the plasma within the first chamber portion. In another embodiment, the first chamber portion and the second chamber portion extend along a radial axis of the rotor. The first chamber portion and the second chamber portion are configured so that when a centrifuge operation takes place, a first biological fluid type (e.g., plasma) is retained in the first chamber portion and a second biological fluid type (e.g., red blood cells) is retained in the second chamber portion. The second chamber portion has a capacity greater than an amount of the second biological fluid type retained in the second chamber portion. In yet another embodiment, a channel provides fluid communication between the first chamber portion and the second chamber portion. The channel is configured so that when a centrifuge operation takes place, a first biological fluid type is retained in the first chamber portion and a second biological fluid type is retained in the second chamber portion. In another embodiment, the rotor is configured to receive a plurality of disposable containers, e.g., twelve, which are designed to receive complex fluids for processing.

Other embodiments or features of the invention include one or more of the following.

1. A method of evaluating a sample for a red blood cell antigen, comprising:

(a) contacting a red blood cell antigen antibody disposed on a surface with a sample containing one or more red blood cells, under conditions sufficient for the formation of a complex between said red blood cell (RBC) antigen antibody, and a red blood cell in said sample to occur, wherein said red blood cell comprises the red blood cell antigen (referred to herein as “complexed cells”);

(b) separating the complexed cells by causing differential migration of red blood cells not complexed with said red blood cell antigen binding antibody (“uncomplexed cells”), relative to the complexed cells, across said substrate,

wherein an increase or a decrease in the amount of complexed and/or uncomplexed red blood cells is correlated with the presence or absence of binding between said red blood cell (RBC) antigen antibody and said sample,
thereby evaluating a sample for a red blood type antigen.

2. The method of claim 1, wherein the red blood cell antigen is a blood-type antigen chosen from an A, B, AB, or a D antigen.

3. The method of claim 1-2, wherein the red blood cell antigen is chosen from one, two, three, four, five, six, seven, eight, nine, ten, eleven, twelve, or more, or all of:

a Rhesus antigen chosen from one or more or all of D, C, c, E, or e;

a MNS antigen chosen from one or more or all of M, N, S, or s;

a Kidd antigen chosen one or both of Jk^{a} or Jk^{b};

a Duffy antigen chosen one or both of Fy^{a} or Fy^{b};

a Kell antigen chosen one or both of K or k;

a Lewis antigen chosen one or both of Le^{a} or Le^{b}; or

a P antigen.

4. The method of claim 1-3, wherein said red blood cell antigen binding antibody is disposed on a surface, and the angle between said surface, and the direction of an applied force, that causes migration of the detection reagent, is non-orthogonal or other than 90 degrees, wherein the applied force is chosen from one or more of a centrifugal, a gravitational, a fluid magnetic, an electric or a fluid force.

5. The method of claim 1-4, wherein said method includes applying centrifugal force in at least two phases:

a first phase having FN1, the force normal to the surface, and FT1, the force tangential to said surface or substrate, and

a second phase having FN2, the force normal to the surface, and FT2, the force tangential to said surface, wherein said first phase occurs before said second phase.

6. The method of claim 5, wherein said angle is chosen as a constant angle during said first and second phase, and FN1 is greater than FN2, and FT1 is greater than FT2, or FN1 is less than FN2 and FT1 is less than FT2.

7. The method of claim 1, wherein the presence or absence of the anti-RBC antigen antibody in the sample is indicated by a value of a parameter corresponding to the behavior of, or related to the positional distribution of, the detection reagent chosen from one or more of the amount of the detection reagent; an increased or decreased presence of the detection reagent; the pattern of coverage of the surface by the detection reagent; the amount of coverage of the surface by the detection reagent; the distribution of the detection reagent on a surface; or the strength of adherence of the detection reagent bound to the binding agent on the surface.

8. A method of providing a substrate having red blood cells, or a red blood cell membrane preparation, bound thereto comprising:

providing a substrate capable of binding red blood cells;

contacting said substrate with a solution of red blood cells to form a solution-contacted-substrate;

centrifuging said solution-contacted-substrate for a time sufficient to cause red blood cells in said solution to settle onto said substrate;

optionally, washing said substrate to remove unbound red blood cells;

optionally, lysing red blood cells bound to said substrate to provide a rbcm preparation bound to said substrate;

thereby providing a substrate having red blood cells, or a rbcm preparation, bound thereto,

wherein, optionally, said substrate having red blood cells, or a rbcm preparation, bound thereto, has one of the following properties:
said centrifugation is sufficient in force and duration such that, if red blood cells are dispersed on the substrate having red blood cells, or rbcm preparation, bound thereto, less than 10, 5, or 1% of the dispersed red blood cells are non-specifically bound, e.g., as determined by optical trap measurement;
said centrifugation is sufficient in force and duration such that if red blood cells are dispersed on the substrate having red blood cells, or rbcm preparation, bound thereto, the non-specific binding of red blood cells to said substrate having red blood cells, or rbcm preparation, bound thereto, is less than 50, 40, 30, 20, 10, 1.0, 0.1, or 0.01% of the non-specific binding of red blood cells to a substrate having red blood cells, or rbcm preparation, bound thereto, prepared in a similar manner except that the red blood cells are deposited on the substrate by gravitational settling as opposed to centrifugation.

9. A method of evaluating a sample from a subject, for an anti-RBC-antigen antibody of G isotype, comprising:

(a) contacting a first mimic-optimized red blood cell membrane preparation (a mo-rbcm preparation) comprising a first RBC antigen with sample from said subject, under conditions sufficient for the formation of an immune complex between said first RBC antigen and anti-first-RBC antigen antibody in said sample; and

(b) providing a detection reagent under conditions sufficient for the formation of a complex, e.g., an immune complex, between said detection reagent and an IgG antibody in said sample, said detection reagent comprising an IgG binding moiety,

wherein, the behavior, or positional distribution, of said detection reagent e.g., in a preselected location, is correlated with the presence or absence of said anti-RBC antigen antibody in said sample,

thereby evaluating a sample for an anti-RBC antigen antibody of G isotype.

10. The method of claim 9, wherein said mimic optimized rbcm preparation is a rbcm preparation that has been contacted with a proteolytic enzyme.

11. The method of claim 10, wherein the enzyme is an immunoglobulin G-degrading enzyme.

12. A substrate having red blood cells, or a rbcm preparation, or a mimic optimized-rbcm preparation, bound thereto, wherein if red blood cells are dispersed on the substrate having red blood cells, or rbcm preparation, bound thereto, less than 10, 5, or 1% of the dispersed red blood cells are non-specifically bound.

13. A substrate having red blood cells, or a rbcm preparation, or a mimic optimized-rbcm preparation, bound thereto, wherein if red blood cells are dispersed on the substrate having red blood cells, or rbcm preparation, bound thereto, the non-specific binding of dispersed red blood cells to said substrate having red blood cells, or a rbcm preparation, bound thereto, is less than 50, 40, 30, 20, 10, 1.0, 0.1, or 0.01% of the non-specific binding of dispersed red blood cells to a reference substrate, e.g., a substrate having red blood cells, or rbcm preparation, bound thereto, prepared in a similar manner except that the red blood cells are deposited on the substrate by gravitational settling as opposed to centrifugation.

14. A device for evaluating a sample from a subject, for a an anti-RBC antigen antibody, comprising:

a channel comprising
a) a substrate having red blood cells, or a rbcm preparation, e.g., a mo-rbcm preparation, bound thereto, wherein
   if red blood cells are dispersed on the substrate having red blood cells, or rbcm preparation, bound thereto, less than 10, 5, or 1% of the dispersed red blood cells are non-specifically bound, e.g., as determined by optical trap measurement; or
   if red blood cells are dispersed on the substrate having red blood cells, or rbcm preparation, bound thereto, the non-specific binding of dispersed red blood cells to said substrate having red blood cells, or a rbcm preparation, bound thereto, is less than 50, 40, 30, 20, 10, 1.0, 0.1, or 0.01% of the non-specific binding of dispersed red blood cells to a reference substrate, e.g., a substrate having red blood cells, or rbcm preparation, bound thereto, prepared in a similar manner except that the red blood cells are deposited on the substrate by gravitational settling as opposed to centrifugation;
wherein the device is configured such that, upon application of a force, e.g., centrifugal, gravitational, fluid magnetic, electric or fluid, force, detection reagent that has not formed an immune complex can: form a detection reagent complex, e.g., to form an aggregate; migrate into a negative readout region; or, both from a detection reagent complex, e.g., form an aggregate and migrate into a negative readout region.

15. A device for evaluating a sample from a subject, for an anti-RBC antigen antibody, comprising:

a channel comprising
red blood cells, or a first rbcm preparation e.g., a mo-rbcm preparation, disposed on a substantially planar substrate, and the angle between said substantially planar substrate and the direction of applied force, e.g., centrifugal, gravitational, fluid magnetic, electric or fluid, force, that causes migration of detection reagent, is other than 90 degrees;

wherein the device is configured such that, upon application of a force, e.g., centrifugal, gravitational, fluid magnetic, electric or fluid, force, detection reagent that has not formed an immune complex can: form a detection reagent complex, or an aggregate; or, both form a detection reagent complex, or an aggregate.

16. A device for evaluating a sample from a subject, for one or a plurality of different anti-RBC antigen antibodies comprising:

a plurality of channels, each channel comprising
a) a capture region for receiving RBC, a rbcm preparation, or a mimic optimized-rbcm preparation, disposed on a substantially planar substrate, and the angle between said substantially planar substrate and the direction of applied force chosen from a centrifugal, gravitational, fluid magnetic, electric or fluid, force, that causes migration of detection reagent, is other than 90 degrees;

wherein the device is configured such that, upon application of a centrifugal or a gravitational force, the detection reagent that has not formed an immune complex forms a detection reagent complex or an aggregate.

### EXAMPLES

The invention now being generally described, it will be more readily understood by reference to the following examples, which are included merely for purposes of illustration of certain aspects and embodiments of the present invention, and are not intended to limit the invention.

### Example 1

### Antibody Screening Assays

### Background:

This example describes the experimental conditions for an antibody screening assay to detect a red blood cell antigen antibody of G isotype, wherein a red blood cell membrane preparation is bound to a functionalized test surface. In this case, the number of indicator moieties is chosen such that they form sub-monolayer coverage on the bottom of the well plate surface. Although described in the context of antibody screening, optimization of relevant parameters (centrifuge angle, indicator concentration, specificity, etc.) should extend the utility of this invention to any system in which a particle is specifically bound to a surface.

### Materials

The following available materials are used in this example:

(i) Antibody screening panel (Z451), IgG sensitized cells (Z441), O cells (Z416), polyclonal anti-IgG (Z356) obtained from Alba Bioscience;

(ii) Blood bank saline (22-026-401) obtained from Fisher;

(iii) Anti-human IgG, including monoclonal murine IgM from clone MS-278, rabbit polyclonal antibody Alba #Z356, monoclonal antibody Immucor 16H8;

(iv) Thermo: SmartPlex 96 well multiplexing platform [SMARTPLEX-C22-05]

(v) Low Ionic Strength Saline (LISS): 25 mM NaCl, 250 mM glycine, 0.05% sodium azide. pH adjusted to 7.1 with isotonic NaOH. Osmolarity is approximately 280 mOsm

### Preparation of Surfaces:

Suitable glass surfaces are positively charged at neutral pH and free of surface contamination. Any number of surface treatments can be used. For example, a native polystyrene surface can be rendered positively charged via a molecule with a hydrophobic character and an appropriate electrostatic character (for instance, poly-L-lysine). Silica can be rendered positively charged via an amine terminated silane (such as aminopropyltriethoxysilane—APTES). All surfaces should be prepared and stored carefully to avoid fouling of common atmospheric contaminants (hydrocarbons, for instance).

In particular, #1 glass coverglass should be cleaned via chromic sulfuric acid, piranha, or appropriate surfactant to obtain a pristine substrate. The substrate should be rinsed with ultra pure water and dried with compressed air. The glass should be cleaned to a level such that the contact angle between the surface and a distilled/deionized water droplet is less than 5 degrees. The surface can then be amine functionalized using aminopropyltriethoxysilane (APTES) or similar species. The surface can be coated through a CVD process or a liquid dip process. If APTES is utilized, the contact angle between distilled/deionized pH 7.0 water and the surface should be at least 50 degrees. The uniformity of the film can be probed by exposing the surface to amine reactive fluorescent tags such as fluorescein isothiocyanate and examining with fluorescence microscopy.

### Preparation of Screening Cells:

A 2-3% concentration of human red blood cells can be obtained from a commercial supplier, e.g., Alba Bioscience. The human red blood cells can be brought to room temperature using a non-inverting rocker. An aliquot of 60 uL of the red cells can be placed into a 1 mL microcentrifuge tube, and 500 uL saline are added. The microcentrifuge tube is centrifuged at 400 g's for 4 minutes. The supernatant is removed and the cell pellet is re-dispersed in 500 uL saline. These steps can be repeated (about five times) or until the OD at 280 nm<0.01. The supernatant can be removed, and 60 uL saline can be added. 100 uL of saline can be added to a fresh microcentrifuge tube. 10 uL of the washed red cells solution can be added to the fresh microcentrifuge tube.

### Preparation of Plate:

After removing the protective strip on the bottom of a SmartPlex 96 well multiplexing platform [SMARTPLEX-C22-05], a piece of amine-functionalized glass is placed on the bottom of the SmartPlex plate. The plate is heated gently with a heat gun to soften the adhesive material. The amine-functionalized glass is firmly pressed into the softened adhesive with a 100 uL pipette tip. The plate is allowed to cool to room temperature.

### Preparation of Test Surface:

100 uL of the prepared red cell solution is loaded to one of the wells on the SmartPlex plate. The loaded plate is immediately placed into a swinging bucket centrifuged and spun at 400 g's for 5 minutes (20° C.). The surface of the plate is visually inspected to ensure uniform coverage. The test surface is gently washed with saline (3×100 uL) to remove excess cells. 100 uL of distilled/deionized water is added to the well and gently shaken for 1 minute. After lysis is complete, the surface is washed with 100 uL saline.

### Test Procedure (Optical Trapping):

The binding of indicator cells to the prepared surface can be probed using an optical trap. An optical trapping system can be constructed using a collimated 0.5 W 1064 nm continuous wave (CW) laser beam (via a laser such as IPG Photonics # YLR-20-1064-LP) with a beam diameter of 7-12 mm (at objective back aperture) is directed through a Nikon Plan APO 40X (NA 0.95) Air objective mounted in a research grade inverted microscope (Nikon Eclipse TE-200, TE-2000, or Ti or Olympus IX series). The beam diameter and collimation can be controlled using two lenses [Thorlabs LB 1309 and LB 1630] using routine optical alignment techniques familiar to those skilled in the art. The sample is maneuvered via a precision microscope stage.

The test sample is processed as follows. An aliquot of 50 uL of plasma of interest is transferred into a microcentrifuge tube. 50 uL of LISS is added and mixed with the pipettor. 100 uL of the LISS/plasma solution is added to the treated well. The well is covered with Scotch magic tape, and immediately placed into a 37° C. incubator on top of an aluminum plate that is stored permanently in the incubator (heat block) such that good thermal contact and transfer is achieved between the plate and the lower surface of the glass. The LISS/plasma solution is allowed to incubate in the well for the desired time (e.g., 5, 8, 15 minutes). The plate is removed from incubator, and the tape is removed from the well top. The plasma/LISS mixture is extracted from the well using a vacuum aspirator. 100 uL of saline is added to the well drop-wise. The saline is vacuum extracted from the well. These two steps are repeated thrice. 10 uL of a 5% solution of Alba IgG-coated cells is added to 1 mL saline.

An anti-IgG solution is prepared following manufacturer's protocols. 100 uL of the diluted IgG-coated cell solution is added to 1 mL of anti-IgG solution. 100 uL of this indicator cell solution is used to test for the presence of IgG bound to the well surface. The indicator cells are allowed to settle to glass surface by incubating for 3 minutes. Probe binding was detected via optical trapping using the optical trapping system described above.

FIGS. 7A-7B summarize one embodiment of the antibody screening assay. Experimental protocols are as follows. A red blood cell membrane preparation is bound to a functionalized test surface. A sample (e.g., a plasma sample) containing a red blood cell antigen antibody of a G isotype is incubated with the bound red blood cell membrane preparation under conditions that allow for binding, thereby forming a complex. Unbound IgG is reduced by one or more washing steps. An IgG binding reagent (e.g., monoclonal IgM class anti-human IgG from clone MS-278) is added to bind to the complex. A detection agent, e.g., an indicator Alba Bioscience IgG sensitized cells, is added, thereby allowing measurement of the presence of IgG class antibodies which are specific to rbcm antigens from the plasma by way of detection of bound red blood cells, e.g., by detecting binding of the indicator cells to the test surface.

FIG. 7C depicts representative graphs showing the percentage of red blood cells detected as bound using the antibody screening assays described herein as a function of secondary incubation time. FIGS. 7B and 7C depict antigen negative or antibody negative sample/cell. In FIG. 7B, Curve #1 (referred to in the figure as “#1 Cell,” lower/squares) represents the results of an assay using a control anti-RBC antigen antibody-negative sample; curve #2 (referred to in the figure as “#2 Cell,”) represents the results of an assay using an anti-RBC antigen antibody positive sample. In FIG. 7C, Curve #3 represents the results of an assay using a control antigen-negative surface cell; curve #2 represents the results of an assay using an antigen positive surface cell.

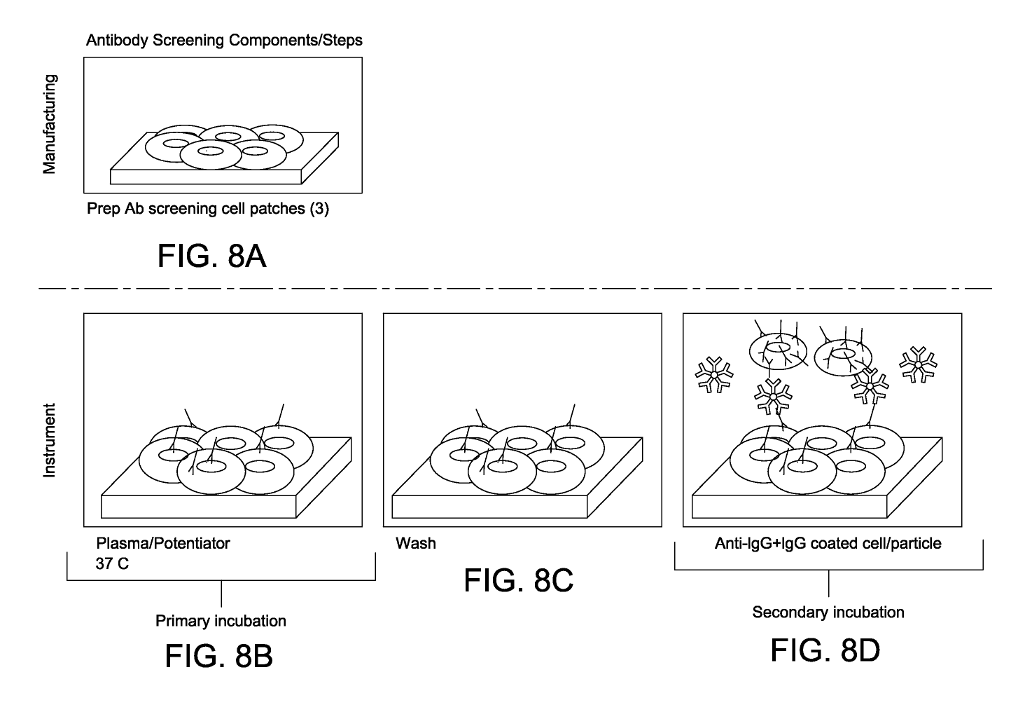

FIGS. 8A-8D provide a stepwise representation of the components of the antibody screening assays described in FIG. 7A. A red blood cell membrane preparation bound to a functionalized test surface is shown FIG. 8A. In FIG. 8B (“primary incubation step”), the rbcm bound test surface is incubated with a plasma sample containing a red blood cell antigen antibody of a G isotype. In FIG. 8C (“wash”), unbound IgG is reduced by one or more washing steps. In FIG. 8D (“secondary incubation step”), a secondary incubation is carried out by adding an IgG binding reagent to bind to the complex and a detection agent, e.g., an indicator Alba Bioscience IgG sensitized cells, thereby allowing measurement of the presence of IgG class antibodies which are specific to rbcm antigens from the plasma by way of detection of binding of the indicator cells to the test surface.

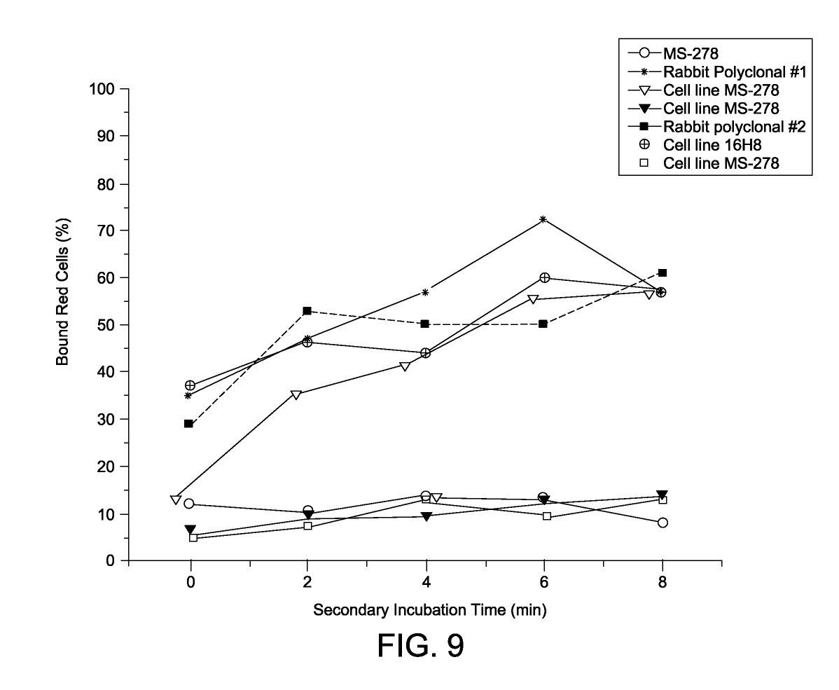

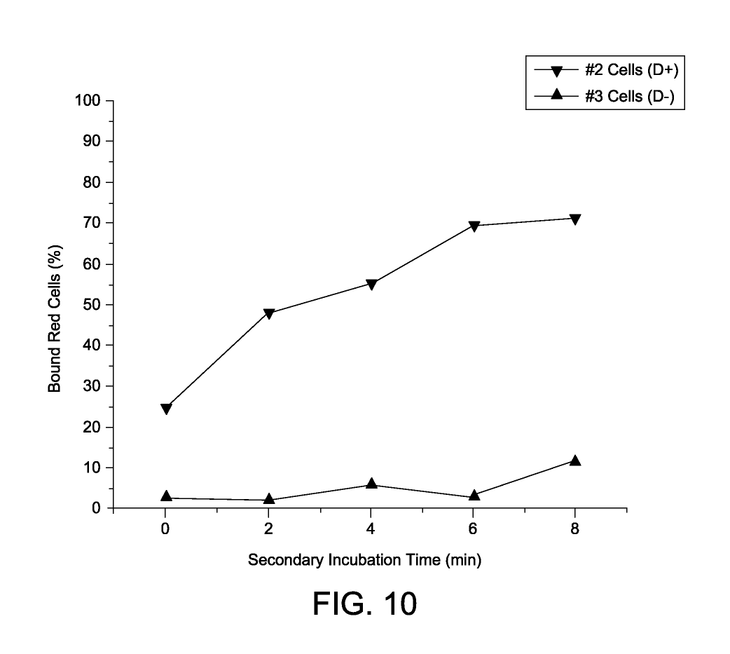

Representative results of antibody screening assays as described herein are shown in FIGS. 9-10. FIG. 9 shows a comparison of the nonspecific binding to a red blood cell membrane preparation using a panel of anti-IgG antibodies. The percentage bound red blood cells detected as a function of secondary incubation time was measured. Each of the anti-IgG antibodies were used at approximately 0.01 mg/mL. In this case, each of MS-278 (also referred to as cell line MS-278), anti-IgG rabbit poly #1 (a Millipore antibody product), anti-IgG rabbit poly #2 (Alba #Z356), and monoclonal antibody Immucor 16H8, was used in its raw bottled (for use in a manual tube test) format. The polyclonal antibodies are a blend of IgG and IgM class antibody (approximately 60-80% IgG). The monoclonal antibodies (Immucor and Millipore) are both IgM class anti-IgG's (both mouse) and are also approximately 0.01 mg/mL. The experimental conditions for this study were as follows. Surface and rbcm preparation were carried out as described above. A candidate anti-IgG antibody (at its optimal concentration) was incubated over the red blood cell coated-surface at various temperatures and times. Typical, temperature and time of incubation were 20° C. and 10 minutes. The red cell surface was washed with normal saline solution (4×200 uL) to remove unbound anti-IgG antibody. Next, IgG-sensitized red cells (Alba Bioscience, at base concentration described above) were added to the test well, and allowed sedimentation of the cells to the test surface for three minutes. Binding was probed with optical trapping.

A significant decrease in non-specific binding to the red cell membrane preparation was detected using the MS-278 anti-IgG antibody compared to the other antibodies tested. These results were reproduced in different experimental runs.

FIG. 10 shows a graph depicting binding of MS-278 monoclonal anti-IgG to two different red blood cell membrane preparations, one positive for the D RBC antigen (#2 Cells D+) and one negative for the D RBC antigen (#3 Cells D−), in the presence of anti-D, as revealed by indicator cells (IgG-coated red cells). The percentage bound red blood cells detected as a function of secondary incubation time was measured. The experimental conditions were carried out at described above. Briefly, once the benchmarks representing limits of detection were established, the titrated plasma sample was incubated over the red blood cell coated surface in conditions known to those skilled in the art. In this case, Alba anti-D proficiency (0.025 IU-#Z264) was used. In particular, the conditions used were 37° C., 15 minutes, 1:1 ratio 0.025 M NaCl (Low Ionic Strength Saline). The test was carried out in parallel such that red blood cell surfaces expressing and not expressing the antigen corresponding to the antibody specificity were examined. The test surfaces were washed with normal saline until sufficiently free of unbound IgG (4×200 uL). Candidate anti-IgG was blended with the IgG-coated red cells and then dispersed over the test surfaces. In particular, Millipore MS-278 was diluted 10 fold with normal saline. IgG coated red cells (Alba #Z441) were diluted 100 fold with normal saline. 100 uL of the IgG-coated cell solution was added to 1 mL of the diluted anti-IgG and then 100 uL of this solution was added to the well. Three minutes were allotted for sedimentation of the cells to the test surfaces. Binding was probed with optical trapping.

### Preparation and Testing of Mimic Optimized RBCM Preparations

FIG. 11 shows a representative graph depicting binding of anti-IgG rabbit poly #2 (Alba #Z356) to non-treated and enzyme treated red blood cell membrane preparations. (1) Surface and red cell preparations were carried out as described above. (2) To enzyme treat surfaces: (a) FabRICATOR® (Genovis #A0-FR1-020) was dissolved in 30 uL DD H2O. (b) 5 uL of this material was diluted into 100 uL 50 mM sodium phosphate 150 mM NaCl pH 6.6, (6) this solution was added to the red blood cell coated surface (post-lysis) and incubated for 30 mins at 37 C, and (7) the surface was washed 4-6 times with 200 uL saline. (3) The rest of the test is as described above.

A significant decrease in non-specific binding to the red cell membrane preparation was detected after treatment of the cell with the FabRICATOR®, compared to non-treated red cell membrane preparations. These results were reproduced in different experimental runs.

### Test Procedure (Centrifuge):

The test sample is processed as follows. An aliquot of 50 uL of plasma of interest is transferred into a microcentrifuge tube. 50 uL of LISS is added and mixed via pipettor. 100 uL LISS/plasma is added to coated the well. The well is covered with Scotch magic tape, and immediately placed into 37° C. incubator on top of an aluminum plate that is stored permanently in the incubator (heat block). The LISS/plasma is incubate for desired time (5, 8, 15 minutes). The plate is removed from incubator, and the tape is removed from the well top. The plasma/LISS mixture is extracted from the well via a vacuum aspirator. 100 uL of saline were added to the well drop-wise. Saline was vacuum extracted from the well. These steps can be repeated twice. 10 uL of Alba IgG-coated cell is added to 1 mL saline. Alba IgG sensitized cells are diluted with saline (1:10 dilution with saline). The anti-IgG antibody is diluted with LISS. Millipore MS-278 monoclonal antibody, rabbit polyclonal antibody Alba #Z356, monoclonal antibody Immucor 16H8 are each used at approximately 1 mg/mL. 800 uL Millipore/LISS material are combined with 100 uL diluted cell material. 100 uL of this solution is added to the well of interest and placed into a swinging bucket centrifuge with a 25 degree inclined plane in the bottom. A strip of lead was placed in the opposite corner of the swinging bucket to counter balance the incline. Spin at 200 g's for 1.5 minutes and 500 g's at 1.5 mins. Read test results via visual inspection/microscopic examination.

FIGS. 16A-16B show two images representative of the centrifuge-based assay described above when a 5 minute primary incubation is used in conjunction with a sample containing anti-D at its limit of detection. FIG. 16A displays the result of this assay when D+ cells are used as the red cell membrane preparation. The surface appears largely uniform and large aggregates are abscent from the center of the well. This result indicates that binding between the indicator cells and red cell preparation on the well is present. This binding prevents large scale agglutination of the indicators. FIG. 16B displays the result of this assay when D− cells are used as the red cell membrane preparation. The surface appears non-uniform and many large aggregates are present in various places on the lower surface of the well. This result indicates that binding between the indicator cells and the red cell preparation on the well is absent. The lack of this binding enables the indicator cells to agglutinate and move rapidly down the well surface.

### Example 2

### Antibody Screening

### Background:

This example describes experimental conditions for an antibody screening assay to detect a red blood cell antigen antibody of G isotype, wherein a red blood cell membrane preparation is bound to a functionalized test surface. In this example, the number of indicator cells added to each well is significantly larger (>3×) than the number of cell required to form a monolayer. The functionalized test surface may consist of a polystyrene surface, modified to promote the adhesion of red blood cells. This may be employed using any number of surface treatments understood to those experienced in the art. Examples include but are not limited to treatment with lectins (i.e. concanavalin A or wheat germ agglutinin from triticum vulgaris), hydrophobic molecules which under certain experimental conditions possess a net positive charge (i.e. Alcian Blue/yellow or poly-1-lysine) or more elaborate measures including a plasma treatment followed by chemical grafting of silanes. All surfaces should be prepared and stored carefully to avoid surface fouling caused by common atmospheric contaminants (e.g. hydrocarbons). Although described in the context of antibody screening, optimization of relevant parameters (centrifuge angle, indicator concentration, specificity, etc.) should extend the utility of this invention to any system in which a particle is specifically bound to a surface.

### Materials:

The following materials are used in this example:
i. Antibody screening panel (Z451), IgG sensitized cells (Z441), O cells (Z416), polyclonal anti-IgG (Z356) obtained from Alba Bioscience;
ii. Blood bank saline (22-026-401) obtained from Fisher;
iii. Anti-human IgG, including monoclonal murine IgM from clone MS-278, rabbit polyclonal antibody Alba #Z356, monoclonal antibody Immucor 16H8;
iv. Microlon 200 96 well medium bind plate (762070) and microplate lid (656170) from Grenier Bio-one;
v. Low Ionic Strength Saline (LISS): 25 mM NaCl, 250 mM glycine, 0.05% sodium azide with a pH adjusted to 7.1 with isotonic NaOH. Osmolarity of approximately 280 mOsm
vi. Zero Ionic Strength Solution (ZISS): 300 mM glycine, 0.05% sodium azide with a pH adjusted to 7.1 with isotonic NaOH. Osmolarity of approximately 280-300 mOsm;
vii. Ultra Low Ionic Strength Saline (ULISS): 1 part LISS+49 parts ZISS;
viii. BupH phosphate buffered saline packs (28372) from Thermo Scientific;
ix. Alcian blue 8GX (A5268), Methanol (322415), and Minipax absorbent packets (Z163589) from Sigma-Aldrich;
x. VWR vacuum filtration system with 0.2 um PES membrane (87006-062);
xi. Modified Alservere's solution 100 g dextrose, 40 g trisodium citric acid, 10 g NaCl, 4.69 g inosine, 0.1 g citric acid, 1.7 g chloramphenicol, 0.5 g neomycin sulfate—volume to 5 L with distilled water.

### Preparation of Alcian Blue 8GX Solution:

A 1 mg/mL solution of Alcian Blue 8GX is prepared by mixing equal parts methanol and saline. For example, if one mixes 200 mL of methanol with 200 mL of saline then 400 mg of Alcian Blue 8GX should be added to the methanol/saline solution and mixed well (i.e. mixed until when the container is inverted there is no solid on the bottom of the vessel nor visible solid suspended in solution). The solution is filtered using a 0.2 um PES membrane vacuum filtration system from VWR.

### Preparation of Surface:

The prepared Alcian solution is delivered to the polystyrene well plate (100 uL per well) and the plate is covered with a plate lid. The Alcian solution is incubated with the well plate at 4° C. for 12-24 hours. Following incubation, the plates are allowed to warm to room temperature and then unbound Alcian is removed through washing. The wash steps include tapping the Alcian solution out of the plate followed by sequential washing by immersion and shaking in bins containing deionized water (twice) and saline. Following the saline wash the plates are vacuum aspirated and stored in heat sealed mylar bags under nitrogen with desiccant packs. Each mylar bag contains 1-3 plates with approximately 10 grams of desiccant.

### Preparation of Screening Cells:

A 2-5% concentration of human red blood cells can be obtained from a commercial supplier, e.g. Alba Biosciences. The human red blood cells can be brought to room temperature and re-suspended in the bulk solution using a non-inverting rocker. The cells may be washed with saline until the supernatant of the cell solution following centrifugation has an OD at 280 nm<0.01 absorbance units. The cells should be diluted with saline to a final concentration of approximately 0.3%.

### Preparation of Test Surface:

The test surfaces are prepared using a Biotek EL406 and a Beckman Coulter swinging bucket centrifuge. All wash/solution handling/agitation steps mentioned in this section are performed using the Biotek EL406. The surface is prepared by delivering 100 uL of an approximately 0.3% solution of prepared red cell solution to each of the desired wells of a Alcian modified well plate. The loaded plate is immediately placed into a swinging bucket centrifuge and spun at 500 g's for 5 minutes (20° C.). Following centrifugation, the unbound/loosely bound red cells are removed through a series of plate agitations and saline washes. This series entails first agitating the plate for 40 seconds followed by four cycles consisting of a 200 uL saline wash followed by 10 seconds of plate agitation. Following the last cycle the plate is washed with an additional 200 uL of saline per well. Cell lysis is then performed using distilled/deionized water under the following conditions: two wash cycles with 200 uL and one with 50 uL distilled/deionized water per well, 30 seconds of plate agitation, followed by a wash with 200 uL distilled/deionized water. Following the final wash, the water solution is replaced with Modified Alsevere's storage solution, covered with a plate lid and stored at 4° C.

### Test Procedure (Centrifuge):

The test sample is processed as follows. A 50 uL aliquot of plasma to be tested is transferred to a microcentrifuge tube. To this, 50 uL of ULISS is added and mixed via pipettor. 100 uL of the ULISS/Plasma is added to the test well. The well is covered with Scotch magic tape or a plate lid, and immediately placed into a 37° C. incubator on top of an aluminum plate that is stored permanently in the incubator (heat block). The well plate with plasma/ULISS is incubated for a desired time (5, 8, 15 minutes). The plate is removed from the incubator, the lid/tape is removed, and test well is washed. The washing involves the extraction of the plasma/ULISS mixture via a vacuum aspirator followed by repeated washings with 100 uL of saline (added drop-wise) followed by vacuum aspiration. These steps may be repeated twice.

A solution of Anti-IgG, preferably monoclonal murine IgM from clone MS-278, is prepared in BupH PBS or saline at a ratio of between 1:20-1:50. This solution is sufficiently mixed to ensure a homogeneous mixture is achieved. Alba IgG sensitized cells are rocked or mixed to ensure re-suspension of cells followed by addition of 5-10% volume of sensitized cells to the volume of IgM solution. This solution is mixed and 200 uL is added to the test well. The plate is placed into a swinging bucket centrifuge with a 10 degree inclined plane in the bottom. A strip of lead was placed in the opposite corner of the swinging bucket to counter balance the incline. Spin at 80 g's for one minute and 500 g's for 3 minutes. Read the test results via visual inspection/microscopic examination.

FIGS. 16C-16D show two images representative of the centrifuge-based assay described above when a 5 minute primary incubation is used in conjunction with a sample containing anti-D at its limit of detection. FIG. 16C displays the result of this assay when D+ cells are used as the red cell membrane preparation. The surface appears uniform and large tears or defects are absent from the well. This result indicates that a bridge between the indicator membrane and the red cell preparation on the well surface is present. This is the test result obtainted from a weakly positive antibody screen test. FIG. 16D displays the result of this assay when D− cells are used as the red cell membrane preparation. The surface appears non-uniform and large tears and open areas are present across much of the well. This result indicates that a bridge between the indicator membrane and the red cell preparation on the well surface is absent. This is the test result obtained from a negative antibody screen test.

### Example 3

### ABO Reverse Grouping Assays

### Background:

This example describes experimental conditions for a solid-phase ABO reverse grouping assay. Although described in the context of ABO reverse grouping, similar conditions can be applied to any immunoassay where an erythrocyte antigen specific IgM immunoglobulin needs to be detected.

In a conventional reverse typing assay, A₁ and B cells are typically combined with patient plasma in a U or round bottom microtiter plate. The plate is centrifuged and then agitated to disperse non-agglutinated cells.

Another pathway is to immobilize A1 and B cells to the bottom of a flat bottom, round bottom, or U bottom microtiter plate. This pathway can simplify the hardware requirements required to automate these assays. To perform the test, plasma is added to wells pre-coated with lysed A1 and B cells, and then intact A1 and B cells are added to the appropriate wells. The plate is centrifuged and read. If binding occurs (indicating the presence of anti-A or anti-B), the indicator cells will remain dispersed across the bottom of the well plate surface. If binding is absent, the indicator cells will pellet (in the case of round or U bottom well plate) or travel to the bottom-most portion of the flat bottom plate (if centrifuged at a non-zero angle)

Experiments performed by Applicants show that indicator cells can mistakenly appear to bind to the surface, even in the absence of specific antibodies. This particular effect is mostly evident when forward typing and reverse grouping are both conducted on the same disposable. To remove these non-bound cells, surface cells of RhD negative phenotype and indicator cells of RhD positive phenotype (anti-D IgM is added to the test plasma in addition to the RhD positive indicator cells) were used. Use of anti-D IgM causes indicator cells which are not bound to a surface to agglutinate and subsequently travel to the lowest edge of the well during centrifugation, thus eliminating apparent false positives. Importantly, the surface cells are of RhD negative phenotype, and thus no anti-D induced surface/indicator interaction was observed.

### Materials:

The following available materials are used in this example:
(i) A1 Rh(D−), B Rh(D)−, O Rh(D)− cells (Z401, Z411, Z421) obtained from Alba Bioscience
(ii) A Rh(D) Positive, B Rh(D) Positive, O Rh(D) Positive cells obtained from Heartland Blood Center, Aurora Ill.
(ii) Blood bank saline (22-026-401) obtained from Fisher;
(iii) Anti-D (Z031) obtained from Alba Bioscience
(iv) Round bottom microtiter plate (767-070) obtained from Greiner BioOne
(v) Modified Alsevere's solution: 100 g dextrose, 40 g trisodium citric acid, 10 g NaCl, 4.69 g inosine, 0.1 g citric acid, 1.7 g chloramphenicol, 0.5 g neomycin sulfate—volume to 5 L with distilled water.
(vi) Poly-L-Lysine HBr (P1524) obtained from Sigma-Aldrich

### Preparation of Plate:

For deposition of erythrocytes, suitable surfaces include several organic polymers and glass which is modified to carry a positive charge to keep the erythrocytes adsorbed through the deposition, washing, drying and testing process. Specifically, to each well of a Greiner medium binding 96 well “strip” polystyrene plate (Microlon-200), 100 uL of 0.5 mg/mL poly-1-lysine is added, sealed with tape and stored overnight at 4° C. When ready to use, each well within a strip is aspirated and washed three times with 200 uL of 0.9% saline.

### Preparation of Test Surface:

To the poly-1-lysine treated wells, 100 uL of a 1% solution of Rh(D) negative, washed A1, B, or O cells in 0.9% saline are added to separate wells. The cells are then centrifuged at 400 g for 5 minutes and washed three times with 200 uL 0.9% saline. The cells are next lysed by the addition of 200 uL of water for two minutes, followed by two washes with 200 uL water. After the last wash 75 uL of a solution composed of 0.2% anti-D (Alba Z031) in BupH PBS is added to the well. The plate is then ready for use.

### Test Procedure:

To carry out the test, a sufficient amount of plasma (typically 50-75 uL) is added to the test well, followed by 4 uL of a 3% solution of A, B, or O cells. The plate was placed into a swinging-bucket centrifuge and spun at 200 g for 1.5 minutes and 500 g for an additional 1.5 minutes. The plate is then examined for both adherent cells (monolayer to multilayer with uniform coating) and non-adherent cells (in the case of a round or U bottom plate, a pellet).). If the plasma contains antibodies to the A antigen, there will be a uniform layer of cells stuck within the “A” well. Likewise, if the plasma contains antibodies to the B antigen, there will be a uniform layer of cells stuck within the “B” well. In the event that you have cells stuck in the “O” well, this indicates the presence of antibody to the H antigen, and thus it is likely the patient would be of the Bombay phenotype.

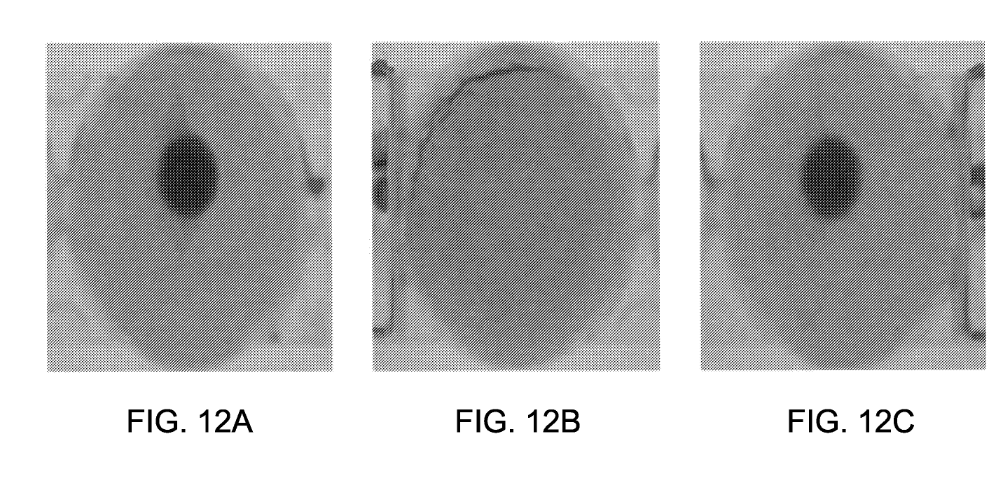

FIGS. 12A-12C shows a representative panel of photographs depicting the readout of the ABO reverse grouping assays described above. FIG. 12A contains A+ indicator cells and a pellet is obvious indicating that the sample does not contain anti-A. FIG. 12B contains B+ indicator cells and a pellet is absent indicating that the sample contains anti-B. FIG. 12C contains O+ indicator cells and a pellet is present indicating that the sample does not contain anti-H or other confounding antibodies.

### Example 4

### Forward Typing

### Materials:

The following available materials are used in this example:
(i) Anti-A, anti-B, anti-D—material from cell lines LA2, LB2, LDM1 obtained from Alba Biosciences—purified to greater than 90% and concentrated to 1 mg/mL
(ii) Round bottom microtiter plate (767-070) obtained from Greiner BioOne
(iii) Blood bank saline (C) obtained from Fisher Scientific
(iv) BSA (A7906) obtained from Sigma-Aldrich
(v) BupH PBS (28372) obtained from Pierce
(vi) Poly-1-lysine (P1524) obtained from Sigma-Aldrich
(vii) Tween 20 (P9416) obtained from Sigma-Aldrich
(viii) 96 well microtiter plate lid (656-170) obtained from Greiner BioOne

### Preparation of Antibody Solutions:

20 mL of BupH PBS is added to each of three tubes and the tubes are marked as “LA2”, “LB2”, and “LDM1”. Each tube receives 80 μL of purified anti-A (LA2), anti-B (LB2) or anti-D (LDM1) (each antibody into respectively marked tube) and the tubes are thoroughly mixed.

### Preparation of Test Surface:

Greiner medium binding round bottom 96 well plates are loaded with 100 uL of the appropriate antibody solution. The plate is covered with a lid and stored in a refrigerator at 4 C overnight. The next morning, the plate is then washed at least six times with 200 uL of saline to remove unbound protein.

### Plate Blocking:

The wells are then aspirated and 200 uL of a blocking solution (3% BSA, 0.1% Tween 20 in BupH PBS) is added to each. This is repeated for all rows. The plate is then covered with a lid and stored at 4 C for 36 hours. After this time has elapsed, the plate is ready for use.

### Preparation of Red Cells:

Red blood cells are diluted into 0.9% saline to a final concentration of 0.04% (i.e. first 10 uL packed RBCs mixed with 90 uL 0.9% saline and 4 uL of this dilution is mixed with 1000 uL 0.9% saline).

### Test Procedure:

100 uL of the 0.04% RBC solution is added to one well of LA2,1 LB2 and LDM1. The strip (being held by the 96-well plate frame) is placed into a swinging bucket centrifuge and spun for 1.5 minutes at 200 g's and an additional 1.5 minutes at 500 g's.

### Result Interpretation:

The plate is then examined for binding—a negative binding event is designated as the formation of a red cell button in the well; a positive binding event is designated as the lack of a red cell button (there is a red “haze” present from the red cells binding over the surface of the well).

FIGS. 1E-1G display a typical result of this assay. FIG. 1E is an image of a well coated with anti-A as described herein, exposed to sample, and then centrifuged. FIG. 1E shows a ‘haze’ of blood cells indicating that binding between the cells and the surface is present and that the cells in the sample present the A antigen. FIG. 1F is an image of well coated with anti-B as described herin, exposed to sample, and then centrifuged. The figure shows a pellet of red blood cells indicating that binding between the cells and the surface is absent. Thus, the cells in the sample do not present the B antigen. FIG. 1G is an image of the well coated with anti-D as described herein, exposed to sample, and then centrifuged. The figure shows a ‘haze’ of blood cells indicating that binding between the cell and the surface is present. Thus, the cells contained in the sample present the D antigen. Therefore, the blood type of this particular sample may be interpreted as A+.

### Example 5

### Minor Antigen Typing and Extended Phenotyping

Materials:

(i) Protein L from Peptostreptococcus magnus (P3101) obtained from Sigma Aldrich

(ii) Anti-D (Z031) obtained from Alba Bioscience

(iii) Anti-c (Z083) obtained from Alba Bioscience

(iv) Anti-C (Z063) obtained from Alba Bioscience

(v) Anti-e (Z094A) obtained from Alba Bioscience

(vi) Anti-E (Z073) obtained from Alba Bioscience

(vii) Anti-Jka (Z162) obtained from Alba Biosceince

(viii) BupH phosphate buffered saline obtained from Pierce

(ix) Blood bank saline (22-026-401) obtained from Fisher Scientific

(x) Round bottom 96 well plates (767-070) obtained from Greiner BioOne

(xi) 96 well plate lids (656-170) obtained from Greiner BioOne

### Plate Preparation:

Protein L was dissolved in PBS at a concentration of 1 mg/mL. It was then diluted 5000-fold with PBS and 75 uL of this solution was pipetted into each well. The plate was covered with a lid and allowed to incubate overnight at 4 C. After incubation was complete, each well was washed with 200 uL of saline 5× and then 75 uL of the desired antibody (anti-D, anti-c, anti-C, anti-e, anti-E, anti-Jka) was added to each well and the reaction allowed to proceed for 4 hours at room temperature. The wells were once again washed with 200 uL of saline 4×.

### Test Procedure:

100 uL of a 0.04% suspension of test red blood cells are added to each well. The plate is centrifuged at 50 g's for 8 mins in a swinging bucket centrifuge and then the plate is read. A tightly packed pellet at the bottom of the well indicates that the sample is negative for the antigen in question, and a dispersed or ‘hazy’ layer of test cells indicates a positive.

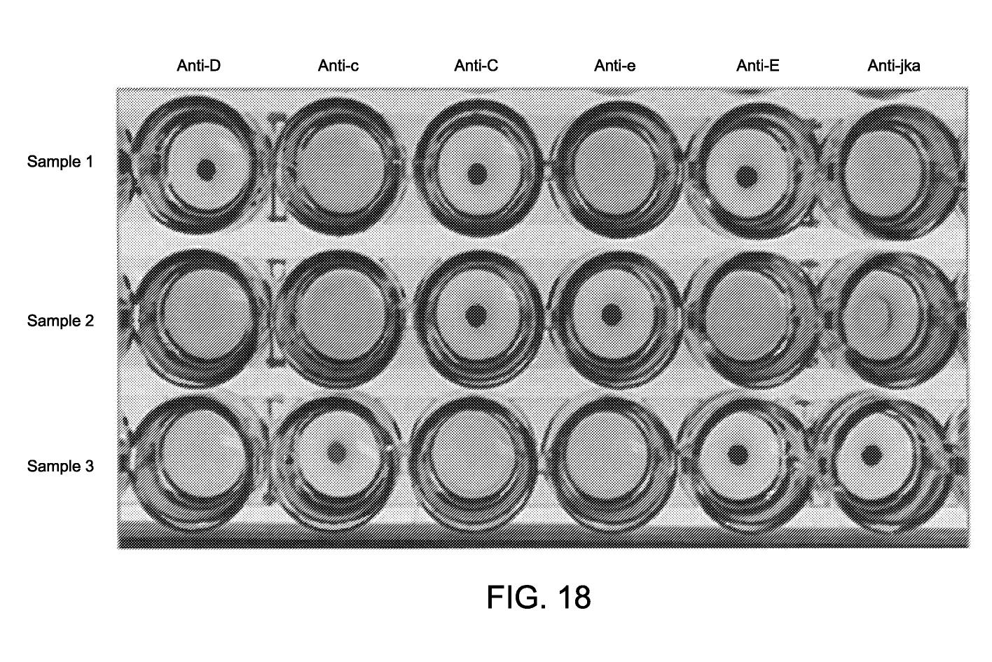

FIG. 18 is an image of three samples tested using the assay described herein. Each row represents one distinct sample. Each column represents one distinct specificity (D, c, C, e, E, Jk^{a}). The figure shows that Sample 1 has the following antigen profile D−, c+, C−, e+, E−, Jka+. Sample 2 has the following antigen profile: D+, c+, C−, e−, E+, Jka+. Sample 3 has the following antigen profile: D+, c−, C+, e+, E−, Jka−.

### Example 6

### Sequencing of Monoclonal Antibody MS-278

This example describes experimental conditions used for sequencing of the variable region of the IgM monoclonal antibody MS-278.

The materials and methods used in this example are as follows:

### Reduction and Alkylation of Disulfide Bonds

Protein samples were re-solubilized in 50 mM triethylammonium bicarbonate (TEAB) buffer prior to reduction by addition of tris(2-carboxyethyl)phosphine (TCEP) to a final concentration of 5 mM and incubation at 37° C. for 20 min. Subsequently io doacetamide to a 10 mM final concentration was added and the sample was incubated at room temperature for another 20 mins in the dark.

### SDS-PAGE

To separate the two species of antibody subunits (LC, HC) the antibody sample was solubilized in sample loading buffer (Lämmli, 1970). Aliquots of 5 μg sample were loaded onto an SDSPAGE gel. After the gel run (150 V, max. 400 mA, 75 min) the gel was incubated in 50% ethanol, 10% acetic acid for 30 min prior to gel staining with Coomassie Brilliant Blue (CBB G250) according to standard techniques.

### In Gel Enzymatic Cleavage

Gel slices from SDS-PAGE gels were prepared to enzymatic cleavage by 3 times swelling/shrinking in 100 mM ABC or 50 mM ABC, 60% ACN respectively. Each step was carried out for 30 min at room temperature. After the last shrinking step the gels slices were dried in open eppendorf cups for 15 min. Proteolysis was started by adding 3 volumes of enzyme solutions with an enzyme/protein ratio of 1:50. Table 2 lists the enzyme solutions used for the proteolyses.

Table 2: List of proteolytic enzymes with their appropiate buffer solutions and incubation temperatures.
Tr/TL/PK/Elastase: 50 mM ammonium bicarbonate, 10% acetonitrile (v/v) @ 37° C.
CT: 100 mM Tris-HCl, 10 mM CaC12, 5% ACN (v/v), pH 8.0 @ 37° C.
LysC: 50 mM Tris-HCl, 1 mM EDTA, 10% ACN (v/v), pH 8.5 @ 37° C.

### GluC: 50 mM Tris-HCl, 0.5 mM Glu-Glu, pH 8.0 @ 25° C.

Each proteolysis was carried out over night. The resulting peptides were acidified with 1/2 volume of 2% FA prior to mass spectrometry.

Proteolysis in Solution with Cysteine Derivatization

The protein samples were denatured by 8M urea with 5 mM TCEP for 30 minutes. Next IAA to a final concentration of 10 mM was added and the reaction was incubated 30 min in the dark at room temperature. After dilution to 0.8M urea with appropriate protease buffer (Table 2), the sample was digested separately by endoproteases (trypsin, chymotrypsin, GluC or LysC, respectively; enzyme to protein ratio (w/w): 1:50) according to standard procedures.

### High-Resolution Mass Spectrometry

The HPLC system was coupled to an Advion NanoMate 100 chip-electrospray system (Advion, Ithaca, N.Y.), and detection was performed on a Finnigan LTQ-FT mass spectrometer (ThermoFisher, Bremen, Germany) equipped with a 6T magnet.

Samples from proteolyses were applied to nanoLC-ESI-MS/MS after acidification. After trapping and desalting the peptides on enrichment column (Zorbax SB C18, 0.3 mm×5 mm, Agilent) using 1% acetonitrile/0.5% formic acid solution for five minutes peptides were separated on Zorbax 300 SB C18, 75 μm×150 mm column (Agilent, Waldbronn) using an acetonitrile/0.1% formic acid gradient from 5% to 40% acetonitrile. MS overview spectra were automatically taken in FT-mode according to manufacturer's instrument settings for nanoLC-ESI-MSMS analyses, peptide fragmentation and detection was accomplished in the instrument's LTQ ion trap.

Beside one dimensional nanoLC-ESI-MSMS several analyses were performed by twodimensional nanoLC-ESI-MSMS (MudPIT) using a strong cation column (SCX) online coupled to the C18 trapping column. By increasing NaCl salt steps (10-300 mM) peptides which were previously trapped to the SCX column were eluted to the C18 trap column before nanoLC-ESIMSMS analysis.

### HPLC Separation and Edman Sequencing

Peptides were separated and fractionated by an Agilent 1100 HPLC system using a Phenomenex Kinetex C18 column with a water/acetonitrile/0.1% TFA gradient according to standard procedures. Peptide fractions were applied to an Applied Biosystems Procise 494 Edman sequencer for amino acid sequencing.

### Database Searches

Data sets acquired by high-resolution mass spectrometry were used for database searches against a custom database of known antibody sequences utilizing the Mascot search engine (Matrix Science Ltd., London) or OMSSA. The databases used were either derived from the constantly updated NCBInr database or generated in-silico. The search parameters were set according to the expected protein modifications and to the MS instrument used in this study.

### In-Silico Database Generation

In-silico databases were produced with respect to their calculated size and complexity. For databases containing a final set of 20̂6 or less sequence candidates a brute force algorithm was used to generate the full set of sequences.

For databases that would contain more than 20̂6 sequences and therefore exceed current computation and storage capacities a two step methodology was used. In a first step, anagram-like isobaric peptide sequences were reduced to a set of degenerated pre-candidates. The data reduction of sequence candidates can be exemplified as follows.

### ANNA-anchor

### NANA-anchor

ANAN-anchor [symbol] 2A2N-anchor

### NAAN-anchor

### NNAA-anchor

### AANN-anchor

complete set [symbol] degenerated single pre-candidate
anchor=known conserved/indentified sequence tag

The set of candidates from the first step were used for a database search. Pre-candidates matching MS data were manually reviewed and selected according to the cleavage enzymes specifity, known consensus sequences in front and behind antibodies' CDR regions and fragment ions of the fixed anchor sequence. Selected pre-candidates were used for the regeneration of a complete sequence set (as exemplified above) and used for a database search. This procedure was iteratively repeated.

### Example 7

### Amino Acid Sequences of the Variable Region of Monoclonal Antibody MS-278

This example describes the amino acid sequence analysis of the IgG light and heavy chain variable regions of monoclonal antibody MS-278.

### Light Chain Variable Region

The amino acid sequence of the light chain variable region shown below was derived from analytical data. The amino acid sequence is a composite of peptides detected after the different proteolytic digests described in Example 6. CDR regions are underlined and indicated in bold. “X” indicates non-detected sequence parts.

(SEQ ID NO: 1)

| | |
|---|---|
| 1 | DIVLTQSPASLAVSLGQRATISCRASESVDSYGNSFMHWY |
| 41 | QQKPGQPPKLLIYRASNLESGIPARFSGSGSGTDFTLTIN |
| 81 | PVEADDVATYYCQQTNEDPRTFGGGTKLELKRADAAPTVS |
| 121 | IFPPSSEQLTSGGASVVCFLNNFYPKDINVKWKIDGSERQ |
| 161 | NGVLNSWTDQDSKDSTYSMSSTLTLTKDEYERHNSYTCEA |
| 201 | THKTSTSPIVKSF |

CDR1 of the light chain variable region includes the following sequence:

(SEQ ID NO: 2)
RASESVDSYGNSFMH

CDR2 of the light chain variable region includes the following sequence:

(SEQ ID NO: 3)
RASNLES

CDR3 of the light chain variable region includes the amino acid sequence:

(SEQ ID NO: 4)
DPRT.

In one embodiment, the CDR3 of the light chain includes the amino acid sequence

(SEQ ID NO: 7)
QQTNEDPRT.

CDR3 can have the following consensus sequence:

(SEQ ID NO: 5)
X₁ X₂ X₃ X₄ X₅ X₆ D P R T,

wherein:
X₁=Q, A, G, or absent;
X₂=A, G, F, Q, or absent;

### X₃=G, Q, P, Q, A or T;

### X₄=T, L or G;

### X₅=N, E or G; and

### X₆=E, N or V.

Exemplary sequences for CDR3 of the light chain variable region include:

(SEQ ID NO: 6)
QAGTNEDPRT
(SEQ ID NO: 7)
QQTNEDPRT
(SEQ ID NO: 8)
AGQTENDPRT
(SEQ ID NO: 9)
AGQTNEDPRT
(SEQ ID NO: 10)
FPLGVSDPRT
(SEQ ID NO: 11)
GAQTENDPRT
(SEQ ID NO: 12)
QGATNEDPRT
(SEQ ID NO: 13)
QQTGGEDPRT

### Heavy Chain Variable Region

The amino acid sequence of the heavy chain variable region shown below was derived from analytical data. The amino acid sequence is a composite of peptides detected after the different proteolytic digests described in Example 6. CDR regions are underlined and in bold. “X” indicates non-detected sequence parts.

| (SEQ ID NO: 14) | |
|---|---|
| 1 | QVTLKESGPG ILQPSQTLSL TCSFSGFSLS TSGMGVSWIR QPSGKGLEWL |
| 51 | AHIYWDDDKR YNPSLKSRLT ISKDTSRNQV FLKITSVDTA DTATYYCARx |
| 101 | xxxxxxLDYW GQGTTLTVSS ESQSFPNVFP LVSCESPLSD KNLVAMGCLA |
| 151 | RDFLPSTISF TWNYQNNTEV IQGIRTFPTL RTGGKYLATS QVLLSPKSIL |
| 201 | EGSDEYLVCK IHYGGKNRDL HVPIPAVAEM NPNVNVFVPP RDG |

CDR1 of the heavy chain variable region can have the following consensus sequence:

(SEQ ID NO: 15)
X₁ X₂ X₃ S L S T S G M G V S,

wherein

### X₁ is G or Y;

### X₂ is F, G or Y; and

X₃ is A or absent.
CDR1 of the heavy chain can have one of the following amino acid sequences:

(SEQ ID NO: 16)
GFSLSTSGMGVS
(SEQ ID NO: 17)
GYASLSTSGMGVS
(SEQ ID NO: 18)
YGASLSTSGMGVS
(SEQ ID NO: 19)
MEEFLL
(SEQ ID NO: 20)
LLLFGL
or
(SEQ ID NO: 21)
NSDYYK.

CDR2 of the heavy chain variable region includes the following sequence:

(SEQ ID NO: 22)
HIYWDDDKRYNPSLKS.

Exemplary candidate sequences for CDR2 of the heavy chain variable region include one of the following amino acid sequences:

(SEQ ID NO: 23)
DYVQEDISKDTSR
(SEQ ID NO: 24)
FMVQEDISKDTSR
(SEQ ID NO: 25)
DYNLEDISKDTSR
(SEQ ID NO: 26)
LFFVPHISK
(SEQ ID NO: 27)
RHNVPHISK
(SEQ ID NO: 28)
AALQELISK
(SEQ ID NO: 29)
FKTVDRTISKD
(SEQ ID NO: 30)
GYRVDRTISKD
(SEQ ID NO: 31)
VEAFQTTISK
(SEQ ID NO: 32)
NNAFKTTISK
(SEQ ID NO: 33)
DIAFQTTISK,
or
(SEQ ID NO: 34)
YPEAWETISK.

CDR sequences are bolded and underlined.
CDR3 of the heavy chain variable region can include one of the following amino acid sequences:

(SEQ ID NO: 35)
TYYCARTTGY
(SEQ ID NO: 36)
TYYCARSDGY,
or
(SEQ ID NO: 37)
DYWGQGTSVTVSS.

In certain embodiments, the CDR3 sequence of the heavy chain variable region comprises, consists essentially of, or consists of, the following amino acid sequence: ARSDGYYHYAMLDY (SEQ ID NO:38).

An exemplary heavy chain variable can include the following amino acid sequence.

| | (SEQ ID NO: 39) |
|---|---|
| 1 | QVTLKESGPG ILQPSQTLSL TCSFSGFSLS TSGMGVSWIR QPSGKGLEWL |
| 51 | AHIYWDDDKR YNPSLKSRLT ISKDTSRNQV FLKITSVDTA DTATYYCARS |
| 101 | DGYYHYAMLD YWGQGTSVTV SS ESQSFPNVFP LVSCESPLSD KNLVAMGCLA |
| 151 | RDFLPSTISF TWNYQNNTEV IQGIRTFPTL RTGGKYLATS QVLLSPKSIL |
| 201 | EGSDEYLV CKIHYGGKNR DLHVPIPAVAEM NPNVNVFVPP RDG. |

The approximate location of the CDR sequences are indicated by the underline and in bold.

TABLE 1
Blood group antigens within systems
. . . = obsolete; provisional numbers are in italic

| | | Antigen Number | | | | | | | | | | | |
|---|---|---|---|---|---|---|---|---|---|---|---|---|---|
| System | | 001 | 002 | 003 | 004 | 005 | 006 | 007 | 008 | 009 | 010 | 011 | 012 |
| 001 | ABO | A | B | A, B | A1 | . . . | | | | | | | |
| 002 | MNS | M | N | S | s | U | He | Mi^{a} | M^{c} | Vw | Mur | M^{g} | Vr |
| 003 | P | P1 | . . . | . . . | | | | | | | | | |
| 004 | RH | D | C | E | c | e | f | Ce | C^{w} | C^{x} | V | E^{w} | G |
| 005 | LU | Lu^{a} | Lu^{b} | Lu3 | Lu4 | Lu5 | Lu6 | Lu7 | Lu8 | Lu9 | . . . | Lu11 | Lu12 |
| 006 | KEL | K | k | Kp^{a} | Kp^{b} | Ku | Js^{a} | Js^{b} | . . . | . . . | Ul^{a} | K11 | K12 |
| 007 | LE | Le^{a} | Le^{b} | Le^{ab} | Le^{bH} | ALe^{b} | BLe^{b} | | | | | | |
| 008 | FY | Fy^{a} | Fy^{b} | Fy3 | Fy4 | Fy5 | Fy6 | | | | | | |
| 009 | JK | Jk^{a} | Jk^{b} | Jk3 | | | | | | | | | |
| 010 | DI | Di^{a} | Di^{b} | Wr^{a} | Wr^{b} | Wd^{a} | Rb^{a} | WARR | ELO | Wu | Bp^{a} | Mo^{a} | Hg^{a} |
| 011 | YT | Yt^{a} | Yt^{b} | | | | | | | | | | |
| 012 | XG | Xg^{a} | CD99 | | | | | | | | | | |
| 013 | SC | Sc1 | Sc2 | Sc3 | Rd | STAR | SCER | SCAN | | | | | |
| 014 | DO | Do^{a} | Do^{b} | Gy^{a} | Hy | Jo^{a} | DOYA | | | | | | |
| 015 | CO | Co^{a} | Co^{b} | Co3 | | | | | | | | | |
| 016 | LW | . . . | . . . | . . . | . . . | LW^{a} | LW^{ab} | LW^{b} | | | | | |
| 017 | CH/RG | Ch1 | Ch2 | Ch3 | Ch4 | Ch5 | Ch6 | WH | | | | Rg1 | Rg2 |
| 018 | H | H | | | | | | | | | | | |
| 019 | XK | Kx | | | | | | | | | | | |
| 020 | GE | . . . | Ge2 | Ge3 | Ge4 | Wb | Ls^{a} | An^{a} | Dh^{a} | GEIS | | | |
| 021 | CROM | Cr^{a} | Tc^{a} | Tc^{b} | Tc^{c} | Dr^{a} | Es^{a} | IFC | WES^{a} | WES^{b} | UMC | GUTI | SERF |
| 022 | KN | Kn^{a} | Kn^{b} | McC^{a} | Sl1 | Yk^{a} | McC^{b} | Sl2 | Sl3 | KCAM | | | |
| 023 | IN | In^{a} | In^{b} | INFI | INJA | | | | | | | | |
| 024 | OK | Ok^{a} | | | | | | | | | | | |
| 025 | RAPH | MER2 | | | | | | | | | | | |
| 026 | JMH | JMH | JMHK | JMHL | JMHG | JMHM | | | | | | | |
| 027 | I | I | | | | | | | | | | | |
| 028 | GLOB | P | | | | | | | | | | | |
| 029 | GIL | GIL | | | | | | | | | | | |
| 030 | RHAG | Duclos | Ol^{a} | Duclos- | | | | | | | | | |
| | | | | like | | | | | | | | | |
| | | Antigen Number | | | | | | | | | | | |
| System | | 013 | 014 | 015 | 016 | 017 | 018 | 019 | 020 | 021 | 022 | 023 | 024 |
| 002 | MNS | M^{e} | Mt^{a} | St^{a} | Ri^{a} | Cl^{a} | Ny^{a} | Hut | Hil | M^{v} | Far | s^{D} | Mit |
| 004 | RH | . . . | . . . | . . . | . . . | Hrₒ | Hr | hr^{S} | VS | C^{G} | CE | D^{w} | . . . |
| 005 | LU | Lu13 | Lu14 | . . . | Lu16 | Lu17 | Au^{a} | Au^{b} | Lu20 | Lu21 | | | |
| 006 | KEL | K13 | K14 | . . . | K16 | K17 | K18 | K19 | Km | Kp^{c} | K22 | K23 | K24 |
| 010 | DI | Vg^{a} | Sw^{a} | BOW | NFLD | Jn^{a} | KREP | Tr^{a} | Fr^{a} | SW1 | | | |
| 021 | CROM | ZENA | CROV | CRAM | | | | | | | | | |

| | | Antigen number | | | | | | | | | | |
|---|---|---|---|---|---|---|---|---|---|---|---|---|
| System | | 025 | 026 | 027 | 028 | 029 | 030 | 031 | 032 | 033 | 034 | 035 |
| 002 | MNS | Dantu | Hop | Nob | En^{a} | En^{a}KT | ‘N’ | Or | DANE | TSEN | MINY | MUT |
| 004 | RH | . . . | c-like | cE | hr^{H} | Rh29 | Go^{a} | hr^{B} | Rh32 | Rh33 | Hr^{B} | Rh35 |
| 006 | KEL | VLAN | TOU | RAZ | VONG | KALT | KTIM | KYO | KUCI | KANT | KASH | |
| | | Antigen number | | | | | | | | | | |
| System | | 036 | 037 | 038 | 039 | 040 | 041 | 042 | 043 | 044 | 045 | 046 |
| 002 | MNS | SAT | ERIK | Os^{a} | ENEP | ENEH | HAG | ENAV | MARS | ENDA | ENEV | MNTD |
| 004 | RH | Be^{a} | Evans | . . . | Rh39 | Tar | Rh41 | Rh42 | Crawford | Nou | Riv | Sec |
| | | Antigen number | | | | | | | | | | |
| System | | 047 | 048 | 049 | 050 | 051 | 052 | 053 | 054 | 055 | 056 | 057 |
| 004 | RH | Dav | JAL | STEM | FPTT | MAR | BARC | JAHK | DAK | LOCR | CENR | CEST |

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of detecting an antibody of a G isotype (IgG antibody) against a red blood cell (RBC) antigen in a sample from a subject, comprising:
(a) contacting a first red blood cell membrane preparation (a rbcm preparation) comprising a first RBC antigen, with the sample from said subject, under conditions sufficient for the formation of an immune complex between said first RBC antigen and an anti-first-RBC antigen IgG antibody in said sample, wherein said first rbcm preparation is immobilized on a surface or substrate; and
(b) contacting a detection reagent with the immune complex of (a) under conditions sufficient for the formation of an immune complex between said detection reagent and the anti-first-RBC antigen IgG antibody in said sample, said detection reagent comprising an IgG-specific binding moiety,
thereby detecting an anti-RBC antigen IgG antibody in a sample.

2. The method of claim 1, wherein said IgG-specific binding moiety has one or more of the following properties:
(i) it comprises a heavy chain variable domain having a CDR comprising the amino acid sequence of ARSDGYYHYAMLDY (SEQ ID NO:38), or a CDR that has at least one amino acid alteration, but no more than two, three or four substitutions, deletions, or insertions, compared to compared to SEQ ID NO:38;
(ii) it comprises mAb MS-278, or an antigen binding fragment thereof;
(iii) it competes with mAb MS-278 for binding to IgG;
(iv) it comprises at least one antigen binding region from mAb MS-278;
(v) it comprises at least one, two or three complementarity determining regions (CDRs) from a heavy chain variable region of mAb MS-278;
vi) it comprises at least one, two or three CDRs from a light chain variable region of mAb MS-278;
(vii) it comprises a heavy chain variable region from mAb MS-278;
(viii) it comprises a light chain variable region from mAb MS-278;
(ix) it binds to an epitope bound by mAb MS-278;
(x) it binds to the rbcm preparation at a level, which is no more than 1.2, 1.5, 1.75, 2, 3, 4 or 5 times that of mAb MS-278;
(xi) it binds to IgG at a level which is at least 20, 30, 40, 50, 60, 70, 80, 90, or 100% of MS-278;
(xii) when bound to the rbcm preparation, at least 20, 40, 60% of said binding is to IgG;
(xiii) it binds to IgG with sufficient specificity that it can distinguish between the presence and absence of a pre-selected anti-red blood cell antigen in less than 30, 25, 20, 15, 10, or 5 minutes;
(xiv) it is substantially free of binding to the rbcm preparation;
(xv) its level of binding to a rbcm preparation is reduced by less than 10, 20, 30, 40, or 50% by pre-incubation of the rbcm preparation with an anti-IgG Fab or F(ab)₂ fragment;
(xvi) its level of binding to the rbcm preparation is reduced by less than 10, 20, 30, 40, or 50% by pre-incubation of the rbcm preparation with an enzyme that disrupts or alters an IgG- or an IgG-like molecule;
(xvii) its level of binding to the rbcm preparation is less than 1, 2, 5, 10, 25, or 50% of the binding of antibody chosen from 16H8 [Immucor], rabbit polyclonal [Alba #Z356], rabbit polyclonal [Biotest #804501], material from cell line CG-7 [Sigma-Aldrich I6260], or goat polyclonal [Sigma-Aldrich #I2136] to the rbcm preparation;
(xviii) it comprises an anti-IgG light chain antibody (mAb LCSIgG) chosen from Sigma-Aldrich #K4377 Cell Line KP-53, Sigma-Aldrich #L6522 cell line HP-6054, Sigma-Aldrich #K3502—polyclonal, or Sigma-Aldrich #L7646—polyclonal, or an antigen binding fragment thereof;
(xix) it competes with the mAb LCSIgG for binding to IgG;
(xx) it binds to an epitope bound by the mAb LCSIgG; or
(xxi) its level of binding to the rbcm preparation is less than 1, 2, 5, 10, 25, or 50% of the binding of mAb LCSIgG to the rbcm preparation.

3.-6. (canceled)

7. The method of claim 1, wherein said IgG-specific binding moiety comprises one or both of:
(i) a heavy chain variable domain that comprises three CDRs comprising the following sequences:
GFSLSTSGMGVS (SEQ ID NO:16) for CDR1, or a CDR that has at least one amino acid alteration, but no more than two, three or four substitutions, deletions, or insertions, compared to compared to SEQ ID NO:16;
HIYWDDDKRYNPSLKS (SEQ ID NO:22) for CDR2, or a CDR that has at least one amino acid alteration, but no more than two, three or four substitutions, deletions, or insertions, compared to compared to SEQ ID NO:22; and
ARSDGYYHYAMLDY (SEQ ID NO:38) for CDR3, or a CDR that has at least one amino acid alteration, but no more than two, three or four substitutions, deletions, or insertions, compared to compared to SEQ ID NO:38; or
(ii) a light chain variable domain that comprises three CDRs comprising the following sequences:
RASESVDSYGNSFMH (SEQ ID NO:2) for CDR1, or a CDR that has at least one amino acid alteration, but no more than two, three or four substitutions, deletions, or insertions, compared to compared to SEQ ID NO:2;
RASNLES (SEQ ID NO:3) for CDR2, or a CDR that has at least one amino acid alteration, but no more than two, three or four substitutions, deletions, or insertions, compared to compared to SEQ ID NO:3; and
QQTNEDPRT (SEQ ID NO:7) for CDR3, or a CDR that has at least one amino acid alteration, but no more than two, three or four substitutions, deletions, or insertions, compared to compared to SEQ ID NO:7.

8. The method of claim 1, wherein the IgG-specific binding moiety comprises one or both of:
(i) a heavy chain variable domain that comprises the amino acid sequence of SEQ ID NO:39, or an amino acid sequence at least 85%, 90%, 95%, or 99% identical to the amino acid sequence of SEQ ID NO: 39, or which differs by at least 1 or 5 residues, but less than 40, 30, 20, or 10 residues from SEQ ID NO: 39; or
(ii) a light chain variable domain that comprises the amino acid sequence of SEQ ID NO: 1, or an amino acid sequence at least 85%, 90%, 95%, or 99% identical to the amino acid sequence of SEQ ID NO: 1, or which differs by at least 1 or 5 residues, but less than 40, 30, 20, or 10 residues from SEQ ID NO: 1.

9.-10. (canceled)

11. The method claim 1, wherein said method comprises evaluating the sample from said subject for an antibody to at least 1, 2, 3, 4, 5, 6, 7, 8, 9, 10, 11, 12, or all of the following RBC antigens:
a Rhesus antigen chosen from one or more or all of D, C, c, E, or e;
a MNS antigen chosen from one or more or all of M, N, S, or s;
a Kidd antigen chosen from one or both of Jk^{a} or Jk^{b};
a Duffy antigen chosen from one or both of Fy^{a} or Fy^{b};
a Kell antigen chosen from one or both of K or k;
a Lewis antigen chosen from one or both of Le^{a} or Le^{b}; or
P antigen.

12. The method of claim 1, wherein the rbcm preparation provides a substrate having a density of between 14000-24000 cells/mm², 24000-34000 cells/mm², or 34000-40000 cells/mm², or 26,000 cells/mm² on the surface.

13. The method of claim 1, wherein said rbcm preparation is contacted with an agent that alters or disrupts an IgG or an IgG mimic before being contacted with the sample from said subject, thereby providing a mimic optimized rbcm preparation.

14. The method of claim 13, wherein the agent is an enzyme that cleaves the IgG or the IgG mimic.

15. The method of claim 1, where an angle between said surface or substrate, and the direction of an applied force that causes migration of the detection reagent, is non-orthogonal or other than 90 degrees,
wherein the applied force is chosen from one or more of a centrifugal, a gravitational, a fluid magnetic, an electric or a fluid force; and
wherein the centrifugal force is applied in at least two phases:
a first phase having FN1, the force normal to the surface or substrate, and FT1, the force tangential to said surface or substrate, and
a second phase having FN2, the force normal to the surface or substrate, and FT2, the force tangential to said surface or substrate,
wherein said first phase occurs before said second phase, wherein said angle is chosen as a constant angle during said first and second phase, and FN1 is greater than FN2, and FT1 is greater than FT2, or FN1 is less than FN2 and FT1 is less than FT2.

16.-18. (canceled)

19. The method of claim 1, wherein:
(i) the detection reagent is present at a concentration that results in coverage of less than or about 5%, 10%, 15%, 20%, 25% or 30% of the area of the surface or substrate;
(ii) the concentration of detection reagent is such that at least 30, 40, 50, 60, 70, 80, 90, or 100% of the surface or substrate is covered with at least a monolayer of the detection reagent;
(iii) the detection reagent is present in an amount that is at least 10, 20, 30, 40, 50, 60, 70, 80, 90, or 100 times the amount that would give 20% coverage of the substrate with a monolayer;
(iv) the positive readout is detected by having a uniform pattern of coverage of the surface or substrate by the detection reagent of at least 80%, 90%, 95%, 96%, 97%, 98%, 99% or 100% of the surface or substrate area; or
(v) a negative readout is detected by having a coverage of the surface or substrate by the detection reagent of less than 99%, 95%, 90%, 85%, 80%, 75%, 70%, 60%, 50%, 40% or 30% of the surface or substrate area relative to what would be covered in a positive sample.

20.-23. (canceled)

24. A method of evaluating a sample for a red blood cell (RBC) antigen-specific antibody for reverse grouping or typing, comprising:
(a) contacting a rbcm preparation which specifically presents or lacks one or more red blood cell antigens disposed as a substrate or a surface, with a sample, under conditions sufficient for the formation of a complex between said rbcm preparation and an anti-red blood cell antigen-specific antibody, in said sample;
(b) contacting one or more indicator cells which specifically present or lack said red blood cell antigen with the complex of (a), under conditions sufficient for the formation of an immune complex between said rbcm preparation and the indicator cells;
(c) providing a multi-valent binding agent that can promote clumping between the indicator cells, under conditions sufficient for the formation of the immune complex, of said indicator cells, via said multi-valent binding agent,
(d) applying an acceleration force chosen from a centrifugal, a gravitational, a fluid magnetic, an electric or a fluid, force,
wherein said indicator cells indicate the presence or absence of said red blood cell antigen by the distribution of indicator cells, or by the strength of adhesion of unbound indicator cells to the substrate or surface,
thereby evaluating said sample.

25. The method of claim 24, wherein the indicator cell is a red blood cell chosen from one or more of A+, B+, or O+ indicator cells.

26.-30. (canceled)

31. The method of claim 24, wherein the multi-valent binding agent comprises one or both of:
(i) a heavy chain variable domain that comprises three CDRs comprising the following sequences:
GFSLSTSGMGVS (SEQ ID NO:16) for CDR1, or a CDR that has at least one amino acid alteration, but no more than two, three or four substitutions, deletions, or insertions, compared to compared to SEQ ID NO:16;
HIYWDDDKRYNPSLKS (SEQ ID NO:22) for CDR2, or a CDR that has at least one amino acid alteration, but no more than two, three or four substitutions, deletions, or insertions, compared to compared to SEQ ID NO:22; and
ARSDGYYHYAMLDY (SEQ ID NO:38) for CDR3, or a CDR that has at least one amino acid alteration, but no more than two, three or four substitutions, deletions, or insertions, compared to compared to SEQ ID NO:38; or
(ii) a light chain variable domain that comprises three CDRs comprising the following sequences:
RASESVDSYGNSFMH (SEQ ID NO:2) for CDR1, or a CDR that has at least one amino acid alteration, but no more than two, three or four substitutions, deletions, or insertions, compared to compared to SEQ ID NO:2;
RASNLES (SEQ ID NO:3) for CDR2, or a CDR that has at least one amino acid alteration, but no more than two, three or four substitutions, deletions, or insertions, compared to compared to SEQ ID NO:3; and
QQTNEDPRT (SEQ ID NO:7) for CDR3, or a CDR that has at least one amino acid alteration, but no more than two, three or four substitutions, deletions, or insertions, compared to compared to SEQ ID NO:7.

32. The method of claim 24, wherein the multi-valent binding agent comprises one or both of:
(i) a heavy chain variable domain that comprises the amino acid sequence of SEQ ID NO:39, or an amino acid sequence at least 85%, 90%, 95%, or 99% identical to the amino acid sequence of SEQ ID NO:39, or which differs by at least 1 or 5 residues, but less than 40, 30, 20, or 10 residues from SEQ ID NO:39; or
(ii) a light chain variable domain that comprises the amino acid sequence of SEQ ID NO: 1, or an amino acid sequence at least 85%, 90%, 95%, or 99% identical to the amino acid sequence of SEQ ID NO: 1, or which differs by at least 1 or 5 residues, but less than 40, 30, 20, or 10 residues from SEQ ID NO: 1.

33. (canceled)

34. The method of claim 24, wherein the multivalent binding agent is an anti-D antibody; the rbcm preparation is negative for D antigen; and the indicator cells are positive for D antigen.

35. The method of claim 24, wherein said rcbm is immobilized on a surface or substrate, and the angle between said surface or substrate, and the direction of an applied force, that causes migration of said indicator cells, is non-orthogonal or other than 90 degrees, wherein the centrifugal force is applied in two phases:
a first phase having FN1, the force normal to the surface, and FT1, the force tangential to said surface or substrate, and
a second phase having FN2, the force normal to the surface, and FT2, the force tangential to said surface or substrate,
wherein said first phase occurs before said second phase, and one or both of the following is true: FN1 is greater than FN2, and FT2 is greater than FT1.

36. (canceled)

37. A method of detecting an analyte in a sample, comprising:
(a) contacting a capture agent with the sample, under conditions sufficient for the formation of a complex between the capture agent and said analyte in said sample,
wherein, said capture agent is immobilized on a substrate or a surface, and the angle between said substrate or a surface and the direction of an applied force chosen from a centrifugal, a gravitational, a fluid magnetic, an electric or a fluid, force, that causes migration of detection reagent, is non-orthogonal or other than 90 degrees;
(b) contacting a detection reagent with the complex of (a) under conditions sufficient for the formation of a complex between said detection reagent and the analyte in said sample,
(c) applying a centrifugal acceleration force at an angle such that the detection reagent that does not bind to said capture agent migrates across said substrate or surface,
thereby detecting an analyte in a sample.

38. The method of claim 37, wherein:
(i) the capture agent is an antibody, an anti-RBC antibody, an antigen, an RBC antigen, an rbcm preparation, or an optimized rbcm preparation;
(ii) the analyte is chosen from an antigen, an antibody or other protein having specific binding for said capture agent; or
(iii) said detection reagent can comprise a red blood cell and one or more immunoglobulin binding agents as an indicator moiety.

39.-44. (canceled)

45. The method of claim 37, wherein said detection reagent comprises one or both of:
(i) a heavy chain variable domain that comprises three CDRs comprising the following sequences:
GFSLSTSGMGVS (SEQ ID NO:16) for CDR1, or a CDR that has at least one amino acid alteration, but no more than two, three or four substitutions, deletions, or insertions, compared to compared to SEQ ID NO:16;
HIYWDDDKRYNPSLKS (SEQ ID NO:22) for CDR2, or a CDR that has at least one amino acid alteration, but no more than two, three or four substitutions, deletions, or insertions, compared to compared to SEQ ID NO:22; and
ARSDGYYHYAMLDY (SEQ ID NO:38) for CDR3, or a CDR that has at least one amino acid alteration, but no more than two, three or four substitutions, deletions, or insertions, compared to compared to SEQ ID NO:38; or
(ii) a light chain variable domain that comprises one, two, or three CDRs comprising the following sequences:
RASESVDSYGNSFMH (SEQ ID NO:2) for CDR1, or a CDR that has at least one amino acid alteration, but no more than two, three or four substitutions, deletions, or insertions, compared to compared to SEQ ID NO:2;
RASNLES (SEQ ID NO:3) for CDR2, or a CDR that has at least one amino acid alteration, but no more than two, three or four substitutions, deletions, or insertions, compared to compared to SEQ ID NO:3; and
QQTNEDPRT (SEQ ID NO:7) for CDR3, or a CDR that has at least one amino acid alteration, but no more than two, three or four substitutions, deletions, or insertions, compared to compared to SEQ ID NO:7.

46. The method of claim 40, wherein said detection reagent comprises one or both of:
(i) a heavy chain variable domain that comprises the amino acid sequence of SEQ ID NO:39, or an amino acid sequence at least 85%, 90%, 95%, or 99% identical to the amino acid sequence of SEQ ID NO: 39, or which differs by at least 1 or 5 residues, but less than 40, 30, 20, or 10 residues from SEQ ID NO: 39; or
(ii) a light chain variable domain that comprises the amino acid sequence of SEQ ID NO: 1, or an amino acid sequence at least 85%, 90%, 95%, or 99% identical to the amino acid sequence of SEQ ID NO: 1, or which differs by at least 1 or 5 residues, but less than 40, 30, 20, or 10 residues from SEQ ID NO: 1.

47.-48. (canceled)

49. The method of claim 37, wherein the method is applied to one or more forward typing or grouping, reverse typing or grouping, antibody screening, antibody identification, extended phenotyping, or pathogen analysis, alone or in combination.

50. The method of claim 37, wherein the capture agent comprises:
(i) at least 1, 2, 3, 4, 5, 6, 9, 10, 11, 12 or all of an RBC antigens chosen from: a Rhesus antigen chosen from one or more or all of D, C, c, E, or e; an MNS antigen chosen from one or more or all of M, N, S, or s; a Kidd antigen chosen from one or both of Jk^{a} or Jk^{b}; a Duffy antigen chosen from one or both of Fy^{a} or Fy^{b}; a Kell antigen chosen from one or both of K or k; a Lewis antigen chosen from one or both of Le^{a} or Le^{b}; or P antigen; or
(ii) an antibody against one or more of: a Rhesus antigen chosen from one or more or all of D, C, c, E, or e; an MNS antigen chosen from one or more or all of M, N, S, or s; a Kidd antigen chosen from one or both of Jk^{a} or Jk^{b}; a Duffy antigen chosen from one or both of Fy^{a} or Fy^{b}; a Kell antigen chosen from one or both of K or k; a Lewis antigen chosen from one or both of Le^{a} or Le^{b}; or a P antigen.

51.-63. (canceled)

64. A kit comprising a detection reagent having an indicator moiety and a binding moiety, wherein said kit comprises one or more, or all of:
(a) a rbcm preparation or a mimic optimized-rbcm preparation;
(b) a detection reagent complexing agent that promotes detection reagent complexation between base units of detection reagent;
(c) a positive control sample, said positive control sample having an antibody to a preselected blood type antigen;
(d) a negative control sample, said negative control sample lacking an antibody to a preselected blood type antigen; and
(e) an agent that alters or disrupts an IgG molecule or an IgG-like molecule for preparing a mimic optimized-rbcm preparation.

65. (canceled)


SEQUENCE LISTING (45 sequences)

SEQ ID NO: 1 - protein, 213 aa, Artificial Sequence
  source: /note=“Description of Artificial Sequence Synthetic polypeptide”
DIVLTQSPASLAVSLGQRATISCRASESVDSYGNSFMHWYQQKPGQPPKLLIYRASNLESGIPARFSGSGSGTDFTLTINPVEADDVATYYCQQTNEDPRTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSF

SEQ ID NO: 2 - protein, 15 aa, Artificial Sequence
  source: /note=“Description of Artificial Sequence Synthetic peptide”
RASESVDSYGNSFMH

SEQ ID NO: 3 - protein, 7 aa, Artificial Sequence
  source: /note=“Description of Artificial Sequence Synthetic peptide”
RASNLES

SEQ ID NO: 4 - protein, 4 aa, Artificial Sequence
  source: /note=“Description of Artificial Sequence Synthetic peptide”
DPRT

SEQ ID NO: 5 - protein, 10 aa, Artificial Sequence
  source: /note=“Description of Artificial Sequence Synthetic peptide”
QAGTNEDPRT

SEQ ID NO: 6 - protein, 10 aa, Artificial Sequence
  source: /note=“Description of Artificial Sequence Synthetic peptide”
QAGTNEDPRT

SEQ ID NO: 7 - protein, 9 aa, Artificial Sequence
  source: /note=“Description of Artificial Sequence Synthetic peptide”
QQTNEDPRT

SEQ ID NO: 8 - protein, 10 aa, Artificial Sequence
  source: /note=“Description of Artificial Sequence Synthetic peptide”
AGQTENDPRT

SEQ ID NO: 9 - protein, 10 aa, Artificial Sequence
  source: /note=“Description of Artificial Sequence Synthetic peptide”
AGQTNEDPRT

SEQ ID NO: 10 - protein, 10 aa, Artificial Sequence
  source: /note=“Description of Artificial Sequence Synthetic peptide”
FPLGVSDPRT

SEQ ID NO: 11 - protein, 10 aa, Artificial Sequence
  source: /note=“Description of Artificial Sequence Synthetic peptide”
GAQTENDPRT

SEQ ID NO: 12 - protein, 10 aa, Artificial Sequence
  source: /note=“Description of Artificial Sequence Synthetic peptide”
QGATNEDPRT

SEQ ID NO: 13 - protein, 10 aa, Artificial Sequence
  source: /note=“Description of Artificial Sequence Synthetic peptide”
QQTGGEDPRT

SEQ ID NO: 14 - protein, 243 aa, Artificial Sequence
  source: /note=“Description of Artificial Sequence Synthetic polypeptide”
QVTLKESGPGILQPSQTLSLTCSFSGFSLSTSGMGVSWIRQPSGKGLEWLAHIYWDDDKRYNPSLKSRLTISKDTSRNQVFLKITSVDTADTATYYCARXXXXXXXLDYWGQGTTLTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDG

SEQ ID NO: 15 - protein, 13 aa, Artificial Sequence
  source: /note=“Description of Artificial Sequence Synthetic peptide”
GFASLSTSGMGVS

SEQ ID NO: 16 - protein, 12 aa, Artificial Sequence
  source: /note=“Description of Artificial Sequence Synthetic peptide”
GFSLSTSGMGVS

SEQ ID NO: 17 - protein, 13 aa, Artificial Sequence
  source: /note=“Description of Artificial Sequence Synthetic peptide”
GYASLSTSGMGVS

SEQ ID NO: 18 - protein, 13 aa, Artificial Sequence
  source: /note=“Description of Artificial Sequence Synthetic peptide”
YGASLSTSGMGVS

SEQ ID NO: 19 - protein, 6 aa, Artificial Sequence
  source: /note=“Description of Artificial Sequence Synthetic peptide”
MEEFLL

SEQ ID NO: 20 - protein, 6 aa, Artificial Sequence
  source: /note=“Description of Artificial Sequence Synthetic peptide”
LLLFGL

SEQ ID NO: 21 - protein, 6 aa, Artificial Sequence
  source: /note=“Description of Artificial Sequence Synthetic peptide”
NSDYYK

SEQ ID NO: 22 - protein, 16 aa, Artificial Sequence
  source: /note=“Description of Artificial Sequence Synthetic peptide”
HIYWDDDKRYNPSLKS

SEQ ID NO: 23 - protein, 13 aa, Artificial Sequence
  source: /note=“Description of Artificial Sequence Synthetic peptide”
DYVQEDISKDTSR

SEQ ID NO: 24 - protein, 13 aa, Artificial Sequence
  source: /note=“Description of Artificial Sequence Synthetic peptide”
FMVQEDISKDTSR

SEQ ID NO: 25 - protein, 13 aa, Artificial Sequence
  source: /note=“Description of Artificial Sequence Synthetic peptide”
DYNLEDISKDTSR

SEQ ID NO: 26 - protein, 9 aa, Artificial Sequence
  source: /note=“Description of Artificial Sequence Synthetic peptide”
LFFVPHISK

SEQ ID NO: 27 - protein, 9 aa, Artificial Sequence
  source: /note=“Description of Artificial Sequence Synthetic peptide”
RHNVPHISK

SEQ ID NO: 28 - protein, 9 aa, Artificial Sequence
  source: /note=“Description of Artificial Sequence Synthetic peptide”
AALQELISK

SEQ ID NO: 29 - protein, 11 aa, Artificial Sequence
  source: /note=“Description of Artificial Sequence Synthetic peptide”
FKTVDRTISKD

SEQ ID NO: 30 - protein, 11 aa, Artificial Sequence
  source: /note=“Description of Artificial Sequence Synthetic peptide”
GYRVDRTISKD

SEQ ID NO: 31 - protein, 10 aa, Artificial Sequence
  source: /note=“Description of Artificial Sequence Synthetic peptide”
VEAFQTTISK

SEQ ID NO: 32 - protein, 10 aa, Artificial Sequence
  source: /note=“Description of Artificial Sequence Synthetic peptide”
NNAFKTTISK

SEQ ID NO: 33 - protein, 10 aa, Artificial Sequence
  source: /note=“Description of Artificial Sequence Synthetic peptide”
DIAFQTTISK

SEQ ID NO: 34 - protein, 10 aa, Artificial Sequence
  source: /note=“Description of Artificial Sequence Synthetic peptide”
YPEAWETISK

SEQ ID NO: 35 - protein, 10 aa, Artificial Sequence
  source: /note=“Description of Artificial Sequence Synthetic peptide”
TYYCARTTGY

SEQ ID NO: 36 - protein, 10 aa, Artificial Sequence
  source: /note=“Description of Artificial Sequence Synthetic peptide”
TYYCARSDGY

SEQ ID NO: 37 - protein, 13 aa, Artificial Sequence
  source: /note=“Description of Artificial Sequence Synthetic peptide”
DYWGQGTSVTVSS

SEQ ID NO: 38 - protein, 14 aa, Artificial Sequence
  source: /note=“Description of Artificial Sequence Synthetic peptide”
ARSDGYYHYAMLDY

SEQ ID NO: 39 - protein, 245 aa, Artificial Sequence
  source: /note=“Description of Artificial Sequence Synthetic polypeptide”
QVTLKESGPGILQPSQTLSLTCSFSGFSLSTSGMGVSWIRQPSGKGLEWLAHIYWDDDKRYNPSLKSRLTISKDTSRNQVFLKITSVDTADTATYYCARSDGYYHYAMLDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDG

SEQ ID NO: 40 - protein, 4 aa, Artificial Sequence
  source: /note=“Description of Artificial Sequence Synthetic peptide”
ANNA

SEQ ID NO: 41 - protein, 4 aa, Artificial Sequence
  source: /note=“Description of Artificial Sequence Synthetic peptide”
NANA

SEQ ID NO: 42 - protein, 4 aa, Artificial Sequence
  source: /note=“Description of Artificial Sequence Synthetic peptide”
ANAN

SEQ ID NO: 43 - protein, 4 aa, Artificial Sequence
  source: /note=“Description of Artificial Sequence Synthetic peptide”
NAAN

SEQ ID NO: 44 - protein, 4 aa, Artificial Sequence
  source: /note=“Description of Artificial Sequence Synthetic peptide”
NNAA

SEQ ID NO: 45 - protein, 4 aa, Artificial Sequence
  source: /note=“Description of Artificial Sequence Synthetic peptide”
AANN